(19) United States
(12) Patent Application Publication
(10) Pub. No.: US 2006/0052582 A1
(43) Pub. Date: Mar. 9, 2006

(54) Identification of inhibitors of mitosis

(76) Inventors: Joan Roig Amores, Boston, MA (US); Christopher Belham, Parabiago (IT); Joseph Avruch, Brookline, MA (US)

Correspondence Address: Leon R Yankwich;Yankwich & Associates, 201 Broadway, Cambridge, MA, 02139, US

(21) Appl. No.: 10/517,622

(22) PCT Filed: Jun. 11, 2003
(86) PCT No.: PCT/US03/19743

§ 371 Date: Dec. 10, 2004

Related U.S. Application Data
(60) Provisional application No. 60/387,810, filed on Jun. 11, 2002.

(51) Int. Cl. C12Q 1/48

(52) U.S. Cl. 530/344; 435/15

(57) ABSTRACT

Methods and compositions are described for using NIMA-like kinases to identify anti-mitotic compounds and to diagnose cancer.

### STATEMENT REGARDING U.S. SPONSORED RESEARCH

This invention described herein was supported in whole or in part by National Institutes of Health grant DK17776. The United States Government has certain rights in the invention.

### FIELD OF THE INVENTION

This invention is generally in the field of identifying anti-cancer and antimicrobial compounds. More specifically, the invention provides methods of screening for and identifying compounds that may be used to treat cancer based on the ability of the compounds to inhibit cell mitosis and proliferation by inhibiting the activity of one or more NIMA family kinases involved in mitosis in eukaryotic cells.

### BACKGROUND

The progression of a proliferating eukaryotic cell through phases of the cell cycle is controlled by an array of regulatory proteins, which guarantee that mitosis occurs at the appropriate time. The eukaryotic cell cycle includes a growth phase and a reproductive phase; the latter composed of the chromosome cycle and the centrosome cycle, which intersect in the establishment of the mitotic apparatus. The process of mitosis, whereby a single cell divides with fidelity into two sister cells, is normally tightly regulated such that even during periods of rapid division as in the developing fetus, growth spurts in vertebrates, and wound repair, control is maintained to prevent unchecked proliferation of individual lines of undifferentiated cells or particular cell and tissue types, i.e., to prevent the development of cancer.

Thus, cell division is normally timed and controlled. Such control is accomplished in part through the interplay of a specialized set of mutually antagonistic kinases and phosphatases, the best known of which are the cyclin-dependent kinases (CDKs). Protein kinases can be divided into two main groups based on either amino acid sequence similarity or specificity for either serine/threonine or tyrosine residues. The protein kinases of most kinase families also share structural features outside the kinase domain that reflect their particular cellular roles. Recent studies indicate that several other families of protein kinases also play important roles at different stages of the intricate process of making two cells out of one (Nigg et al., Nature Reviews, 2: 21-32 (2001). For example, Polo-like kinases (Glover et al., Genes Dev., 12: 3777-37887 (1998), Aurora kinases, and NIMA-like kinases (Neks, Fry et al., Methods Enzymol., 283: 270-282 (1997); Kandli et al., Genomics, 68: 187-196 (2000)) have been implicated in such steps as centrosome separation and chromosome condensation in prophase, nuclear envelope breakdown and spindle assembly in prometaphase, as well as in exit from mitosis and cytokinesis.

The term “NIMA” is derived from the Aspergillus nidulans protein kinase encoded by the nimA gene. Early data from Aspergillus suggested that NIMA cooperates with p34^{Cdc2}/cyclin B during the onset of mitosis. More recent evidence has indicated that NIMA is necessary for nuclear entry of p34^{Cdc2}/cyclin B (Wu et al., J. Cell Biol., 141: 1575-1587 (1998)). Moreover, both Cdc2 and NIMA must be inactivated for mitotic exit. Temperature-sensitive mutations of the NIMA gene (Osmani et al., Cell, 67: 283-291 (1991)), or expression of the noncatalytic domain of NIMA kinase (Lu et al., EMBO J., 13: 2103-2113 (1994)), can arrest Aspergillus cells in the G2 cell cycle (thus, the observation of “never in mitosis”, abbreviated “NIMA” to classify such kinases), without interfering with p34^{Cdc2} activation. Conversely, over-expression of a NIMA causes chromatin condensation and abnormal spindle formation without activating p34^{Cdc2} (O'Connell et al., EMBO J, 13: 4926-4937 (1994); Osmani et al., Cell, 52: 241-251 (1988)). G2 arrest of NIMA mutants can be bypassed by mutation of different anaphase-promoting complex (APC) subunits. Double NIMA and APC temperature-sensitive mutants can enter mitosis when shifted to restrictive temperature, although mitotic cells show aberrant nuclear envelope and spindle organization, pointing to an involvement of the NIMA protein kinase in mitotic processes beyond the control of the G2/M transition in the cell cycle (Osamani et al., Cell, 52: 241-251 (1988); Osamani et al., EMBO J., 10: 2669-2679 (1991); Lies et al., J. Cell Sci., 111: 1453-1465 (1998)).

NIMA-related (or NIMA-like) kinases are also referred to as “Neks”. As with the original NIMA, Neks are serine/threonine (“ser/thr”) kinases that are biochemically distinct from other protein kinases and have a phosphotransferase activity, which is regulated by ser/thr phosphorylation. A NIMA or NIMA-like kinase-mediated mitotic pathway appears to be present in most eukaryotic cells, ranging from fungi, such as Aspergillus (the first reported source of a NIMA kinase), to vertebrate cells (Lu and Hunter, Cell, 81: 413, 1995a). In Xenopus oocytes, NIMA induces germinal vesicle breakdown without activating Mos, Cdc2 or MAP kinase. In HeLa cells, NIMA induces mitotic events without activating Cdc2, whereas dominant-negative NIMA mutants cause a specific arrest at the G2 cell cycle. In addition, O'Connell et al. (EMBO J. 13: 4926-4937 (1994)) have demonstrated that NIMA induces premature chromatin condensation in fission yeast and HeLa cells. Clearly, NIMA and NIMA-like kinases play a critical role in the orderly regulation of mitosis of eukaryotic cells.

NIMA protein levels change greatly during the cell cycle, being maximal during mitosis, and NIMA protein kinase activity seems to parallel NIMA protein content. NIMA is hyper-phosphorylated in vivo during mitosis and can be phosphorylated in vitro by p34^{Cdc2} (Ye et al., EMBO J., 14: 986-994 (1995)). Such in vitro phosphorylation alters NIMA protein kinase activity modestly. However, once phosphorylated in mitosis, NIMA is rapidly degraded, and this degradation is necessary for mitotic exit (see, e.g., O'Connell et al., EMBO J., 11: 2139-2149 (1992); Pe et al., EMBO J., 14: 995-1003 (1995)).

The ability of recombinant NIMA to induce chromatin condensation in fission yeast (O'Connell et al., EMBO J., 13: 4926-4937 (1994)) and vertebrate cells (accompanied in the latter by nuclear membrane breakdown; see, e.g, O'Connell et al., EMBO J., 13: 4926-4937 (1994); Lu et al., Cell, 81: 413-424 (1995)) as occurs in Aspergillus, supports the general view that a NIMA or NIMA-like protein kinase with similar specificity participates in cell cycle control in all metazoans. Protein kinases structurally related to NIMA have been isolated from several phyla, including at least seven mammalian “Neks” (see, e.g., Nigg, Nature Reviews, 2: 21-32 (2001)). However, none of these Neks have emerged as a bona fide functional homologue of NIMA, i.e., as necessary for mitotic progression, or able to induce chromatin condensation if overexpressed. Neks are most closely related to NIMA in their catalytic domain sequences but diverge substantially from NIMA in their noncatalytic carboxy terminal tails. This is also true for the Neurospora crassa NIMA-related kinase that has the capacity to complement the NIMA mutation (Pu et al., J. Biol. Chem., 270: 18110-18116 (1995)). Nek2, the mammalian homologue most similar in overall amino acid sequence to NIMA, is involved in the regulation of centrosomal structure and function (Mayor et al., FEBS Lett., 452: 92-95 (1999)), but does not appear to be involved in other aspects of mitosis. The functions of other Neks are largely unknown, although Nek6, and the closely related Nek7, recent mammalian additions to the family (Kandli et al., Genomics, 68: 187-196 (2000)), have been shown to phosphorylate the protein kinase p70 S6 kinase on Thr412 within a hydrophobic motif, a phosphorylation which, together with the PDK1-catalyzed phosphorylation of Thr252 in the activation loop, mediates activation of the p70 S6 kinase (Belham et al., Curr. Biol., 11: 1155-1167 (2001)).

The above discussion clearly indicates that NIMA or NIMA-like kinases may play critical roles in progression into and exiting mitosis. Accordingly, such kinases provide attractive molecular targets for developing anti-proliferative agents to treat cancer, microbial infections, and other conditions characterized by an unchecked or aberrant mitotic process.

### SUMMARY OF THE INVENTION

The invention provides methods of screening compounds for the ability to inhibit or interrupt mitosis based on the ability to specifically inhibit the activity of one or more NIMA-like kinases, particularly, Nercc1 kinase, Nek6 kinase, and Nek7 kinase (a homolog of Nek6), which are involvde in a cascade of kinases that regulates mitotic progression of eukaryotic cells. Nercc1 kinase is a NIMA-like protein kinase (also referred herein as “Nercc kinase”,“Nercc1”, “Nerrc”, “Nek9”) that activates Nek6 and/or its homolog, Nek7, by phosphorylating Nek6 and/or Nek7, at one or more specific phosphorylation sites on the proteins in a step that is critical for a cell to enter and maintain mitosis. Nercc1 kinase is also able to auto-activate by auto-phosphorylation. Activated (i.e., phosphorylated) forms of Nek6 and Nek7, in turn, phosphorylate another target in the cascade to signal mitotic progression of the eukaryotic cell. Thus, compounds that inhibit Nercc1, Nek6, or Nek7 activity will inhibit a critical step in regulating and maintaining mitosis. Such compounds are candidates for use in treating conditions of uncontrolled mitotic progression, such as in cancer and/or eukaryotic microbial infections (e.g., by fungi, parasitic protozoa, parasitic helminths).

The invention provides compositions and methods that may be used to determine whether a test compound is an inhibitor of mitosis (i.e., an anti-mitotic compound) based on the ability to inhibit Nercc1 kinase, Nek6, or Nek7, which play critical roles in a cell's entry into and maintenance of mitotic progression.

In one embodiment, the invention provides a method of identifying a compound that is an inhibitor of mitosis comprising:
(a) providing a kinase reaction mixture comprising a purine nucleoside triphosphate, a Nercc1 kinase protein, and a kinase substrate,
(b) incubating said kinase reaction mixture in the presence and absence of a test compound for a time sufficient to permit the Nercc1 kinase protein to phosphorylate said kinase substrate, and
(c) detecting the level of phosphorylated kinase substrate produced in the presence and absence of said test compound,
   wherein a lower level of phosphorylated kinase substrate produced in the presence of said test compound compared to the level produced in the absence of said test compound indicates that said test compound is an inhibitor of mitosis.

In another embodiment, the invention provides a method of identifying a test compound that is an inhibitor of mitosis comprising:
(a) providing a kinase reaction mixture comprising an activated Nek6 or Nek7 kinase protein, a linase substrate, and a purine nucleoside triphosphate,
(b) incubating said reaction mixture in the presence and absence of a test compound for a time sufficient to permit the activated Nek6 or Nek7 linase protein to phosphorylate said kinase substrate, and
(c) detecting the level of phosphorylated kinase substrate in the presence and absence of said test,
   wherein a lower level of phosphorylated kinase substrate produced in the presence of said test compound compared to the level produced in the absence of said test compound indicates that said test compound is an inhibitor of mitosis.

A variety of Nercc1 kinase, Nek6, and Nek7 kinase proteins may be used in the invention. Such kinase proteins may be isolated from a biological source, produced by recombinant methods, or produced by a combination of recombinant and synthetic methods. Kinase proteins useful in the invention also include permanently activated, mutant variant of the wild type kinase. For example, a particularly preferred mutant variant of the Nercc1 kinase useful in the invention lacks the entire RCC1 auto-inhibitory domain of Nercc, such as the Nercc (Δ347-732) linase protein. Such RCC1-deleted Nercc variant proteins are permanently (constitutively) activated and, therefore, provide a fully functional Nercc1 kinase activity without the necessity of undergoing phosphorylation for activation as in wild type Nercc1 kinase. Protein kinase proteins useful in the invention also include various fusion proteins comprising all or a portion of a Nercc1, Nek6, and/or Nek7 protein. Particularly preferred are fusion proteins comprising a Nercc1, Nek6, or Nek7 kinase polypeptides and any of a variety of epitope peptides (epitope tags) including, but not limited to, FLAG, HA (hemaglutinin tag), myc (c-myc tag), and combinations thereof, which provide a fusion protein with an amino acid sequence (tag) that is readily detected or labeled by standard methods and compositions, e.g., employing readily available tag-specific antibodies or affinity resins. A particularly preferred self-activating fusion protein useful in the invention comprises a Nercc1 or catalytically active portion thereof fused to glutathione-S-transferase (GST). GST fusion proteins are conveniently isolated from a mixture by affinity chromatography using a glutatione adduct resin.

Kinase substrates for Nercc1, Nek6, or Nek7 kinase-mediated phosphorylation useful in the compositions and methods of the invention may be any polypeptide that comprises a domain that is susceptible to phosphorylation by the particular kinase protein employed. As Nercc1 linase is able to auto-phosphorylate, a Nercc1 kinase protein used in a method of the invention may be initially provided as activated (phosphorylated) or non-activated (non-phosphorylated) in a method described herein. The ability to auto-phosphorylate and auto-activate also enables the use of a non-activated Nercc1 kinase protein to be employed as both the Nercc1 kinase protein as well as the kinase substrate in methods of the invention. Preferred kinase substrates useful in the invention include, without limitation, a non-activated Nercc1 kinase protein, a non-activated Nek6, a non-activated Nek7, a histone (e.g., histone H3, histone H4), casein, myelin basic protein (MBP), Cdc16, or a fusion protein comprising any of these proteins. Cdc16 is particularly preferred as a kinase substrate for Nek6 and Nek7. Fusion proteins useful as kinase substrates in the methods of the invention preferably are readily isolated, labeled, and/or detected in phosphorylated form. For example, a recombinant fusion protein comprising glutathione S-transferase (GST) and Nek6 (GST Nek6) or Nek7 (GST Nek7) is a preferred substrate for Nercc1 kinase-mediated phosphorylation.

The invention also provides reaction mixtures that may be used to test a compound for the ability to inhibit mitosis based on the ability of the compound inhibit a kinase activity of a NIMA-like protein kinase, as described herein. A preferred reaction mixture of the invention comprises a purine nucleoside triphosphate, a NIMA-like protein kinase described herein, and a linase substrate. A NIMA-like protein kinase useful in reaction mixtures of the invention is activated or is able to be activated to provide kinase activity that phosphorylates a kinase substrate. NIMA-like protein kinases useful in a reaction mixture of the invention include, but are not limited to, Nercc1 kinase; Nek6 kinase; Nek7 kinase; fusion proteins comprising Nercc1, Nek6, or Nek7; mutant variants comprising Nercc1, Nek6, or Nek7; and combinations thereof, provided that such NRAA-like protein kinase is activated or can be activated to provide kinase activity that phosphorylates a kinse substrate of the reaction mixture. A preferred kinase substrate useful in reaction mixtures of the invention is a non-phosphorylated (non-activated) NIMA-like protein kinase (e.g., a non-activated Nercc1, Nek6, or Nek7 protein), a histone (e.g., histone H3, histone H4), a casein, a myelin binding protein (MBP), and combinations thereof. As wild type Nercc1 can auto-activate by auto-phosphorylation, a non-activated Nercc1 may be employed as both the NIMA-like protein kinase and the kinase substrate in a reaction mixture of the invention.

Purine nucleoside triphosphate useful in the compositions and methods of the invention is preferably adenosine triphosphate (ATP) or guanosine triphosphate (GTP). Using GTP provides an added specificity to the Nercc1 kinase reaction because most other kinases that could interfere in various biological samples are unable to utilize GTP to phosphorylate a protein substrate. A metal cation (counter ion), such as a divalent magnesium or a divalent manganese cation, is required for all in vitro phosphorylation reactions of the invention. Divalent magnesium cations are a particularly preferred metal counter ion for methods and compositions described herein.

A phosphorylated kinase substrate (i.e., a phosphorylated kinase reaction product) of Nercc1 or Nek6M kinase activities may be detected using any of a variety of means available for detecting phosphorylated proteins. If Nercc, Nek6, or Nek7 are used as a kinase substrate, phosphorylated forms of these proteins may be determined by increased kinase activity in a standard enzyme reaction. A preferred detection means is the use of an antibody that specifically binds the phosphorylated form of a particular linase substrate, such as an antibody specific for Nek6 or portion thereof that is phosphorylated at a particular phosphorylation sites involved in kinase activation, e.g., serine 206 (Ser206) in the activation loop of the Nek6 protein or Ser195 in the activation loop of the Nek7 protein. Antibodies that specifically bind a phosphorylated kinase substrate permit the use of any of the various immunodetection systems available in the art. Alternatively, a phosphorylated kinase substrate may be detected directly by employing a purine nucleoside triphosphate that is labeled in the phosphate group with a detectable label or with a component of a phosphorylated product detection system, such as a radiolabel phosphorous isotope (³²P), or a non-radioactive, detectable group that is transferred to the kinase substrate during phosphorylation.

A compound identified as an inhibitor of mitosis (i.e., “anti-mitotic compound”) based on inhibition of a NIMA-like protein kinase activity in a reaction mixture of the invention may be further tested for the ability to halt mitosis in cells, either in vitro as in cells in cultures undergoing mitosis or in vivo in animal models, such as animal models for tumors and other cancers. Particularly preferred is a further step in which dividing (mitotic) cells are contacted with the compound and assayed for some affect on cellular DNA and/or microtubules. For example, such further testing may include determining whether the desired compound causes a disruption of the spindles (mitotic microtubules), disruption of the alignment of chromosomes as revealed by standard staining methods, or promotion of apoptosis.

One or more steps of a method of the invention may be carried out in any of a variety of formats used to assay kinase reactions, including individual test tubes, wells of microtiter plates, and on biochips, which permit hundreds or even thousands of compounds to be tested for the ability to inhibit a Nercc and/or Nek6/7 kinase reaction(s) simultaneously.

In another embodiment, the invention provides a method of diagnosing a cancerous or potentially cancerous condition in cells based on detecting an unusual elevation in the level of Nercc1 kinase, Nek6, and/or Nek7 protein expression or in the level of the corresponding kinase activity. Cells for such an analysis may be obtained from any of a variety of sources, including but not limited to, tissue biopsies, blood, smears, tissue swabs, and other body fluid samples.

### BRIEF DESCRIPTION OF THE DRAWINGS

FIG. 1 is a diagram of various domains in the full-length (979 amino acids) Nercc1 kinase protein. Abbreviations: “Protein Kinase”, Nercc1 kinase catalytic domain; “NLS”, nuclear localization signal; RCC1, RCC1 homology and auto-inhibitory domain; “Gly”, polyglycine domain; “PXXP”, proline rich domain; *S/T P indicates a serine/threonine phosphorylation (P) site; “Nek6 binding”, Nek6 binding domain; “Coiled-coil”, homo-oliogomerization domain. See text for details.

FIGS. 2A-2C show results demonstrating that Nercc oligomerizes through its C-terminal coiled-coil domain.

FIG. 2A shows a Nercc coiled-coil prediction carried out with the Coils 2.1. software (window 28). The propensity of a sequence to form coiled-coils on a scale from 0 to 1 is plotted against the linear sequence of amino acids. The sequence (amino acids 891-940 of SEQ ID NO:2) of the predicted Nercc coiled-coil is shown; leucine residues (L) are shown in bold.

FIG. 2B shows results from a study in which HEK293 cells were transfected with HA Nercc and FLAG Nercc. The anti-HA immunoprecipitate was blotted with anti-FLAG antibody (upper panel); expression of the constructs is shown in the lower panel. See text for details.

FIG. 2C. Left panels, HEK293 cells were transfected with FLAG Nercc full length (FL) or FLAG Nercc (1-891) and either GST or a GST fusion to the Nercc coiled coil GST Nercc (891-940). GST-agarose isolates were blotted for FLAG (upper panel) or GST (middle panel); FLAG Nercc expression in cell lysates is shown in the lower panel. Right panels, FLAG Nercc (FL) or FLAG Nercc (1-891) were cotransfected with HA Nercc FL. The HA immunoprecipitates were blotted for FLAG (upper panel) or HA (middle panel); expression of Flag Nercc in cell lyastes is shown in the lower panel. IP, immunoprecipitated using indicated antibody. IB, immunoblotted using indicated antibody.

FIGS. 3A-3D show results demonstrating Nercc auto-activation in vitro.

FIG. 3A shows results from a study in which FLAG Nercc was immunoprecipitated from HEK293 cells, washed and incubated at 25° C. in phosphorylation buffer for the indicated times with 10 μM or 100 μM ATP. Incubations were terminated by washing, followed by the addition of 10 μM [³²P]ATP and histone H3 (1 μg/50 μl). After 10 minutes at 30° C., ³²P incorporation was stopped by addition of SDS sample buffer, followed by SDS-PAGE and blot transfer. Anti-FLAG immunoblot (upper panel), ³²P autoradiography (middle panel), and the relative quantity of ³²P incorporated into histone H3 (bottom panel) are shown.

FIG. 3B shows results from a study in which immobilized FLAG-tagged Nerec variants, isolated after transient expression in HEK293 cells, were washed and incubated in phosphorylation buffer for 30 min at 25° C. with Mg²⁺ with or without 100 μM ATP. After an additional wash, samples were incubated at 30° C. with Mg²⁺ plus 10 μM [³²P]ATP and histone H3 (1 μg/50 μl). After 10 min the reaction was stopped by addition of SDS sample buffer followed by SDS-PAGE and blot transfer. The ³²P autoradiogram (upper panel) and anti-FLAG immunoblot (middle panel) are shown.

FIG. 3C shows results from a time course of activation of the H3 kinase activity of wild type and mutant Nercc. FLAG Nercc (wild type, squares ▪), FLAG Nercc (Δ346-732, circles ●), FLAG Nercc (1-391, diamonds ♦) and FLAG Nercc (1-891, triangles ▴) were expressed in HEK293 cells, immobilized on anti-FLAG-agarose, washed and incubated at 25° C. with Mg²⁺ plus 100 μM ATP. At the times indicated, samples were washed, followed by addition of Mg²⁺ plus 10 μM [³²P]ATP and histone H3 (1 μg/50 μl). After 10 min at 30° C. SDS sample buffer was added, and ³²P incorporation into H3 was measured (using a PhosphorImager imaging system) after SDS-PAGE and blot transfer. ³²P incorporation is expressed as a percentage of Nercc wild type value at t=0, i.e. no preincubation with 100 μM ATP.

FIG. 3D shows results indicating that Nercc protein kinase domain and RCC1 domain interact in vivo. HEK293 cells were transfected with HA Nercc protein kinase domain (HA Nercc1-391) and either FLAG Nerco RCC1 domain (FLAG Nercc 338-778) or empty plasmid. Anti-FLAG immunoprecipitates were immunoblotted with anti-HA (upper panel) or anti-FLAG (middle panel). The expression of HA Nercc1-391 is shown in the lower panel. Diagrams of various Nercc constructs indicated under lower panel. See text for details.

FIG. 4 shows detection of Nek7 phosphorylation by Nercc using a phospho-specific antibody. Bacterial purified GST Nek7 was incubated with Mg²⁺/[γ-³²P]ATP with or without active FLAG Nerec (immunoprecipitated from HEK 293 cells and preactivated by incubation in 100 μM ATP). Lane 1, Nercc alone; lane 2, GST Nek7 plus Nercc; lane 3, GST Nek7 alone. Coomassie stain of the electrophoretical gel (upper panel), ³²P autoradiography (middle panel), and immunoblot using anti-phospho-Ser195 Nek7 (lower panel) are shown.

FIGS. 5A-5E show that Nercc kinase is activated during mitosis, and can be phosphorylated in vitro by p34^{Cdc2}.

FIG. 5A shows results indicating that Nercc displays a marked slowing in electrophoretic mobility (indicated by asterisk) during mitosis in a study in which HeLa cells were isolated in different phases of the cell cycle. G1/S, cells arrested with aphidicolin (2 μg/ml overnight); G2, cells arrested with aphidicolin and released for 6 hours; M, mitotic cells isolated by shake-off from a culture treated with nocodazole (500 ng/ml overnight). G1 mitotic cells, isolated as above, were washed repeatedly, replated, and harvested 6 hours thereafter. Exp., exponentially growing cells. Each cell cycle stage designation was confirmed by FACS. An immunoblot of endogenous Nercc (C1 antibody) at the different cell cycle stages is shown. See text for details.

FIG. 5B shows a slowing of electrophoretic mobility of Nercc on SDS-PAGE occurs during normal progression through mitosis. HeLa cells were partially synchronized using thymidine (2 mM thymidine overnight plus release). The resulting mitotic cells were collected by shake-off 9 hours later and compared to exponentially growing cells (Exp.), mitotic, nocodazole-arrested cells detached after mitotic shake-off (Noc. M), and nocodazole-treated cells that remain attached after shake-off (non-mitotic cells, Noc. Non-M); M, mitotic cells isolated by shake-off from a culture treated with nocodazole. Extracts of each cell type were subjected to immunoblot using anti-Nercc C1 antibody. See text for details.

FIG. 5C shows that Nerce kinase is activated in mitosis. Immunoprecipitations were carried out using extracts from nocodazole-treated cells that remained attached after shake-off (non-mitotic cells, Noc. Non-M), exponentially growing cells (Exp.), or mitotic nocodazole-arrested cells (Noc. M), with both preimmune rabbit IgG (NIgG) and affinity purified anti-Nercc antibody (N1). Mitotic cells (M) were cells that were not arrested with nocodazole, but obtained after release from a previous arrest at G1/S. Immunoprecipitates were washed sequentially with lysis buffer and phosphorylation buffer, and incubated for 10 minutes at 30° C. with Mg²⁺ plus [γ-³²P]ATP (10 μM) and histone H3 (2 μg/50 μl). The reaction was stopped by addition of SDS sample buffer. An anti-Nercc (N1) immunoblot of the immunoprecipitates is shown in the upper panel, and the ³²P incorporation into Nerec (middle panel) and histone H3 (lower panel) is shown. ³²P incorporation in NIgG immunoprecipitates (background) was quantified by PhosphorImager imaging system and subtracted from H3 ³²P incorporation in anti-Nerce immunoprecipitates. The resulting Nercc activity was expressed as percentage of activity in exponentially growing cells. Asterisk indicates slower electrophoretic mobility.

FIG. 5D shows Nerce activation during mitosis is due to phosphorylation. FLAG Nercc pre-activated by incubation with 100 μM ATP (filled bars), and endogenous Nerce immunoprecipitated from cells arrested in mitosis by nocodazole (open bars), were incubated in alkaline phosphatase buffer with no addition (“−”, bars 1 and 2) or with (“+”) 40 U of calf intestine alkaline phosphatase (“AP”, bars 3-6), without (“−”, bars 3, 4) or with (“+”, bars 5, 6) 4 mM EGTA. After washing, Nercc activity was assayed and expressed as a percentage of non-phosphatase treated enzyme (bars 1 and 2).

FIG. 5E shows that Nercc is an in vitro substrate for p34^{Cdc2}. FLAG Nercc (K81M) was produced in HEK293 cells, immunopurified with anti-FLAG antibody and eluted from the immunoprecipitates with FLAG peptide. Soluble K81M was incubated at 30° C. for the indicated times in phosphorylation buffer containing 100 μM [γ-³²P]ATP with and without purified active p34^{Cdc2}/Cyclin B from Xenopus (maturation promoting factor, MPF). Coomassie staining and ³²P autoradiography of Nercc K81M is shown. Quantitation of incorporated ³²P into Nercc K81M was carried out by PhosphorImager imaging system. Asterisk indicates slower electrophoretic mobility.

FIGS. 6A-6B show the effect of microinjection of anti-Nerce IgG into PtK2 cells.

FIG. 6A shows results in a time-lapse recording of a study in which PtK2 cells were microinjected with affinity purified anti-Nercc (C1) IgG (2.5 mg/ml; typically, the volume of microinjected material comprised approximately 10% of cell volume) in prophase. Representative phase contrast images from time-lapse recordings are shown. Recorded cells were fixed 3 minutes after the last image in sequence shown and stained with Hoechst 33342 DNA stain (lowest image in each panel). Time in minutes is shown in the right hand corner of the images, with acquisition of the last frame before the onset of anaphase serving as time “0” (A) and (B). The first image in panel (C) was taken 2 minutes after the nuclear envelope breakdown. Bar, 10 μm.

Panel (A): Anaphase A starts and proceeds normally, but the poles do not separate. Chromosomes remain trapped in the cytokinetic furrow and a “bridge” of DNA remains between the daughter cells.

Panel (B): An example of an extreme case of the absence of anaphase B phenotype. After moving the chromosomes apart in anaphase A, the substantial further separation typical of anaphase B does not occur, and a cytokinetic furrow separates the daughter cells into one containing all chromosomes and a cytoplast. Hoechst staining confirms the absence of DNA in the right cell.

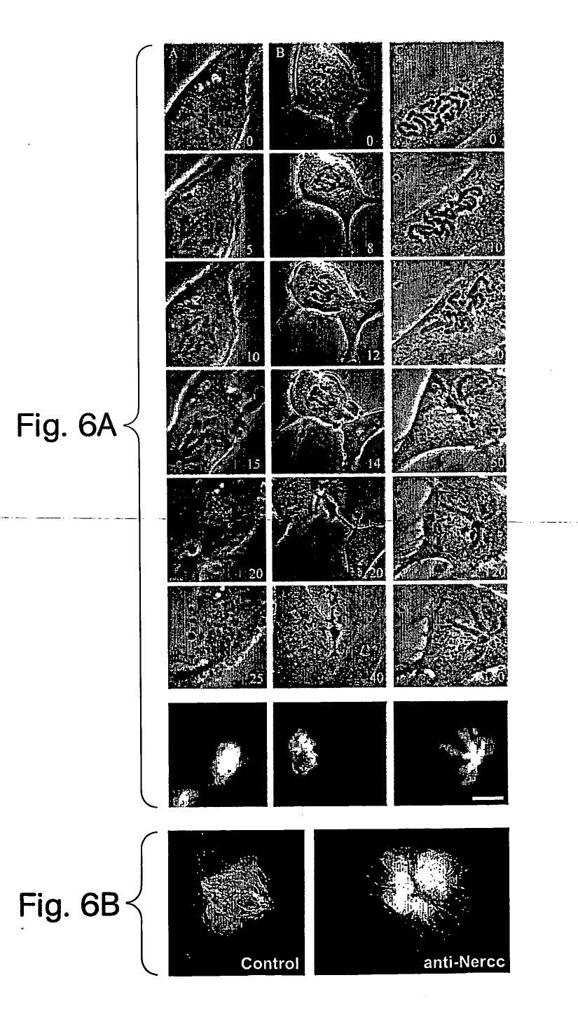

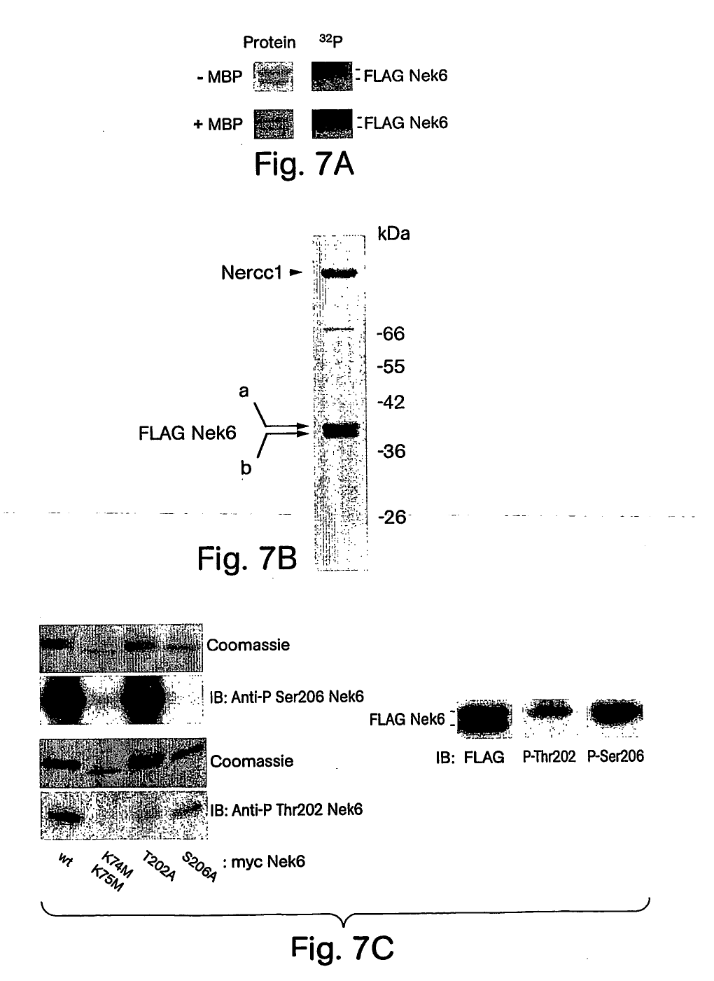

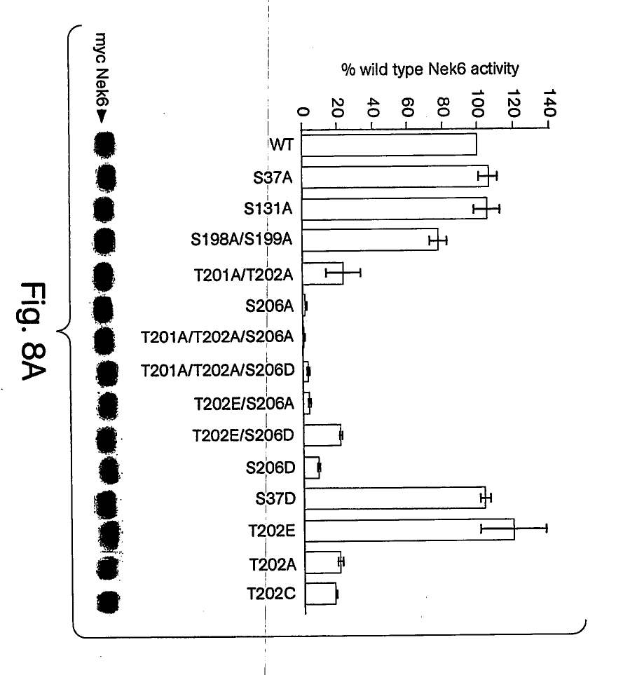

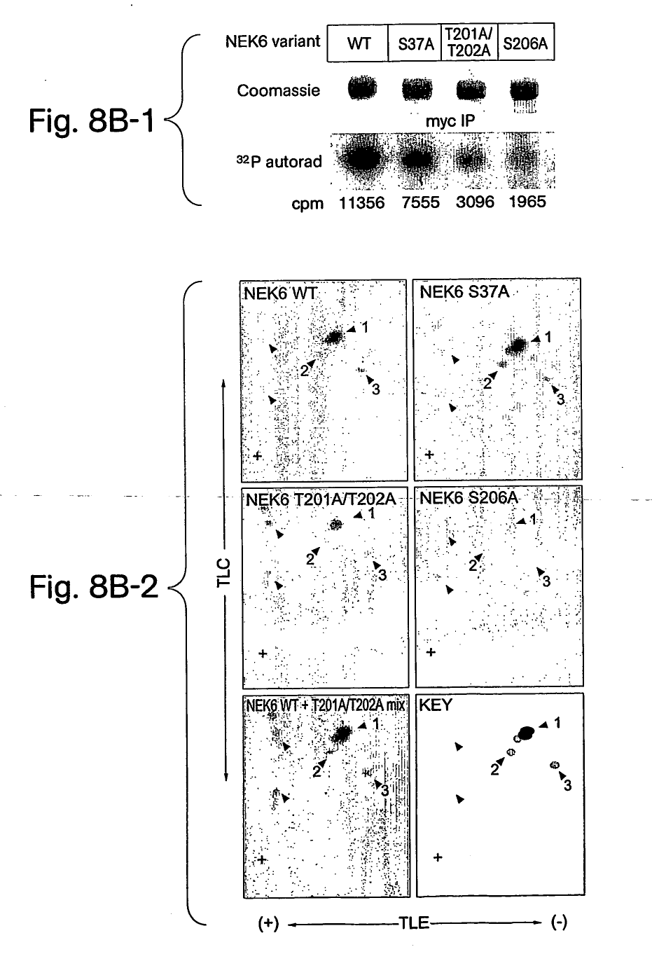

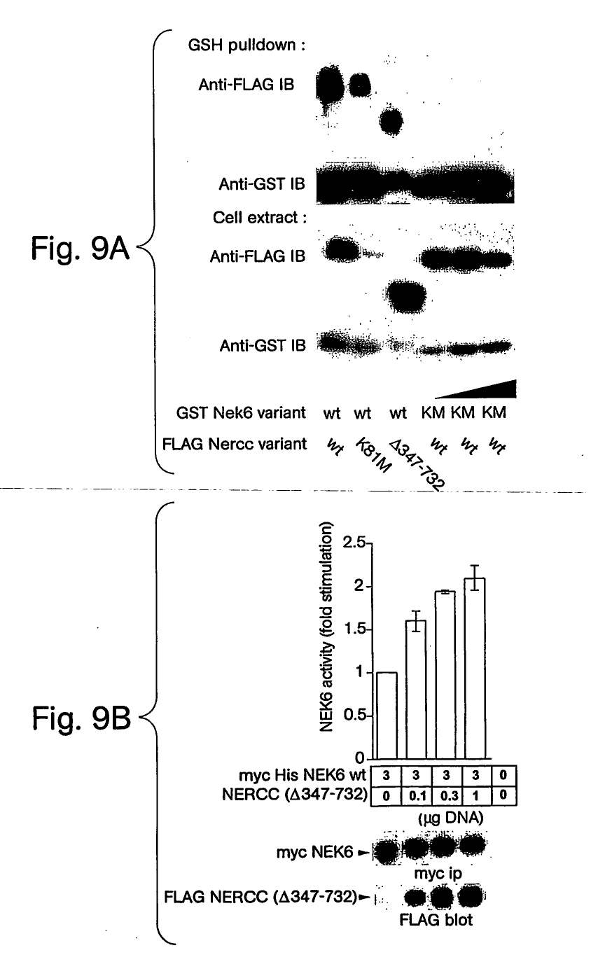

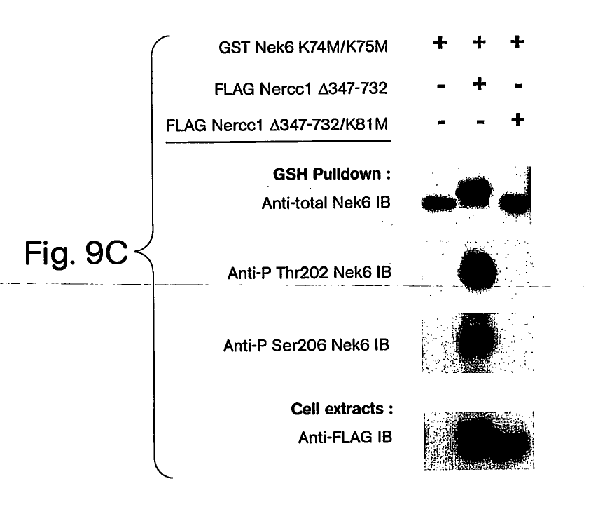

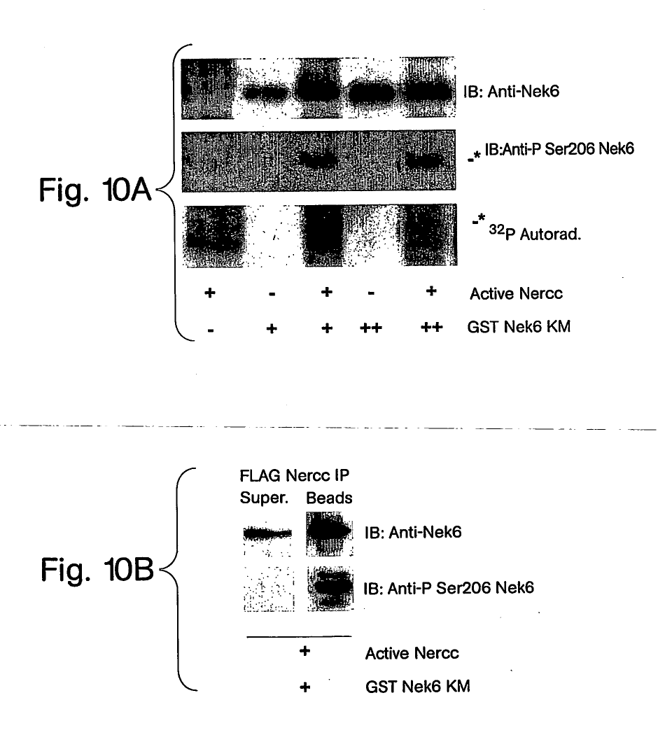

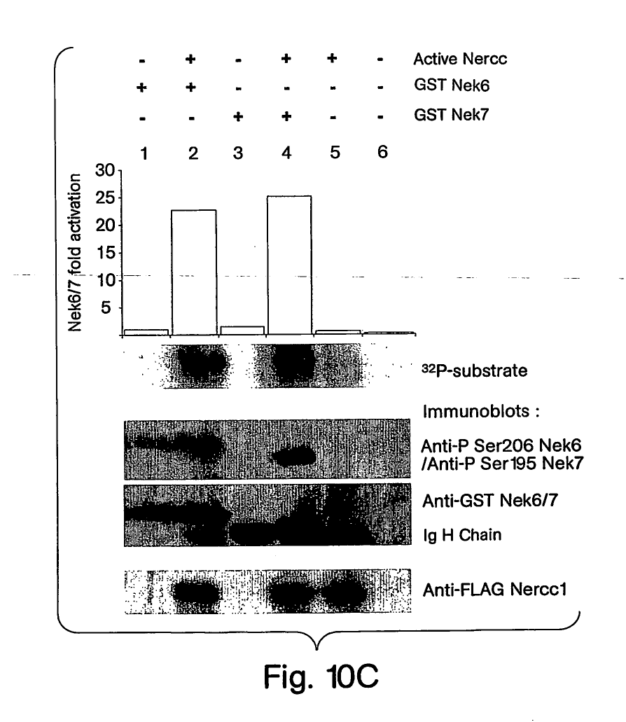

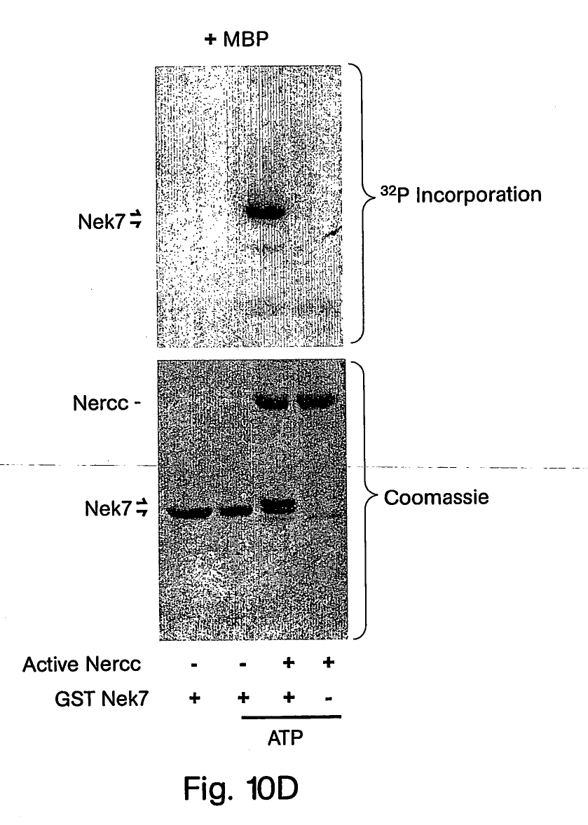

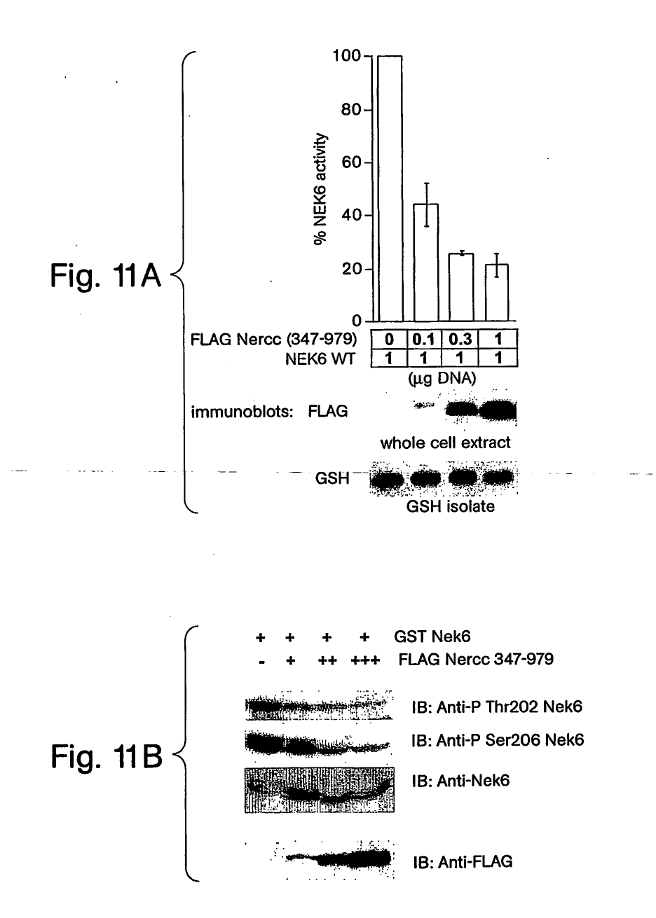

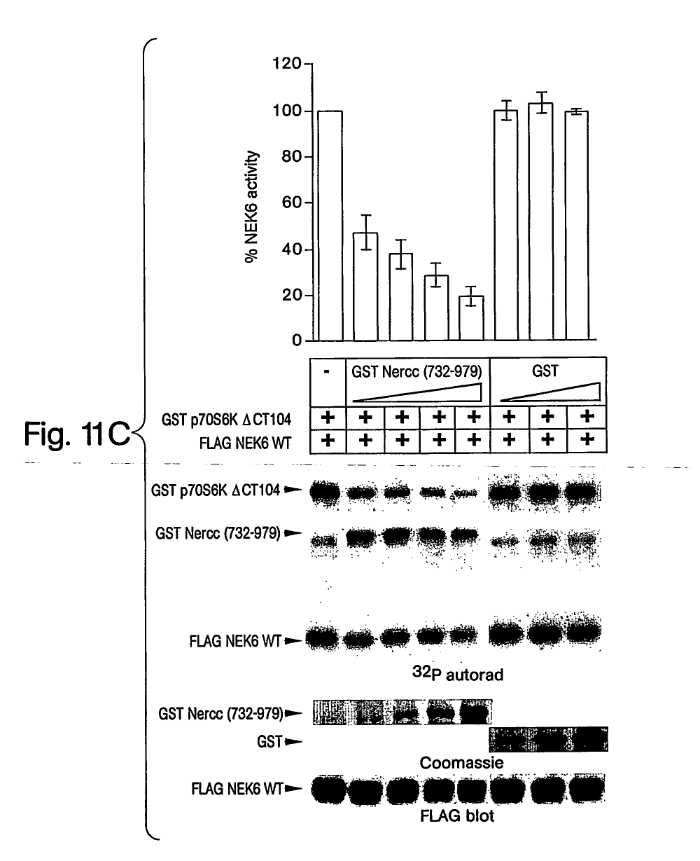

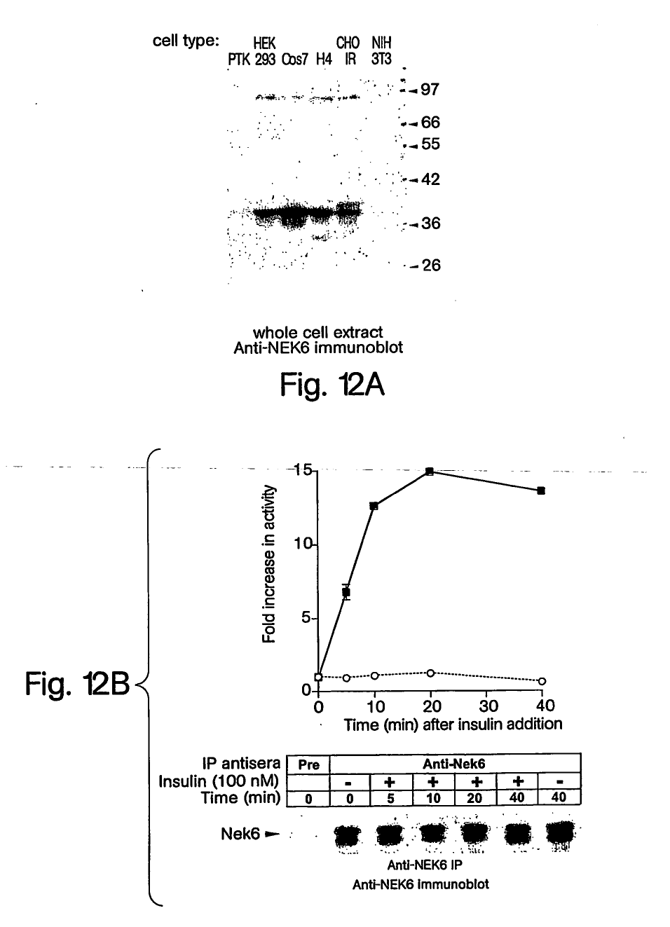

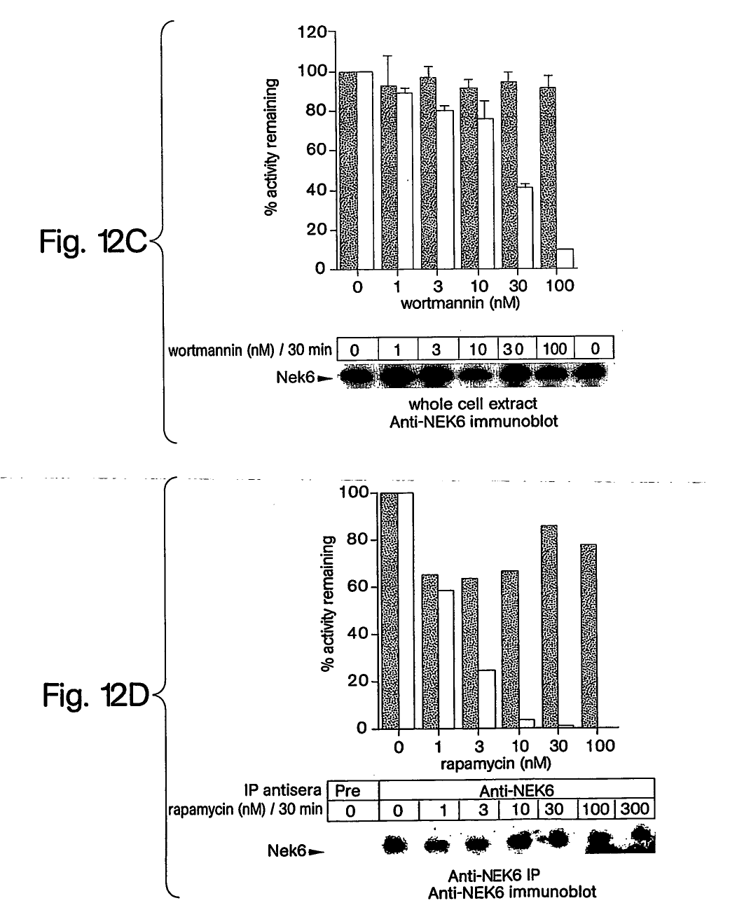

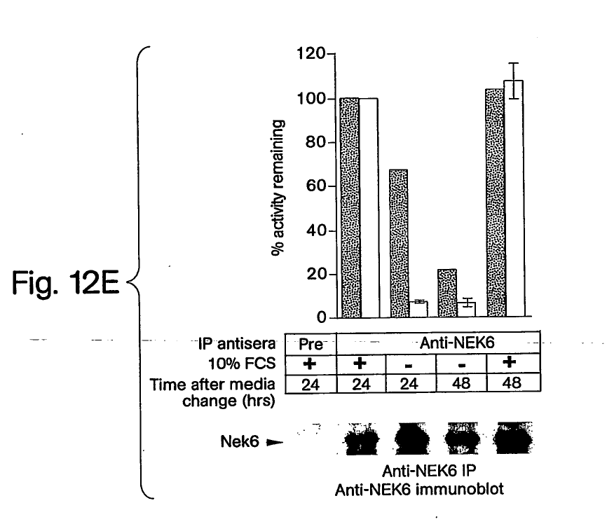

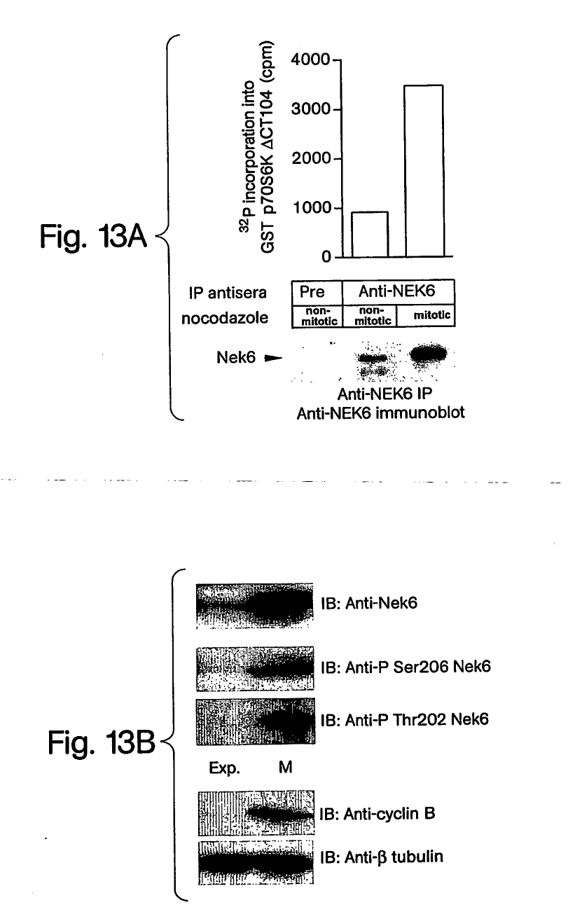

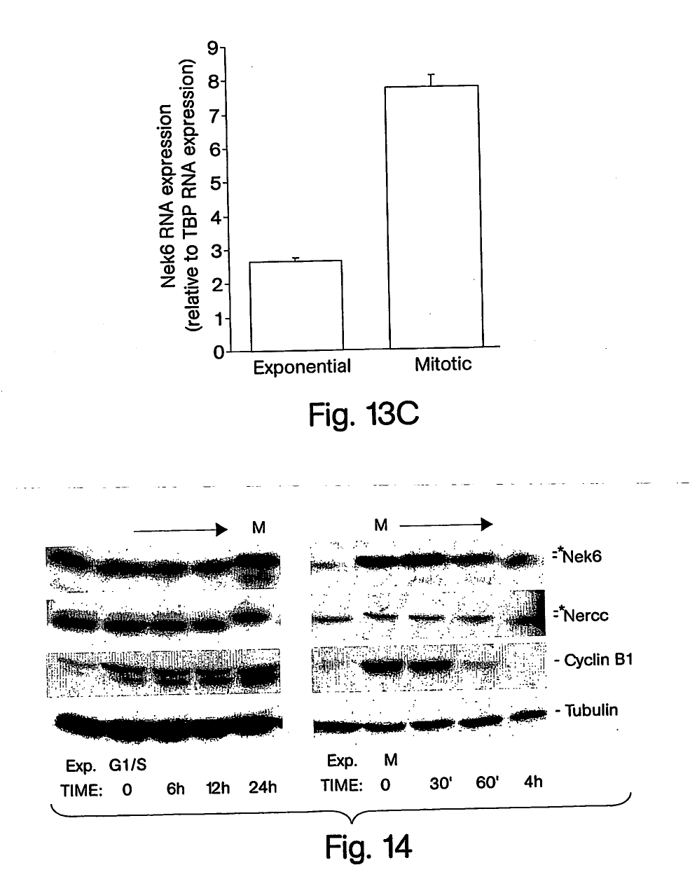

Panel (C): After the nuclear envelope breakdown, the cell fails to form a mitotic spindle, or the spindle collapses soon after formation. Mitotic progression stops in prometaphase. See, FIG. 6B.

FIG. 6B shows a comparison in which Ptk2 cells were microinjected with normal IgG (control) or anti-Nercc (C1) IgG (anti-Nercc) in prophase. Cells were fixed and stained with Hoechst 33342 DNA, and anti-tubulin antibody (control cells were fixed at metaphase; anti-Nercc injected cells failed to enter a normal metaphase, and were fixed at t=120 minutes after microinjection). See text for details.

FIGS. 7A-7C show that active Nek6 is phosphorylated at amino acid residues Ser37, Thr202, and Ser206 in intact cells.

FIG. 7A shows results of in-gel kinase assay of FLAG Nek6 immunoprecipitates. FLAG Nek6 was immunoprecipitated from HEK293 cells, washed with lysis buffer, and boiled in SDS sample buffer for 10 minutes. The sample was divided in two, and each aliquot electrophoresed in a 10% acrylamide SDS-PAGE gel polymerized without (upper panels) or with (lower panels) MBP. The in-gel linase assay was carried out as described in Example 12. The gels were fixed, stained with Coomassie, and autoradiographed. Protein stain (left) and ³²P autoradiography (right) are shown.

FIG. 7B shows that recombinant FLAG Nek6 is expressed as a doublet and binds endogenous Nercc1. FLAG Nek6 polypeptide, transiently expressed in HEK293 cells, was immunoprecipitated with anti-FLAG, washed and subjected to SDS-PAGE; the Coomassie-blue stained gel is shown. Bands a and b, each corresponding to FLAG Nek6, were excised, digested in situ with trypsin, and analyzed by LC/MS/MS.

FIGS. 7C shows detection of phosphorylated Nek6 with phosphospecific antibodies. Left panel: The specificity of the antibodies raised against phosphopeptides (P) containing either Nek6 P-Thr202 or Nek6 P-Ser206 was tested by immunoblot of myc Nek6 wild type, Nek6 (S206A) or Nek6 (S202A), immunoprecipitated after transient expression in HEK293 cells. Right panel: Immunoblot using the different phosphospecific antibodies of a cell extract from HEK293 transfected with a vector expressing FLAG Nek6 wild type (wt).

FIGS. 8A-8B show characteristics and properties of mutant variant Nek6 proteins compared to wild type Nek6 protein.

FIG. 8A shows the effect of site-specific mutations on the activity of Nek6. The activity of Nek6 and of a variety of Nek6 site-specific mutants, each containing a carboxy (C) terminal myc/6His epitope, were examined after transient expression in HEK293 cells and anti-myc immunoprecipitation. Kinase assays were performed as described in Example 12 using Mg²⁺[γ³²-P]ATP and purified recombinant GST p70S6K ΔCT104(1252A) polypeptide as the substrate for Nek6-mediated phosphorylation. The reaction mixture was subjected to SDS PAGE, transfer to PVDF membrane and the Coomassie-stained bands corresponding to GST p70S6K ΔCT104 Thr252Ala were excised and their ³²P content determined. Kinase activities of mutant Nek6 polypeptides are depicted by the histogram as the mean±S.E.M percent of the activity of wild type enzyme assayed in parallel from 3-5 observations for each Nek6 variant. A composite anti-myc immunoblot is shown, normalized for the expression of the wild type Nek6, which was included in each experiment.

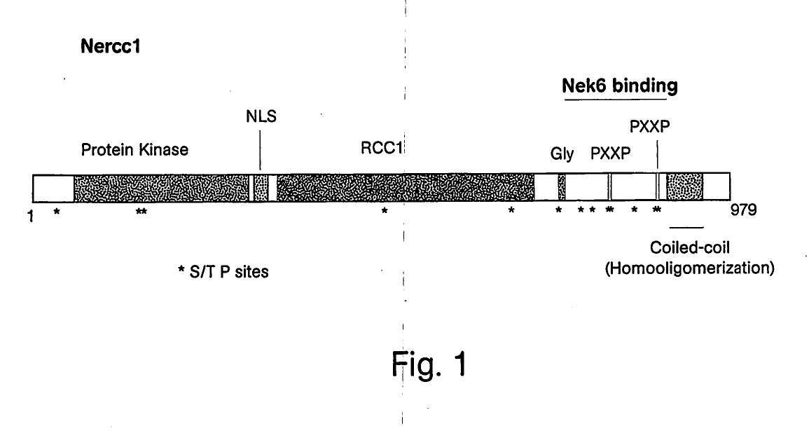

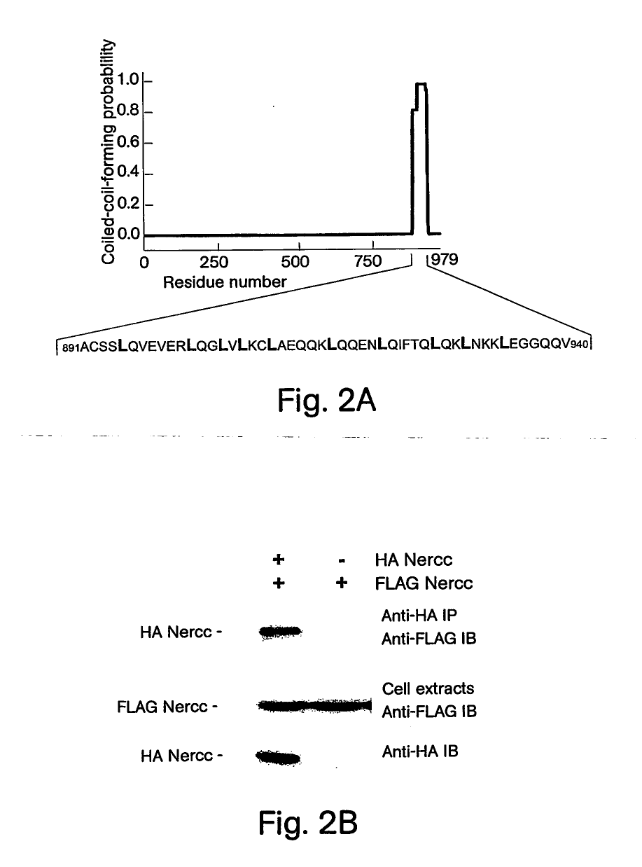

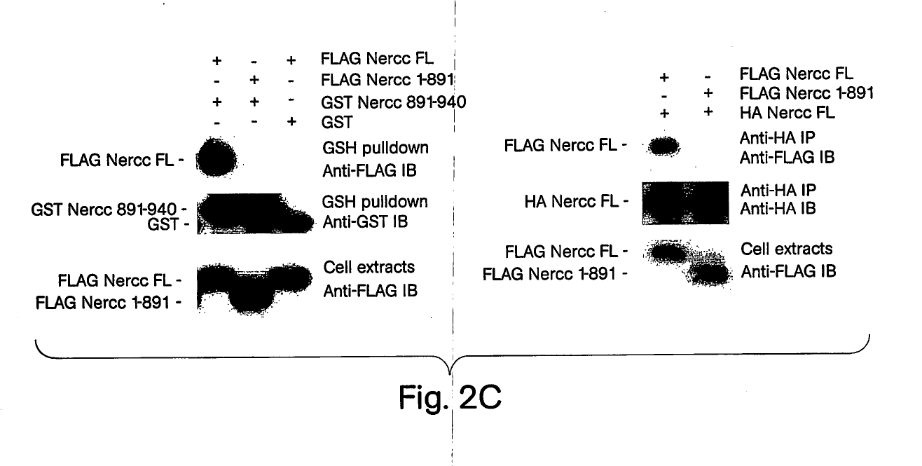

FIG. 8B shows two-dimensional tryptic phosphopeptide mapping of Nek6. FIG. 8B-1: HEK293 cells, transiently expressing myc-epitope-tagged Nek6 wild-type, Nek6 (S37A), Nek6 (T201A/T202A), or Nek6 (S206A), were incubated for 4 hours with phosphate-free DMEM containing ³²P-orthophosphate. ³²P-labelled recombinant Nek6 variants were immunoprecipitated and resolved by SDS-PAGE. Gels were stained with Coomassie blue (see, upper part of Panel a) and exposed to autoradiography (see, lower part of Panel a) prior to excision of stained polypeptide bands from gels for Cerenkov counting and digestion with trypsin in situ. FIG. 8B-2: Aliquots of digests containing equal ³²P-cpm were subjected to thin-layer electrophoresis (TLE) followed by thin layer chromatography (TLC) (see text for details). Plates were exposed in a Phosphoimager. The maps for Nek6 wild type (top left of Panel b) and Nek6 (T201A/T202A) (top right of Panel b), Nek6 (S37A) (middle left of Panel b), and Nek6 (S206A) (middle right of Panel b) mutants are shown. A mixture containing equal cpm of wild type and Nek6 (T201A/T202A) digests was also subjected to TLE/TLC (bottom left of Panel B). The lower right panel depicts a cartoon of the Nek6 wild type two-dimensional map identifying the major radioactive spots (labelled 1-3). The origin of sample application is marked with a cross, +.

FIGS. 9A-9C show that Nercc1 binds and activates Nek6 in vivo.

FIG. 9A shows Nek6 binding to Nercc1. Vectors encoding wild type (“wt”) or kinase-inactive GST Nek6 (K74M/K75M) (“KM”) were cotransfected in HEK293 cells with wild type (“wt”) or mutant (“K81M”, “Δ347-732”) versions of FLAG Nercc1. Extracts were incubated with GSH-agarose; aliquots of the cell extracts and of the SDS eluates from well-washed beads were subjected to SDS-PAGE and immunoblot using the antibodies indicated.

FIG. 9B shows that cotransfection with constitutively active Nercc1 increases the activity of coexpressed Nek6 activity. HEK293 cells were transfected with myc 6His Nek6 alone or in combination with increasing amounts of FLAG Nercc1 (Δ347-732) and deprived of serum 24 hours prior to harvest. The linase activity of recombinant Nek6 immunoprecipitated from cell lysates was measured using GST p70S6K ACT104(T252A) as substrate; the latter is not a substrate for Nercc1.

FIG. 9C shows that a constitutively active mutant variant of Nercc1 causes phosphorylation of the Nek6 (K74M/K75M) activation loop in vivo. HEK293 cells were transfected with GST Nek6 (K74M/K75M) alone or together with FLAG Nercc1(Δ347-732) or FLAG Nercc1 (Δ347-732) (K81M). GST Nek6 (K74M/K75M) was isolated by adsorption to GSH-agarose. Each eluate (GSH pulldown) from the washed beads was divided into three samples and subjected to immunoblot using an antibody specific for total Nek6 polypeptide (top panel), or the anti-Nek6 phosphospecific antibodies: anti-phospho(P) Thr202 Nek6 (second from top) and anti-P Ser206 Nek6 (third from top). An immunoblot of the cell extracts using anti-FLAG antibody is shown in the lowest panel indicating the presence of a FLAG Nercc protein.

FIGS. 10A-10D show that Nercc1 phosphorylates and activates Nek6/7 in vitro.

FIG. 10A shows results when GST Nek6 (K74M/K75M) (i.e., “GST Nek6 KM”) was isolated from transfected HEK293 cells on GSH-agarose, washed and eluted with glutathione, and dialyzed overnight against TBS plus protease inhibitors. 0.5 μg (+) or 1 μg (++) of the isolated GST Nek6 protein were incubated for 30 minutes with [γ-³²P]ATP in the presence or absence of preactivated (by incubation with 100 μM ATP+Mg²⁺ for 20 minutes, followed by washing) FLAG Nercc1, immobilized on protein A agarose beads. The reaction mixture was separated by SDS-PAGE and subjected to immunoblot with anti-total Nek6 (top panel), anti-phospho(P) Ser206 Nek6 (middle panel), and autoradiography (lower panel).

FIG. 10B shows that only GST Nek6 (K74M/K75M) (i.e., “GST Nek6 KM”) bound to Nercc1 exhibited Ser206 phosphorylation. As in FIG. 10A, except that nonradioactive ATP was employed, and the beads containing ProteinA-FLAG Nercc1 were sedimented and washed prior to elution into SDS. The Nercc1 beads (Beads) and the reaction supernatant (Super.) were subjected to SDS-PAGE and immunoblot with anti-Nek6 polypeptide (top panel) or anti-phospho(P) Ser206 Nek6 (middle panel) antibodies.

FIG. 10C shows that GST Nek6 and GST Nek7 were expressed in bacteria and purified on GSH-agarose. After elution with glutathione, Nek6 and Nek7 were incubated with and without preactived (by incubation with 100 μM ATP for 20 minutes) FLAG Nercc1 immobilized on Protein A-agarose and nonradioactive ATP. After 30 minutes, [γ-³²P] ATP was added together with GST Cdc16 Ct, a Nek6/7 substrate. The reaction was stopped after a further 30 minutes by addition of SDS. After SDS-PAGE, ³²P incorporated into the GST Cdc16 Ct substrate (second panel from top) was quantified using a PhosphorImager imaging system and is shown as a histogram in the top panel. Immunoblots of Nek6 P Ser206/Nek7 P Ser195, GST Nek6/7 and FLAG Nercc1 are shown below.

FIG. 10D shows results when GST Nek7 was incubated with preactived FLAG Nercc1 as described above, except the GST Cdc16Ct and [γ-³²P] ATP were omitted. The reaction mixture was analyzed by an “in-gel” kinase assay, using MBP as substrate, as described in the text. ³²P autoradiography (top) and the Coomassie stain (lower) of the gel are also shown.

FIGS. 11A-11C show that the Nercc1 non-catalytic domain inhibits Nek6 activation in vivo and activity in vitro.

FIG. 11A shows that Nek6 kinase activity is inhibited by overexpression of a Nercc1 variant lacking the N-terminal kinase domain in vivo. GST Nek6 was coexpressed with increasing quantities of FLAG Nercc1 (347-979). The kinase activity of GST Nek6 was measured using MBP as substrate.

FIG. 11B shows that Nercc1 (347-979) suppresses Nek6 activation loop phosphorylation in vivo. GST Nek6 was cotransfected in HEK293 cells with increasing amounts of FLAG Nercc1 (347-979), and immunoblots of the extracts were carried out using the indicated antibodies.

FIG. 11C shows that Nercc1 (347-979) inhibits Nek6 activity in vitro. Transiently expressed FLAG Nek6 was incubated for 1 hour at 4° C. alone or with increasing quantities of either purified prokaryotic recombinant GST (lanes 6-8) or prokaryotic recombinant GST Nercc1 (732-979) (lanes 2-5). Nek6 activity was assayed using p70S6K ΔCT104 as substrate. Upper panel: mean±S.E.M. percent of control Nek6 activity. Lower panels: ³²P autoradiography, Coomassie stain of the GST Nercc1 (732-979) or GST proteins, and western blot of the FLAG Nek6 wild type.

FIGS. 12A-12E demonstrate the regulation of endogenous Nek6 activity FIG. 12A shows an anti-Nek6 immunoblot of cell extracts of PTK, HEK293, COS7, H4-II-E-C3, CHO-IR and NIH3T3 cells, normalized for total protein.

FIGS. 12B-12D show that Nek6 activity endogenous to H4-II-E-C3 hepatocytes is not altered by agents that modify p70 S6 kinase activity. Endogenous Nek6 polypeptide was immunoprecipitated from lysates of rat H4-II-E-C3 hepatoma cells following prior treatment for varying times (min) with 100 nM insulin (FIG. 12B), or insulin plus increasing concentrations of either wortmannin (FIG. 12C) or rapamycin (FIG. 12D) for 30 minutes. Nek6 kinase activity was measured using GST p70S6K ΔCT104 (Thr252Ala) as substrate (see, text for details). The activity of endogenous p70S6K αI/II, immunoprecipitated from the same lysates, was measured using a synthetic peptide substrate. Kinase activities are expressed as either fold increase in baseline activity (as in FIG. 12B; open circles show Nek6 activity; filled squares show p70S6K activity) or as histograms depicting the percent activity remaining for Nek6 (filled bars) or p70S6K (open bars) (as in FIGS. 12C and 12D).

FIG. 12E shows that endogenous Nek6 activity is reduced by serum (FCS) withdrawal. Complete media maintaining exponentially growing H4-H-E-C3 cells was replaced at time 0 hrs with fresh media either with (+) or without (−) serum. Cells were harvested at 24 and 48 hours thereafter. Both-immunoprecipitated endogenous Nek6 and p70S6K αI/II activities were measured as above.

FIGS. 13A-13C show Nek6 synthesis and activation in mitosis.

FIG. 13A shows Nek6 abundance and activity increase in mitosis. Exponentially growing H4-II-E-C3 cells were treated with 500 ng/ml of nocodazole for 16 hours. Thereafter, culture plates were lightly shaken to release the loosely attached (non-adherent) mitotic cells, which were harvested by centrifugation of the cell culture supernatant. Cell lysates were prepared from both the pool enriched with mitotic (non-adherent) and non-mitotic interphase (adherent) cells, and the relative abundance (by immunoblot) and activity of endogenous Nek6 (after immunoprecipitation) were determined.

FIG. 13B shows Nek6 protein levels are increased and Nek6 Thr202 and Ser206 are phosphorylated in mitotic HeLa cells. Extracts prepared from HeLa cells exponentially growing (Exp.) or arrested in mitosis by 500 ng/ml nocodazole and isolated by mitotic shake off (M) were subjected to SDS-PAGE and immunoblot using the indicated antibodies.

FIG. 13C shows Nek6 mRNA expression is increased in mitotic HeLa cells. RT-PCR quantification of Nek6 mRNA levels from exponentially growing and mitotic HeLa cells (nocodazole arrested in mitosis and isolated as above) was carried out as described in Materials and Methods. Nek6 RNA amount relative to TBP RNA amount is shown. Average±S.E.M. of three different experiments is shown.

FIG. 14 shows the results from a time course experiment for synthesis and activation of Nercc1 and Nek6 in mitosis. The abundance and electrophoretic mobility of Nek6 was determined in human U2OS cells in different phases of the cell cycle by western blot using specific antibodies, in parallel to that of Nercc 1. Cyclin B1 was determined by western blot as a mitotic marker, while tubulin was used as a loading control. Left panel: entry (arrow) into mitosis (M) after G1/S arrest. Exp., exponentially growing cells; G1/S (t=0), cells arrested in G1/S by 2 μg/ml aphidicolin for 6-24 hours, thereafter cells were released from the aphidicolin block into media containing 500 ng/ml nocodazol. Right panels: exit (arrow) from mitosis (M) after a nocodazol arrest. Exp., exponentially growing cells; M (t=0), mitotic cells arrested with 500 ng/ml nocodazol; 30 minutes, 60 minutes, 4 hours, cells released from the nocodazol block. Asterisk (*) indicates electrophoretic migration of phosphorylated (i.e., activated) form of Nek6 or Nercc1.

### DETAILED DESCRIPTION OF THE INVENTION

The invention is based on the discovery of a specific cascade of kinase proteins involved in the regulation of mitosis in eukaryotic cells. In particular, the NIMA-like kinases Nercc1 kinase, Nek6, and its homolog Nek7 play critical roles in a cascade of kinases that signals eukaryotic cells to enter and/or maintain mitosis. According to the invention, Nercc1 kinase (also referred herein as “Nercc1”, “Nercc kinase”, “Nercc”, Nek9) activates Nek6 and Nek7 proteins by phosphorylating these proteins at specific proline-rich, serine/threonine phosphorylation sites (“S/T P sites”). Activated Nek6 or Nek7 proteins in turn phosphorylate a subsequent member in the cascade to direct a eukaryotic cell to enter or maintain mitotic progression. Like Nek6 and Nek7, Nercc1 kinase must also be phosphorylated at one or more specific S/T P sites to be an active kinase, however, unlike Nercc1 is also able to carry out auto-phosphorylation to effect auto-activation. Described herein are compositions and methods for compounds that inhibit mitosis by specifically inhibiting one or more of these NIMA-like kinases that are critical to mitotic progression of eukaryotic cells.

Undesirable entry into and/or maintenance of mitotic progression by eukaryotic cells are characteristic of most cancers, especially aggressive malignant and/or metastatic tumors. Accordingly, the ability to identify new inhibitors of mitosis (i.e., “anti-mitotic compounds”), which inhibit or interrupt mitosis by acting at particular molecular targets, such as a specific kinase, are highly desired as new agents to treat cancer as well as other conditions characterized by undesirable mitotic progression. Anti-mitotic compounds may thus also be used to treat various microbial infections. For example, new anti-mitotic compounds are highly sought after to prophylactically or therapeutically treat eukaryotic microbial infections, such as fungal infections, which can range from minor, but persistant surface (e.g., nail, skin) infections to life-threatening diseases (e.g., infections of the lungs, heart, endothelial tissue) as in the case of immuno-compromised individuals, such as HIV-infected patients and patients undergoing chemotherapy.

In order that the invention may be more fully understood, the following terms are defined.

“Antibody” or “antibody molecule”, as used and understood herein, refers to a specific binding member that is a protein molecule or portion thereof or any other molecule, whether produced naturally, synthetically, or semi-synthetically, which possesses an antigenic binding domain formed by an immunoglobulin variable light chain region or domain (V_{L}), or portion thereof, and/or an immunoglobulin variable heavy chain region or domain (V_{H}), or portion thereof. The term “antibody” also covers any polypeptide or protein molecule that has an antigen-binding domain that is identical, or homologous to, an antigen-binding domain of an immunoglobulin. Antibodies may be “polyclonal”, i.e., population of antigen-binding molecules that bind to different sites on the antigen or “monoclonal”, i.e., a population of antigen-binding molecules that bind to only one site on an antigen. Examples of an antibody molecule, as used and understood herein, include any of the well known classes of immunoglobulins (e.g., IgG, IgM, IgA, IgE, IgD) and their isotypes; fragments of imnmunoglobulins that comprise an antigen binding domain, such as Fab or F(ab′)₂ molecules; single chain antibody (scFv) molecules; double scFv molecules; single domain antibody (dAb) molecules; Fd molecules; and diabody molecules. Diabodies are formed by association of two diabody monomers, which form a dimer that contains two complete antigen binding domains wherein each binding domain is itself formed by the intermolecular association of a region from each of the two monomers (see, e.g., Holliger et al., Proc. Natl. Acad. Sci. USA, 90: 6444-6448 (1993)). Antibodies that specifically bind phosphorylated forms of a polypeptide or protein (i.e., “phospho-specific antibodies”) may be readily produced by standard methods, e.g., by immunizing rabbits with synthetic phophospeptides coupled to a carrier protein such as keyhole limpet hemocyanin (see, e.g., Weng et al., J. Biol. Chem., 273: 16621-16629 (1998)). Antibodies maybe employed in a variety of methods and formats that are well known in the art and/or that are commercially available. Such methods and formats include those described herein and include, but are not limited to, immunoblotting (e.g., western blotting), immunoprecipitation, fluorescent antibody cell sorting (FACS), enzyme-linked immunosorbent assay (ELISA), microinjection of antibodies into cells, affinity chromatography, and antibody cell surface labeling methods. Such methods and formats may be carried out manually, semi-automatically, or essentially automatically (robotically). Accordingly, one, several, or multiple (e.g., tens, hundreds, or even thousands) samples may be effectively processed by such antibody-based methods and formats.

As used herein “mitosis” is the complex process in a cell that apportions chromosomes equally to daughter cells as generally known in the art. Generally recognized phases of mitosis are prophase, metaphase, anaphase and telophase, which includes separation into separate daughter cells (cytokinesis). Uncontrolled mitotic progression of cells is a characteristic of cancer and is the basis of formation of all tumors, including those classically categorized as “benign” and “malignant”.

The term “phosphorylation” has the meaning known in the art, i.e., the term refers to a phosphate transfer in which a phosphate group from a donor molecule is transferred to an acceptor molecule. Preferred phosphate donor molecules useful in the invention include adenosine triphosphate (ATP) and guanosine triphosphate (GTP). Enzymes that mediate phosphorylation of acceptor molecules are referred to as kinases. Nercc1 linase is a kinase that is able to mediate phosphorylation of certain polypeptide substrates. A protein substrate for one kinase may itself be a kinase, which becomes activated upon phosphorylation. For example, inactive forms of Nek6 and Nek7 kinases are “activated” by phosphorylation at certain serine or threonine phosphorylation sites (“S/T P sites”) in the protein by Nercc1 kinase. Nercc1 kinase also must be phosphorylated at one or more specific S/T P sites to be active, however, Nercc1 kinase is able to “auto-phosphorylate” at such critical sites in the protein to be active (i.e., auto-activation). As described herein, such Nercc1 kinase auto-phosphorylation readily occurs in vitro in a reaction mixture comprising non-activated (non-phosphorylated) Nercc1 kinase and a purine nucleoside triphosphate, e.g., ATP or GTP.

The terms “polypeptide” or “protein”, as used herein, comprise a linear polymer of two or more amino acid residues linked by peptide bonds. The term “peptide” is used herein to refer to relatively short polypeptides, especially polypeptide having 20 or fewer amino acids. “Protein” may be synonymous with a single “polypeptide” or “peptide” or may comprise more than one “polypeptide” or “peptide” as in a dimeric or other multimeric protein. For example, active Nercc1 linase protein is typically a dimeric protein. Amino acids and amino acid sequences of polypeptides may be indicated using standard one or three letter abbreviations.

“Recombinant” refers to the result of methods, reagents, and laboratory manipulations in which nucleic acids or other biological molecules have been enzymatically, chemically, or biologically cleaved, synthesized, combined, or otherwise manipulated in at least one step in vitro in order to produce a desired product in cells or other biological systems. For example, a particular polynucleotide can be inserted into a suitable vector, such as a plasmid vector, and the resulting “recombinant” plasmid used to transform a suitable host cell to produce a “recombinant” protein encoded by the particular polynucleotide in the recombinant plasmid vector. The transformed host cell (“recombinant cell”) may be a prokaryotic or eukaryotic cell, including but not limited to, a bacterial, yeast, insect, and mammalian cell. Thus, “recombinant DNA” refers to a DNA molecule that is produced by such techniques, and a “recombinant host cell” refers to a cell that comprises a recombinant nucleic acid molecule, typically a recombinant plasmid or other expression vector. Recombinant host cells may express one or more gene products encoded on a recombinant nucleic acid molecules present in the recombinant cells. A “recombinant” protein is a protein expressed from a recombinant nucleic acid encoding the protein.

“Naturally occurring” refers to any substance such as an amino acid, nucleotide, nucleic acid, or protein that exists in nature without human intervention. If a naturally occurring substance is produced by human intervention, the resulting substance is referred to as “synthetic” or “recombinant.”

“Identity”, as used herein in reference to sequence alignment data bases refers to the subunit sequence similarity between two polymeric molecules, e.g., two peptide or two nucleic acid molecules. When a subunit position in both of the two molecules is occupied by the same monomeric subunit, e.g., if a position in each of two peptides is occupied by the same amino acid, then they are identical at that position. The identity between two sequences is a direct function of the number of matching or identical positions. For example, if half of the corresponding positions in two peptide or compound sequences are identical, then the two sequences are 50% identical; if 90% of the positions, e.g., 9 of 10, are matched, the two sequences share 90% sequence identity.

Various computer programs and databases are now available in the art for comparing and analyzing nucleotide and amino acid sequences.

The percent identity may be determined for any sequence of nucleotides or amino acids, for example, by comparing sequence information using the GAP computer program, version 6.0 described by Devereux et al. (Nucl. Acids Res., 12: 387 (1984)) and available from the University of Wisconsin Genetics Computer Group (UWGCG). The GAP program utilizes the alignment method of Needleman and Wunsch (J Mol. Biol., 48: 443 (1970)), as revised by Smith and Waterman (Adv. Appl. Math., 2: 482 (1981)). The preferred default parameters for the GAP program may include: (1) a comparison matrix (containing a value of 1 for identities and 0 for non-identities) for nucleotides, and the weighted comparison matrix of Gribskov and Burgess (Nucl. Acids Res., 14: 6745 (1986)), as described by Schwartz and Dayhoff (in Atlas of Protein Sequence and Structure, Schwartz and Dayhoff, eds., National Biomedical Research Foundation, pp. 353-358 (1979)); (2) a penalty of 3.0 for each gap and an additional 0.10 penalty for each symbol in each gap; and (3) no penalty for end gaps.

The comparison of sequences and determination of percent identity between two sequences can be accomplished using a mathematical algorithm. The percent identity between two amino acid sequences is determined using the GAP program in the GCG software package (available at www.geg.com), e.g., using either a Blossom 62 matrix or a PAM250 matrix, and a gap weight of 16, 14, 12, 10, 8, 6, or 4 and a length weight of 1, 2, 3, 4, 5, or 6. In yet another example, the percent identity between two nucleotide sequences may be determined using the GAP program in the GCG software package (available at www.gcg.com , using a NWSgapdna.CMP matrix and a gap weight of 40, 50, 60, 70, or 80 and a length weight of 1, 2, 3, 4, 5, or 6.

In another example, the nucleic acid and protein sequences of the present invention may further be used as a “query sequence” to perform a search against public databases to, for example, identify other family members or related sequences. Such searches can be performed using the NBLAST and XBLAST programs (version 2.0) of Altschul et al. (J. Mol. Biol., 215: 403-410 (1990)). BLAST nucleotide searches may be performed with the NBLAST program, score=100, wordlength=12 to obtain nucleotide sequences homologous to particular nucleic acid molecules of the invention. BLAST protein searches may be performed, e.g., with the XBLAST program, score=50, wordlength=3 to obtain amino acid sequences homologous to particular protein molecules of the invention. To obtain gapped alignments for comparison purposes, Gapped BLAST may be utilized as described in Altschul et al., Nucleic Acids Res., 25(17): 3389-3402 (1997). When utilizing BLAST and Gapped BLAST programs, the default parameters of the respective programs (e.g., XBLAST and NBLAST) may be used (as provided at www.ncbi.nlm.nih.gov).

As used herein the term “homology” refers to comparisons between protein and/or nucleic acid sequences and is evaluated using any of the variety of sequence comparison algorithms and programs known in the art. Such algorithms and programs include, but are by no means limited to, TBLASTN, BLASTP, FASTA, TFASTA, and CLUSTAL W (see, e.g., Pearson and Lipman, Proc. Natl. Acad. Sci. USA, 85(8): 2444-2448 (1988); Altschul et al., J. Mol. Biol., 215(3): 403-410 (1990); Thompson et al., Nucleic Acids Res., 22(2):4673-4680 (1994); Higgins et al., Methods Enzymol., 266: 383-402 (1996); Altschul et al., J. Mol. Biol., 215(3): 403-410 (1990); Altschul et al., Nature Genetics, 3:266-272 (1993)).

Protein, polypeptide, and peptide homologues within the scope of the present invention will be about 70%, preferably about 80%, and more preferably about 90% or more (including about 95%, about 97%, or even about 99% or more) homologous to a Nercc1 as disclosed herein. Polynucleotide homologues within the scope of the present invention will be about 60%, preferably about 70%, more preferably about 80%, and even more preferably about 90% or more (including about 95%, about 97%, or even about 99% or more) homologous to the nucleotide sequences described herein that encode a Nercc1 kinase, as disclosed herein.

The meaning of other terms will be evident as they are used in the text.

Nercc1, Nek6, and Nek7 Kinases

A Nercc1 kinase useful in the invention is any molecule that provides functional Nercc1 kinase activity, including, but not limited to, wild type Nercc1 kinase protein (e.g., having the amino acid sequence), enzymatically active mutant variants of Nercc1 kinase, and enzymatically active fusion proteins comprising all or a portion of Nercc1 kinase, as described or exemplified herein.

Wild type Nercc1 kinase is a NIMA-like kinase (also called a Nek9) that specifically phosphorylates Nek6 and Nek7 in the stepwise cascade of phosphorylation steps that activate kinases to ultimately signal a cell to enter mitosis. Nercc1 is also capable of auto-activation (auto-phosphorylation) by incubation in the presence of a nucleoside triphosphate, such as ATP or GTP, under standard kinase reaction conditions, e.g., in the presence of magnesium or manganese cation, pH 7.0 buffer, 25-28° C.

A human cDNA sequence encoding a wild type Nercc1 linase protein has the nucleotide sequence of SEQ ID NO: 1 and the corresponding deduced 979 amino acid sequence of SEQ ID NO:2 (Nercc1 sequence data are also available at Genbank Accession No. AY080896). The nucleotide sequence includes 23 exons that span a region of approximately 45 kilobase pairs (kbp) on human chromosome 14 in a region mapping at 14q24.3.

A human Nek6 protein useful in the compositions and methods described herein is encoded by the nucleotide sequence of SEQ ID NO:3 and has the amino acid sequence of SEQ ID NO:4. A human Nek7 protein useful in the compositions and methods described herein is encoded by the nucleotide sequence of SEQ ID NO:5 and has the amino acid of SEQ ID NO:6.

In addition to wild type Nercc1, Nek6, and Nek7 proteins, a variety of fusion proteins and mutant variants of these wild type kinase proteins are useful in compositions and methods of the invention and may be readily prepared using the available nucleotide coding and corresponding amino acid sequences and standard methods known in the art (e.g., recombinant or synthetic methods). Accordingly, a nucleic acid molecule of the invention may also comprise all or a portion of the Nercc1, Nek6, or Nek7 coding sequences (SEQ ID NOS: 1, 3, 5, respectively) as well as nucleotide sequences encoding one or more other polypeptides.

The nucleic acid molecules described herein may be in the form deoxyribonucleic acid (DNA), including synthetic DNA and cDNA, or ribonucleic acid (RNA). The DNA molecules may be double-stranded or single-stranded, and if single-stranded, may be the coding strand or non-coding (anti-sense) strand. The coding sequence for a Nercc1, Nek6, or Nek7 polypeptide may be manipulated or varied in known ways to yield alternative coding sequences that, as a result of the redundancy or degeneracy of the genetic code, encodes the same polypeptide. DNA molecules comprising the nucleotide coding sequences of Nercc1, Nek6, and Nek7 proteins may also be altered at one or more nucleotides by standard methods to produce mutant variant forms and fusions proteins described herein. Thus, nucleotide coding sequences for Nercc1, Nek6, and Nek7 proteins as described herein or elsewhere provides the means to readily produce a wild type form of these proteins or mutant variants thereof that are useful in the methods and compositions of the invention, e.g., by using recombinant DNA techniques, site-directed mutagenesis, and/or polymerase chain reaction (PCR) methods available in the art. In addition, such methods may employ any of a variety of nucleic acid vector molecules known in the art to express a recombinant protein described herein. Nucleic acid vector molecules useful in the methods of the invention include, but are not limited to, plasmids, bacteriophage vectors, mammalian viral vectors, baculoviral vectors, mini-chromosomes, transgenic vectors, and combinations thereof.

The full-length Nercc1 protein kinase encoded by a cloned cDNA is 979 amino acids in length (see, SEQ ID NO:2) and comprises several well-defined domains as indicated diagrammatically in FIG. 1 and listed below:

a Nercc1 kinase catalytic domain at amino acids 52-309 of SEQ ID NO:2 (“Protein Kinase” in FIG. 1), which is required for Nercc-mediated phosphorylation of a substrate;

a nuclear localization signal (“NLS” in FIG. 1) comprising two classical localization motifs: amino acid sequence PLLRKRRR at amino acids 306-313 of SEQ ID NO:2 and amino acid sequence PTKRPR at amino acids 325-330 of SEQ ID NO:2;

an RCC1 auto-inhibitory domain at amino acids 347-726 of SEQ ID NO:2 (“RCC1” in FIG. 1);

a glycine-rich region at amino acids 752-760 of SEQ ID NO:2 (“Gly” in FIG. 1);

possible SH3 domain binding motifs containing proline rich region (“PXXP” region) at amino acids 823-830 and 881-888 of SEQ ID NO:2 (see, “PXXP” region in FIG. 1); and

a “coiled coil” domain at amino acids 891-940 of SEQ ID NO:2 (see, “Coiled-coil (Homodimerization)” in FIG. 1), used for forming homodimers and possibly larger oligomeric forms of Nercc1 kinase.

The RCC1 domain functions as an auto-inhibitory domain of Nercc1 kinase. In the presence of nucleoside triphosphate, such as ATP, and divalent metal cation, such as magnesium or manganese cations, molecules of Nercc1 kinase can self-activate by auto-phosphorylation. While not wishing to be limited to any particular theory or mechanism, it appears that Nercc monomeric protein molecules associate as dimers or higher multimers by non-covalent association between “coiled coil” domains of monomers such that, in the presence of nucleoside triphosphate and standard kinase reaction conditions, one monomer of a multimer is able to phosphorylate another associated monomer in the multimer. Mutant variants of wild type Nercc are described herein that lack the RCC1 auto-inhibitory domain. Such RCC1-deletion variants are permanently (constitutively) activated, fully functional Nercc kinases.

The coiled coil region is necessary for Nercc to be active. Accordingly, it appears that Nercc must at least be in a homodimeric form for functional kinase activity.

The compositions and methods of the invention comprising Nercc1 kinase, Nek6, and/or Nek7 proteins are not limited to using a purified wild type or recombinantly expressed form of such proteins. A variety of Nercc1 kinase, Nek6, and Nek7 proteins are described herein that may find use in the invention. For example, as noted above, a particularly useful mutant variant of wild type Nercc1 kinase is a deletion mutant protein lacking the RCC1 auto-inhibitory domain, e.g., the mutant variant “Nercc (A347-308)” kinase protein. Such RCC1-deficient mutant variants are permanently activated and provide an otherwise fully functional Nercc1 kinase activity. Accordingly, an RCC1-deletion variant of wild type Nercc1 requires no preincubation with nucleoside triphosphate for activation of the Nercc1 kinase activity. Also useful in the invention are fusion proteins comprising a functional Nercc1 kinase linked to another protein or peptide that provides a convenient means to isolate or detect the Nercc fusion protein in detection assays. Examples of such Nercc fusion proteins include, but are not limited to, glutathione S-transferase (GST) fusions and epitope tag fusion proteins. Particularly preferred epitope tags for preparing fusion proteins for the invention include, without limitation, such well known tags as FLAG, HA (hemaglutinin), myc, and combinations thereof. With the availability of DNA molecules comprising the nucleotide coding sequence for Nercc, Nek6, and Nek7, fusion proteins comprising a Nercc1, Nek6, and/or Nek7 kinase protein or mutant variant linked to a GST protein or an epitope tag peptide are readily prepared by standard recombinant methods known in the art or prepared using commercially available kits.

Methods of Identifying Inhibitors of Mitosis Affecting NIMA-like Kinase Activity

The invention provides compositions and methods that may be used to identify test compounds that inhibit mitosis (i.e., anti-mitotic compounds) based on the ability to inhibit activity of one or more of the NIMA-like kinases described herein (i.e., Nercc1 kinase, Nek6, and Nek7) that are involved in mitosis. Since these kinases are critical for entry into and maintenance of normal eukaryotic cell mitosis, a compound that inhibits the activity of one of these kinases is expected to inhibit unregulated or otherwise undesirable mitotic progression by eukaryotic cells, e.g., various cancer cells and/or eukaryotic microbial pathogens.

According to the invention, an example of a method of identifying a compound that is an inhibitor of mitosis may comprise the steps of:
(a) providing a kinase reaction mixture comprising a purine nucleoside triphosphate, a Nercc1 kinase protein, and a kinase substrate,
(b) incubating said kinase reaction mixture in the presence and absence of a test compound for a time sufficient to permit the Nercc1 kinase protein to phosphorylate the kinase substrate, and
(c) detecting the level of phosphorylated kinase substrate produced in the presence and absence of the test compound,
   wherein a lower level of phosphorylated kinase substrate produced in the presence of the test compound compared to the level produced in the absence of the test compound indicates that the test compound is an inhibitor of mitosis.

Another example of a method of the invention for identifying an inhibitor of mitosis may comprise:
(a) providing a kinase reaction mixture comprising an activated Nek6 or Nek7 kinase protein, a kinase substrate, and a purine nucleoside triphosphate,
(b) incubating said reaction mixture in the presence and absence of a test compound for a time sufficient to permit the activated Nek6 or Nek7 kinase protein to phosphorylate said kinase substrate, and
(c) detecting the level of phosphorylated kinase substrate in the presence and absence of said test,
   wherein a lower level of phosphorylated kinase substrate produced in the presence of the test compound compared to the level produced in the absence of the test compound indicates that the test compound is an inhibitor of mitosis.

Any of a variety of Nercc1 kinase proteins (i.e., full-length Nercc or mutant derivatives thereof) described herein may be used in methods of the invention. All Nercc1 kinase proteins useful in methods of the invention must be or become activated, e.g., by auto-phosphorylation, or be permantly activated, e.g., as in the above-mentioned mutant variant Nercc (Δ347-732) protein that is deleted in the Nercc RCC1 auto-inhibitory domain.

Nek6 and Nek7 are in vivo substrates of Nercc1 kinase (see, e.g., Example 12, below). However, activated Nercc1 kinase, Nek6, and Nek7 proteins will transfer a phosphate group from a donor molecule (e.g., a nucleoside triphosphate) under in vitro kinase reaction conditions to any of a variety of acceptor molecules. A number of such in vitro kinase substrates are known in the art and include, but are not limited to, histone proteins (e.g., histone H3, histone H4), casein, Cdc16, p70S6K, and myelin basic protein (MBP). Cdc16 is particularly preferred as a substrate kinase for Nek6 and Nek7. Kinase substrates useful in the invention may be readily prepared by standard methods, including standard protein purification schemes and/or recombinant DNA technology, or may be obtained commercially in purity and amounts that permit the in vitro screening methods of the invention to be run in a routine and scaled-up format.

Non-activated (non-phosphorylated) Nercc1 kinase is capable of auto-activation by auto-phosphorylation as readily observed in standard in vitro kinase reaction mixtures comprising purine nucleoside triphosphate. Thus, in some embodiments of the invention, a non-activated Nercc1 kinase protein may not only be used as a self-activating NIMA-like kinase that provides kinase activity, but may also serve as the kinase substrate. In such embodiments, the level of auto-phosphorylated Nercc1 kinase protein may be determined in the presence and absence of a particular test compound. Alternatively, the level of auto-activation of the Nercc1 kinase protein may be detected or quantified as enzyme activity in a standard kinase assay.

Methods and compositions of the invention preferably use either ATP or GTP as the source of a phosphate group that is transferred in a kinase reaction. Since most kinases known in the art are active only with ATP, the use of GTP in the methods of the invention provides an additional degree of specificity to the method for identifying compounds that specifically inhibit mitosis. As with other kinase reactions, optimal activities are obtained using a metal cation, such as divalent magnesium or manganese cation, as counter ion to the nucleoside triphosphate. Magnesium divalent cation is particularly preferred for use in the methods and compositions of the invention.

Any buffer used in standard kinase reactions (phosphorylations) may be used in the kinase reaction of the screening methods of the invention. Preferably, the pH of the buffer is between pH 7.0-7.5. A particularly preferred buffer is a phosphorylation buffer comprising 50 mM MOPS (3-[N-morpholino]propanesulfonic acid), pH 7.4; 1 mM dithiothreitol (DTT); 1 mM EGTA (calcium chelating agent); 5 mM MgCl₂; 10 mM β-glycerophosphate; and 25 nM calyculin (see, Examples section).

According to the invention, the level of inhibition of mitosis is correlated to the level of inhibition of NIMA-like kinase activity as determined by the amount of phosphorylated substrate (kinase reaction product). Phosphorylated kinase substrates from a NIMA-like kinase reaction may be detected and measured using any of a variety of methods known in the art for detecting and measuring phosphorylated proteins. For example, a NIMA-like kinase reaction may transfer a detectably labeled phosphate group from a donor molecule to a kinase substrate. Such detectable labels include radioactive isotopes, such as ³²P transferred from γ-³²P-labeled nucleoside triphosphate. Radioactively labeled phosphorylated substrate products are readily detected and measured, e.g., by autoradiography, scintillation counter, Cerenkov counting, and other methods of detecting radiolabeled molecules. Non-radioactive labels for transferred phosphate groups may include biotin, which can form a complex with an avidin or streptavidin molecule. A variety of detection systems based on a biotinylated product and an avidin- or streptavidin-conjugated detection protein or enzyme are commercially available in the art.

Alternatively to detecting a transferred phosphate group, kinase activity may be determined using an antibody that is specific for the phosphorylated substrate. Examples of such phosphospecific antibodies are described herein (see, Examples section, below) in which an antibody is employed that specifically binds to the phosphorylated form of a kinase substrate. For example, Nek6 or Nek7 kinase proteins may be phosphorylated by Nercc1 kinase at specific phosphorylation sites (P sites) that are critical for activation of these Nek kinase proteins (see, e.g., Examples 1 and 12, below). Such P sites that are critical to activation of Nek6 are serine206 and, to a lesser extent, threonine202 in the Nek6 amino acid sequence (SEQ ID NO:4). In Nek7, a critical phosphorylation site for activation is serine195 (corresponding to serine206 in Nek6) in the Nek7 amino acid sequence (SEQ ID NO:6). The availability of such phosphospecific antibodies advantageously permits the use of any of a variety of immuno-detection methods and formats that are available in the art or described herein to screen for test compounds that inhibit mitosis.

Compounds initially identified as inhibitors of mitosis (i.e., anti-mitotic compounds) according to a method described above may be further characterized for the ability to inhibit or halt mitosis in proliferating (actively dividing) cells, either in vitro or in vivo. The cells employed in this further characterization step may be any of a variety of proliferating cells, including but not limited to, non-cancerous cells, cancer cells, or cells of a eukaryotic pathogen of interest. Preferably, the proliferating cells are cancer cells or cells of a eukaryotic pathogen of interest. The cells may be proliferating in vitro, e.g., in cell cultures, or in vivo, e.g., in animal models, such as an animal model for a cancer or infectious disease of interest. A preferred method for this further characterization of an inhibitor of mitosis is to contact actively dividing cells in culture with previously identified anti-mitotic compound, and then assay for disruption of microtubules (mitotic spindles), misalignment of chromosomes, inhibition of proliferation, cell lysis, or apoptosis (i.e., programmed cell death). Particularly preferred is to determine whether a compound causes a disruption of the mitotic spindles and/or misalignment of chromosomes. For example, such disruptions in the cellular process of mitosis are documented effects of specifically inhibiting Nercc1 kinase activity (see, e.g., FIGS. 6A and 6B) and, therefore, the involvement of the cascade comprising Nercc1 and Nek6/7 proteins in mediating mitosis as described herein. Accordingly, such a further characterization step in methods of the invention may indicate the particular stage of mitosis that is disrupted by a particular anti-mitotic compound. A disruption of the mitotic spindles (mitotic microtubules) and/or of the alignment of chromosomes in cells undergoing mitotic progression are readily revealed by standard staining methods for microtubules (e.g., using anti-tubulin antibody that binds spindles and a secondary antibody conjugated to a dye, such as rhodamine) and for DNA (e.g., using any well-known dye for DNA visualization, such as 4,6-diamidino-2-phenylindole (“DAPI”, Sigma, St. Louis, Mo.); HOECHST 33342 (Hoechst)) in cells. Such methods may be conveniently employed and analyzed in small cultures, such as wells of a microtiter culture plate.

The invention also provides methods of diagnosing a cancerous or potentially cancerous (including pre-cancerous) condition in cells or tissues of an individual based on detecting a notable or unusual elevation in the amount of Nercc1, Nek6, and/or Nek7 kinase protein produced or in the level of one or more of the corresponding kinase activities in cells or tissue from the individual. A change in the level of kinase protein or kinase activity may be detected by comparing the level of linase protein or kinase activity in a particular sample of cells from an individual with the level found or expected in normal or healthy cells or tissue (i.e., as a standard). Alternatively, a change may be detected by comparing levels of kinase protein or kinase activity over time in multiple samples of cells from an individual. Levels of kinase protein expression or kinase activities in a sample of cells may be determined using any of a variety of assays including, but not limited to, a kinase-mediated phosphate transfer reaction (e.g., carried out in a reaction vessel or an in-gel assay); an immunodetection assay for Nercc1, Nek6, or Nek7 (e.g., using an antibody to detect Nercc1, Nek6, Nek7, or a phosphorylated form thereof); and determination of levels of mRNA transcripts encoding Nercc1, Nek6, or Nek7 (e.g., Northern analysis, quantitative PCR). Cells of an individual for such diagnostic analysis as described herein may be obtained from any of a variety of sources, including but not limited to, tissue biopsies, blood, cell smears, tissue swabs, bodily fluids, feces, and other samples routinely obtained for cancer screening.

Other variations and embodiments of this invention described herein will now be apparent to those of ordinary skill in the art without departing from the scope of this invention.

### EXAMPLES

### Example 1

### Protein Sequencing, cDNA Cloning, Manipulations, Assays, and Nomenclature for Derivatives of Nercc1, Nek6, and Nek7 Kinase Proteins

Sequence analysis of a tryptic digest of Nercc1 kinase (p120 protein) was performed at the Harvard Microchemistry Facility at Harvard University, Cambridge, Mass., by microcapillary reverse-phase HPLC nano-electrospray tandem mass spectrometry (μLC/MS/MS) on a Finnigan LCQ quadrupole ion trap mass spectrometer. Online, data-dependent scans enabled a determination of charge state and exact mass, followed by the acquisition of tandem mass spectra. The identification of spectra corresponding to known peptide sequences in the NCBI databases was enabled with the algorithm Sequest, followed by manual confirmation.

A cDNA encoding Nercc polypeptide fragments (amino acids 1-308 and amino acids 1-391 of SEQ ID NO:2) was obtained by polymerase chain reaction (PCR) using the EST AI961740 as template and primers having the following sequences, each containing a BglII site:

for sequence encoding Nercc fragment consisting of amino acids 1-308 of SEQ ID NO:2:

| | | |
|---|---|---|
| sense- | 5′ GAAGATCTACGCCGCCATGTCGG | (SEQ ID NO:7) |
| | TGCTGGG 3′, | |
| antisense- | 5′ GAAGATCTCTAGAGAAGAGGGCG | (SEQ ID NO:8) |
| | ATCTAGAAG 3′; | |

for sequence encoding Nercc fragment consisting of amino acid 1-391 of SEQ ID NO:2:

| | | |
|---|---|---|
| sense- | 5′ GAAGATCTACGCCGCCATGTC | (SEQ ID NO:9) |
| | GGTGCTGGG 3′, | |
| antisense- | 5′GTACAGTTCCTTCTCCAC | (SEQ ID NO:10) |
| | TG 3′. | |

The cDNA encoding a Nercc fragment consisting of amino acids 1-391 of SEQ ID NO:2 was inserted into a T-vector (PCR 2.1., Invitrogen) and excised with BglII and HindIII, so as to include a stop codon from the T-vector. A full-length Nercc cDNA was obtained by PCR, using as template the “Nercc (1-391)”-encoding cDNA (above) and a PCR fragment amplified from a human skeletal muscle library (Clontech) with primers having the following sequences:

| | | |
|---|---|---|
| sense- | 5′CAGTGGAGAAGGAACTGT | (SEQ ID NO:11) |
| | AC3′, | |
| antisense | 5′CTGTGTCTCCTCTTCAAA | (SEQ ID NO:12) |
| | GG3′. | |

A cDNA encoding Nercc fragments encoding amino acids 338-739 or amino acids 338-979 of SEQ ID NO:2 were amplified using the following primers containing XbaI sites and having the following sequences:

for cDNA sequence encoding Nercc fragment consisting of amino acids 338-739 of SEQ ID NO:2:

| | | |
|---|---|---|
| sense- | 5′CGGCTCTAGACATTGCTGTAGT | (SEQ ID NO: 13) |
| | AACATCACG3′, | |
| antisense- | 5′CGGCTCTAGACTTAGCCACTGC | (SEQ ID NO: 14) |
| | TATTGGAACGGATGG3′. | |

for cDNA sequence encoding Nercc fragment consisting of amino acids 338-979 of SEQ ID NO:2:

| | | |
|---|---|---|
| sense- | 5′CGGCTCTAGACATTGCTGTAGT | (SEQ ID NO: 15) |
| | AACATCACG3′, | |
| antisense- | 5′CGGCTCTAGAGGGGCTCTATAG | (SEQ ID NO: 16) |
| | GCTCAGG3′. | |

All of the Nercc-encoding cDNA molecules described above were inserted into pCMV5 FLAG vector (Andersson et al., J. Biol. Chem., 264: 8222-8229 (1989)) and verified by DNA sequence determination.

The cDNA encoding Nercc fragment consisting of amino acids 732-979 of SEQ ID NO:2 was amplified from a human skeletal muscle cDNA library using primers, which contain NotI and SpeI sites, respectively, having the following sequences:

| | | |
|---|---|---|
| sense- | AAATATGCGGCCGCAACCATCC | (SEQ ID NO:17) |
| | GTTCCAATAGCAGTGG3′, | |
| antisense- | 5′CCTGATCAGGGCTCTATAGGGT | (SEQ ID NO:18) |
| | CAGGAG3′. | |

After digestion with Not1/Spe1, this cDNA was inserted into the vector pEBG for expression as a GST-fusion.

All other Nercc coding variants were constructed using standard PCR mutation techniques or the QuikChange Site-Directed Mutagenesis kit (Stratagene, La Jolla, Calif.).

Nomenclature for Mutatnt Variant and Fusion Proteins

A variety of proteins were constructed from the full-length Nercc having amino acid sequence of SEQ ID NO:2 encoded by nucleotides 1-2937 of SEQ ID NO: 1, the full-length Nek6 amino acid sequence of SEQ ID NO:4 encoded by the nucleotide sequence of SEQ ID NO:3, or the full-length Nek7 amino acid sequence of SEQ ID NO:6 encoded by nucleotides of SEQ ID NO:5 for use in the studies described herein. A standard nomenclature was used to designate the various proteins employed in the studies described herein.

In the case of mutant variant forms of Nercc1, Nek6, and Nek7, the amino acids of the full-length kinase amino acid sequence are indicated following the term “Nercc”, “Nek6”, and “Nek7”. For example, the terms “Nercc (1-391)” or, simply, “Nercc 1-391” designates a protein having an amino acid sequence consisting of amino acids 1-391 of SEQ ID NO:2 of the full-length Nercc protein. In the case of mutant forms of Nercc protein having an amino acid residue change, the term “Nercc” is followed by an abbreviation indicating the amino acid residue that has been changed by mutation, e.g., “Nercc (K81M)” or “Nercc K81M” indicates that the lysine residue (K) at amino acid position 81 in the wild type Nercc amino acid sequence (SEQ ID NO:2) has been replaced by mutation with a methionine residue (M). The designation “Nercc (1-391, K81M)” indicates a deletion mutant (truncated) form of Nercc, which consists of the amino terminal 391 amino acids, i.e., amino acids 1-391 of the full-length, wild type Nercc amino acid sequence (SEQ ID NO:2), in which the lysine residue at amino acid position 81 of wild type Nercc in SEQ ID NO:2 was replaced by mutation with a methionine residue. “Nercc (Δ763-889)” indicates a Nercc mutant variant protein in which amino acids 763-889 of SEQ ID NO:2 have been deleted from the full-length, wild type Nercc 979-amino acid sequence of SEQ ID NO:2.

Fusion proteins comprising a kinase or portion thereof, which is fused to another, non-Nercc/Nek kinase, are designated using an abbreviation for the non-kinase protein followed by the designation for the particular kinase protein. For example, “GST Nercc” refers to a fusion protein comprising a glutathione S-transferase protein (GST) linked in frame to a full-length Nercc protein. GST is encoded by the nucleotide sequence of SEQ ID NO:19 and has the amino acid sequence of SEQ ID NO:20.

A number of fusion proteins are designated with an appended name of one or more epitope tag polypeptides fused to a kinase protein or portion thereof:

The FLAG epitope tag polypeptide is encoded by the nucleotide sequence of SEQ ID NO:21 and has the amino acid sequence of SEQ ID NO:22;

The HA (hemagglutinin) epitope tag polypeptide is encoded by the nucleotide sequence of SEQ ID NO:23 and has the amino acid sequence of SEQ ID NO:24;

The myc epitope tag polypeptide is encoded by the nucleotide sequence of SEQ ID NO:25 and has the amino acid sequence of SEQ ID NO:26.

The above nomenclature is demonstrated in the following examples:

“Nercc (K81M)” or “Nercc K81M”, a polypeptide having the amino acid sequence of SEQ ID NO:2, except that the lysine at position 81 has been replaced with a methionine by mutating lysine codon nucleotides AAG at 241-243 of SEQ ID NO:1 to ATG. “Nercc (1-308)” or “Nercc 1-308”, a polypeptide comprising the Nercc catalytic domain and having the sequence of amino acids 1-308 of SEQ ID NO:2 (encoded by the sequence of nucleotides 1-924 of SEQ ID NO: 1).

Nercc (1-391), a polypeptide having the sequence of amino acids 1-391 of SEQ ID NO:2 (encoded by the sequence of nucleotides 1-1173 of SEQ ID NO:1).

Nercc (1-391, K81M), a polypeptide having an amino acid sequence of amino acids 1-391 of SEQ ID NO:2, except that the lysine at position 81 has been replaced with methionine by mutating lysine codon nucleotides AAG at 241-243 of SEQ ID NO:1 to ATG.

Nercc (1-739), a polypeptide having the sequence of amino acids 1-739 of SEQ ID NO:2 (encoded by the sequence of nucleotides 1-2217 of SEQ ID NO: 1).

Nercc (1-891), a polypeptide having the sequence of amino acids 1-891 of SEQ ID NO:2 (encoded by the sequence of nucleotides 1-2673 of SEQ ID NO: 1).

Nercc (Δ347-732), a polypeptide having the amino acid sequence of amino acids 1-346,and 733-979 of SEQ ID NO:2 (encoded by the sequences of nucleotides 1-1038 and 2197-2937 of SEQ ID NO:1, respectively) and that possesses a permanently activated kinase activity without phosphorylation.

Nercc (Δ763-889), a polypeptide having the amino acid sequence of amino acids 1-762 and 890-979 of SEQ ID NO:2 (encoded by the sequences of nucleotides 1-2286 and 2668-2937 of SEQ ID NO:1, respectively).

Nercc (338-739), a polypeptide comprising the Nercc RCC1 domain and having the amino acid sequence of amino acids 338-739 of SEQ ID NO:2 (encoded by the sequence of nucleotides 1012-2217 of SEQ ID NO:1).

Nercc (338-979), a polypeptide having the amino acid sequence of amino acids 338-979 of SEQ ID NO:2 (encoded by the sequence of nucleotides of 1012-2937 of SEQ ID NO:1).

Nercc (732-979), a polypeptide having the amino acid sequence of amino acids 732-979 of SEQ ID NO:2 (encoded by the sequence of nucleotides 2194-2937 of SEQ ID NO: 1).

Nercc (891-940), a polypeptide having amino acids 891-940 of SEQ ID NO:2 (encoded by the sequence of nucleotides 2671-2820 of SEQ ID NO: 1).

FLAG Nercc is a fusion protein comprising the FLAG epitope tag polypeptide having the amino acid sequence of SEQ ID NO:22 linked in frame to Nercc1 having the amino terminus of SEQ ID NO:2.

FLAG Nercc (1-891) is a fusion protein comprising the FLAG polypeptide linked in frame to the amino terminus of a Nercc polypeptide having the amino acid sequence of amino acids 1-891 of SEQ ID NO:2.

FLAG Nercc (Δ347-732) is a fusion protein comprising the FLAG polypeptide linked in frame to the amino terminus of a Nercc polypeptide having the amino acid sequence of amino acids 1-346 and 733-979 of SEQ ID NO:2.

FLAG Nercc (Δ347-732) (K81M) is a fusion protein comprising the FLAG polypeptide (SEQ ID NO:22) linked in frame to the amino terminus of a Nercc polypeptide having the amino acid sequence of amino acids 1-346 and 733-979 of SEQ ID NO:2, except that the lysine at position 81 of SEQ ID NO:2 has been replaced with methionine by mutating lysine codon AAG at nucleotides 241-243 of SEQ ID NO:1 to ATG.

FLAG Nercc (338-979) is a fusion protein comprising the FLAG polypeptide (SEQ ID NO:22) linked in frame to the amino terminus of a Nercc polypeptide having an amino acid sequence of amino acids 338-979 of SEQ ID NO:2.

HA Nercc (1-391) is a fusion protein comprising the HA epitope tag polypeptide having the amino acid sequence of HA (SEQ ID NO:24) linked in frame to the amino terminus of a Nercc polypeptide having an amino acid sequence of amino acids 1-391 of SEQ ID NO:2.

GST Nercc (891-940) is a fusion protein comprising the GST protein having the amino acid sequence of GST (SEQ ID NO:20) linked in frame to the amino terminus of a Nercc polypeptide having an amino acid sequence of amino acids 891-940 of SEQ ID NO:2.

GST Nercc (732-979) is a fusion protein comprising the GST protein having the amino acid sequence of GST (SEQ ID NO:20) linked in frame to the amino terminus of a Nercc polypeptide having an amino acid sequence of amino acids 732-979 of SEQ ID NO:2.

FLAG Nek6 is a fusion protein comprising the FLAG polypeptide having the amino acid sequence of SEQ ID NO:22 linked in frame to the amino terminus of Nek6 having an amino acid sequence of SEQ ID NO:4.

myc Nek6 is a fusion protein comprising the myc epitope tag polypeptide having the amino acid sequence of myc (SEQ ID NO:26) linked in frame to the amino terminus of Nek6 (SEQ ID NO:4).

Nek6 S37A is a polypeptide having the amino acid sequence of SEQ ID NO:4, except that the serine at position 37 of SEQ ID NO:4 has been replaced with an alanine by mutating the serine codon TCT at nucleotides 109-111 of SEQ ID NO:3 to GCT.

Nek6 S131A is a polypeptide having the amino acid sequence of SEQ ID NO:4, except that the serine at position 131 of SEQ ID NO:4 has been replaced with an alanine by mutating the serine codon nucleotides TCG at nucleotides 391-393 of SEQ ID NO:3 to GCG.

Nek6 S206A is a polypeptide having the amino acid sequence of SEQ ID NO:4, except that the serine at position 206 of SEQ ID NO:4 has been replaced with an alanine by mutating the serine codon TCC at nucleotides 616-618 of SEQ ID NO:3 to GCC.

Nek6 S206D is a polypeptide having the amino acid sequence of SEQ ID NO:4, except that the serine at position 206 of SEQ ID NO:4 has been replaced with an aspartic acid by mutating the serine codon TCC at nucleotides 616-618 of SEQ ID NO:3 to GAT.

Nek6 S37D is a polypeptide having the amino acid sequence of SEQ ID NO:4, except that the serine at position 37 of SEQ ID NO:4 has been replaced with an aspartic acid by mutating the serine codon TCT at nucleotides 109-111 of SEQ ID NO:3 to GAT.

Nek6 T202E is a polypeptide having the amino acid sequence of SEQ ID NO:4, except that the threonine at position 202 of SEQ ID NO:4 has been replaced with a glutamic acid by mutating the threonine codon ACC at nucleotides 604-606 of SEQ ID NO:3 to GAA.

Nek6 T202A is a polypeptide having the amino acid sequence of SEQ ID NO:4, except that the threonine at position 202 of SEQ ID NO:4 has been replaced with an alanine by mutating threonine codon ACC at nucleotides 604-606 of SEQ ID NO:3 to GCC.

Nek6 T202C is a polypeptide having the amino acid sequence of SEQ ID NO:4, except that the threonine at position 202 of SEQ ID NO:4 has been replaced with a cysteine by mutating the threonine codon ACC at nucleotides 604-606 of SEQ ID NO:3 to TGC.

Nek6 S198A/S199A is a polypeptide having the amino acid sequence of SEQ ID NO:4, except that the serine at position 198 of SEQ ID NO:4 has been replaced with an alanine by mutating the serine codon AGC at nucleotides 592-594 of SEQ ID NO:3 to GCC, and the serine at position 199 of SEQ ID NO:4 has been replaced with an alanine by mutating the serine codon TCT at nucleotides 595-597 of SEQ ID NO:3 to GCT.

Nek6 T201A/T202A is a polypeptide having the amino acid sequence of SEQ ID NO:4, except that the threonine at position 201 of SEQ ID NO:4 has been replaced with an alanine by mutating the threonine codon ACC at nucleotides 601-603 of SEQ ID NO:3 to GCC, and the threonine at position 202 of SEQ ID NO:4 has been replaced with an alanine by mutating the threonine codon ACC at nucleotides 604-606 of SEQ ID NO:3 to GCC.

Nek6 T201A/T202A/S206A is a polypeptide having the amino acid sequence of SEQ ID NO:4, except that the threonine at position 201 of SEQ ID NO:4 has been replaced with an alanine by mutating the threonine codon ACC at nucleotides 601-603 of SEQ ID NO:3 to GCC, the threonine at position 202 of SEQ ID NO:4 has been replaced with an alanine by mutating the threonine codon ACC at nucleotides 604-606 of SEQ ID NO:3 to GCC, and the serine at position 206 of SEQ ID NO:4 has been replaced with an alanine by mutating the serine codon TCC at nucleotides 616-618 of SEQ ID NO:3 to GCC.

Nek6 T201A/T202A/S206D is a polypeptide having the amino acid sequence of SEQ ID NO:4, except that the threonine at position 201 of SEQ ID NO:4 has been replaced with an alanine by mutating the threonine codon ACC at nucleotides 601-603 of SEQ ID NO:3 to GCC, the threonine at position 202 of SEQ ID NO:4 has been replaced with an alanine by mutating the threonine codon ACC at nucleotides 604-606 of SEQ ID NO:3 to GCC, and the serine at position 206 has been replaced with an aspartic acid by mutating the serine codon TCC at nucleotides 616-618 of SEQ ID NO:3 to GAC.

Nek6 T202E/S206A is a polypeptide having the amino acid sequence of SEQ ID NO:4, except that the threonine at position 202 of SEQ ID NO:4 has been replaced with a glutamic acid by mutating the threonine codon ACC at nucleotides 604-606 of SEQ ID NO:3 to GAA, and the serine at position 206 of SEQ ID NO:4 has been replaced with an alanine by mutating the serine codon TCC at nucleotides 616-618 of SEQ ID NO:3 to GCC.

Nek6 T202E/S206D is a polypeptide having the amino acid sequence of SEQ ID NO:4, except that the threonine at position 202 of SEQ ID NO:4 has been replaced with a glutamic acid by mutating the threonine codon ACC at nucleotides 604-606 of SEQ ID NO:3 to GAA, and the serine at position 206 of SEQ ID NO:4 has been replaced with an aspartic acid by mutating the serine codon TCC at nucleotides 616-618 of SEQ ID NO:3 to GAC.

Nek6 K74M/K75M is a polypeptide having the amino acid sequence of SEQ ID NO:4, except that the lysine at position 74 of SEQ ID NO:4 has been replaced with a methionine by mutating the lysine codon AAG at nucleotides 220-222 of SEQ ID NO:3 to ATG, and the lysine at position 75 of Nek6 has been replaced with a methionine by mutating the lysine codon AAG at nucleotides 223-225 AAG of SEQ ID NO:3 to ATG.

Designations for other proteins described herein will be evident by analogy with the above system of nomenclature.

The construction of plasmids pCMV5 FLAG-Nek6 and pEBG-Nek6 has been described elsewhere (Belham et al., Curr. Biol., 11: 1155-1167 (2001)). GFP-Nercc fusion proteins were generated by subcloning nucleic acid comprising nucleotide sequences encoding Nercc wild type, Nercc (K81M), and Nercc (1-391) into pEGFP-C2 vector (Clontech).

For prokaryotic expression, cDNAs were inserted into the pGEX vector (Pharmacia Biotech), expressed as GST-fusion proteins and purified on GSH-agarose.

Northern Analysis

Poly-A mRNA was purified from mouse tissues; after electrophoresis and blot transfer, the blot was sequentially hybridized with a ³²P-labeled EcoRI restriction fragment of Nercc followed by a beta-actin cDNA, using standard conditions.

Cell Culture and Transfection

HEK293 cells were maintained in Dulbecco's modified Eagle's medium (DMEM, GIBCO brand, invitrogen, Carlsbad, Calif.) whereas CF-PAC-1 cells were maintained in Iscove's modified DME, both supplemented with 2 mM L-Glutamine and 100 U/ml penicillin-streptomycin (GIBCO brand, Invitrogen, Carlsbad, Calif.), plus 10% fetal calf serum (FCS, Sigma, St. Louis, Mo.). Cells were transfected with LIPOFECTAMINE (GIBCO brand, Invitrogen, Carlsbad, Calif.) transfecting reagent according to the manufacturer's instructions.

HeLa, CHO and BHK-21 cells were maintained in DMEM plus 10% FCS, supplemented with 2 mM L-glutamine and 100 U/ml penicillin-streptomycin. For the growth of NIH 3T3 and Swiss 3T3 cells, 10% calf serum was used. PtK2 cells were grown in minimum essential medium (MEM, GIBCO brand, Invitrogen, Carlsbad, Calif.) containing 2 mM L-glutamine and Earle's salts supplemented with non-essential amino acids (Sigma, St. Louis, Mo.) and 10% fetal bovine serum.

Cell Lysis, Immunoprecipitation In Vitro Binding and Immunobloting

Cells were rinsed with PBS, flash-frozen in liquid nitrogen, and stored at −70° C. Cell lysis employed a buffer containing: 50 mM Tris pH 7.1, 100 mM NaCl, 1 mM DTT, 1 mM EDTA, 1 mM EGTA, 10 mM β-glycerophosphate, 2 mM Na₃VO₄, 25 nM calyculin A, 1% TX100, plus protease inhibitors (EDTA-free tablets, Roche). Protein concentration was determined by the Bradford reagent method (BioRad, Hercules, Calif.). Immunoprecipitations were carried out with the indicated antibodies prebound to protein A/G-agarose (Santa Cruz), and washed in the lysis buffer containing in 0.5 M LiCl.

Immunoblotting was carried out after separation of proteins by SDS-PAGE and transfer to PVDF membranes; blots were probed with the antibodies indicated and bound antibodies were detected by ECL chemiluminiscence (Amersham Pharmacia Biotech, Piscataway, N.J.).

Gel Filtration

293 cells were lysed in 1% TX100 lysis buffer, ultracentrifuged at 100,000× g for 40 min, and loaded to a precalibrated HiPrep 16/60 Sephacryl S-300 High Resolution column (Amersham Pharmacia Biotech, Piscataway, N.J.). Gel filtration was carried out in 50 mM Tris pH 7.5, 150 mM NaCl, 1 mM DTT buffer at 0.5 ml/min.

Protein Kinase Assays

Protein kinase assays were carried out after immunoprecipitation; recombinant Nercc was isolated using anti-FLAG antibodies prebound to protein A/G-agarose beads. Endogenous Nercc was immunopurified using Ni antibodies prebound to protein A/G-agarose beads. Complexes were washed sequentially with lysis buffer and phosphorylation buffer (50 mM MOPS, pH 7.4, 1 mM DTT, 1 mM EGTA, 5 mM MgCl₂, 10 mM β-glycerophosphate, 25 nM calyculin A). Nercc auto-activation was carried out by incubation of immobilized Nercc in phosphorylation buffer plus 100 μM ATP at 25° C. for the indicated times. Activation was terminated by washing the immobilized Nercc in phosphorylation buffer, and the protein activity achieved was assayed by incubation at 30° C. in phosphorylation buffer supplemented with either 10 μM or 100 μM [γ-³²P]ATP, with an exogenous substrate, usually Histone H3, as indicated. Assays were stopped by addition of electrophoresis sample buffer and boiling, and the proteins were resolved by SDS-PAGE. ³²P incorporation was measured with a PhosphorImager imaging system or by liquid scintillation counting, as indicated.

Phosphoaminoacid Analysis

³²P-labeled histone H3 or Nercc polypeptides were excised from gels, subjected to 6 N HCl at 110° C. for 1 hour and brought to dryness. Aliquots were dissolved in pH 3.5 buffer (5% acetic acid, 0.5% pyridine) mixed with phosphoaminoacid standards, and separated by thin layer electrophoresis at pH 3.5.

Anti-phosphorylated Nek7 Antibody

Polyclonal antibody was raised in rabbits by injection of a peptide conjugated to keyhole limpet hemocyanin (KLH) by standard methods. The peptide conjugated to KLH had the following amino acid sequence:

CAAHS*LVGTPYYM (SEQ ID NO:27), wherein S* denotes a phosphoserine (corresponding to Ser195 of mouse Nek7).

Antibodies were purified by affinity chromatography using the immunizing peptide as affinity ligand. The resulting antibody bound phosphorylated Nek7, but not unphosphorylated Nek7.

Immunocytochemistry

Cells grown on coverslips were rinsed with PBS, fixed in methanol at −20° C. for 15 min, rinsed twice with PBS and incubated for 30 min at room temperature with the appropriate dilution of primary antibody in PBS. To visualize endogenous Nercc, affinity purified anti-Nercc peptide antibodies (N1 and C1) were employed at 10 μg/ml. Microtubules were visualized with a α/β-tubulin specific antibody. Coverslips were washed with PBS, and incubated with labeled secondary antibodies from corresponding species in appropriate combination-fluorescein or rhodamine X-conjugated donkey anti-rabbit, Cy2 or rhodamine X-conjugated donkey anti-mouse (each at 1:450). Incubation was terminated with a rinse in PBS, and the coverslips were mounted on a microscope slide. For immunodetection blocking, Nercc1 C1- or N1-antibody was incubated for 30 min at 37° C. with a 15-fold molar excess of immunizing peptide. After centrifugation at 12,000× g for 10 min, the mixture was used for immunoblotting or immunocytochemistry, as indicated. To examine the effects of Leptomycin B treatment on Nercc localization, HeLa or BHK21 cells were grown to subconfluency on 25 mm round glass coverslips in DMEM supplemented with 10% fetal bovine serum and penicillin-streptomycin. BHK21 cells were transfected (Fugene, Roche Molecular Biochemicals) with the wild type FLAG Nercc; medium was replaced after 18 hours (h). Six hours thereafter, Leptomycin B (2, 20, or 200 nM) was added; fixation in cold (−20° C.) methanol was carried out after 30 min. 60 min, 120 min, 180 min, 240 min, and 24 h. Nontransfected cells were stained for endogenous Nercc using affinity-purified anti-Nercc IgG followed by rhodamine labeled donkey anti-rabbit antibody. Transfected cells were stained with monoclonal anti-FLAG antibody followed by rhodamine-labeled donkey anti-mouse antibody.

Microinjections and Timelapse Recordings

For real time observation of the effects of recombinant Nercc expression, both wild type and variant, on cellular morphology and behavior during one cell cycle, HeLa cells grown on 25 mm glass coverslips were transfected with pEGFP-C2 vector or this vector encoding GFP fusions with Nercc wild type, Nercc (K81M), Nercc (1-391) using Fugene transfection reagent (Roche Molecular Biochemicals, Roche Diagnostics, Indianapolis, Ind.). Fugene-containing medium was removed after 12 hours, and DMEM supplemented with 10% calf serum and penicillin-streptomycin was added after rinsing. The percentage of transfected cells undergoing division within 24 hours was monitored. Using this transfection procedure, cells transfected with empty pEGFP-C2 underwent division at a frequency similar to nontransfected cells. The coverslips (in a Sykes-Moore chamber) were mounted on a microscope stage prewarmed to 37° C.; a region with the highest density of transfected cells (GFP-positive) was selected for observation, and phase contrast images were acquired using a 40×1.0 NA objective every 10 min for 25 hours. Light was kept to minimum during image acquisitions and shuttered between acquisitions.

To observe the effect of anti-Nercc antibodies on mitosis, PtK2 cells were grown to subconfluency on 25 mm round glass coverslips placed inside 35 mm cell culture dishes. A cell in prophase was found using phase contrast optics and microinjected in the period between the nucleolar disassembly and nuclear envelope breakdown either with 2.5 or 10 μg/μl normal rabbit IgG (Jackson Immunoresearch) for control experiments, or with 2.5 mg/ml rabbit anti-Nercc C- or N- terminus or kinase domain antibody with 0.5 μg/μl Rhodamine labeled dextran 3000 (Molecular Probes, Eugene, Oreg.). Typically, the volume of microinjected material comprised approximately 10% of cell volume. Immediately after microinjection, coverslip with microinjected cells was placed in a Sykes-Moore chamber (Bellco Glass, Vineland, N.J.) filled with bicarbonate-free DMEM supplemented with 10% fetal calf serum. The chamber was transferred onto the stage of a Zeiss Axiovert 100M microscope maintained at 37° C. with the aid of an Air-Therm heater controller (World Precision Instruments, Sarasota, Fla.), and a custom made microscope incubator. Microinjected cells were found by rhodamine fluorescence using a maximum possible density neutral density filter (typically, ND 1.0, Chroma Technology, Brattleboro, Vt.). Phase contrast images were acquired every 20 or 30 s with a Hamamatsu Orca-100 CCD camera driven by Metamorph 4.0 (Universal Imaging Corporation, Downingtown, Pa.); a 100×1.4 NA objective was used and light was kept to minimum during image acquisitions and shuttered between acquisitions.

### Example 2

### Analysis of Cloned cDNA Encoding Nercc1 Kinase

Immunoaffinity purification of a FLAG Nek6 polypeptide overexpressed in HEK293 cells results in the recovery of an associated 120 kilodalton (kDa) polypeptide (p120). Incubation of the FLAG Nek6 immunoprecipitate with Mg²⁺ plus [γ-³²P]ATP yields ³²P incorporation into both Nek6 and p120 to a similar extent, suggesting that p120 is a substrate for Nek6, a protein kinase itself, or both. The 120 kDa band was excised, subjected to tryptic digestion in situ, microcapillary reverse-phase HPLC and peptide sequence analysis by electrospray ionization mass spectrometry. Spectra corresponding to multiple peptide sequences were identified on each of three successive open reading frames (ORFs) predicted by GENESCAN (Burge et al., J. Mol. Biol., 268: 78-94 (1997)) on the human BAC clone 201F1 (AC007055). Although none of the predicted ORFs encode a polypeptide whose mass approaches 120 kDa, the sum of their molecular masses is close to this value, suggesting that the exon-intron boundaries had not been determined correctly by GENESCAN. Further analysis of the genomic sequences including and surrounding all three ORFs, using both GENEMARK (Borodovsky et al., Computers and Chem., 17: 123-133 (1993)) or GENESCAN yielded predictions containing all three original ORFs (AAD31938, AAD31939 and AAD31940) in polypeptides of around 100 kDa (107 kDa and 91 kDa, respectively).

Using the BLAST program, multiple overlapping human expressed sequence tags (ESTs) corresponding to the genomic sequences were identified, enabling the assembly of a continuous cDNA sequence (submitted to the NCBI GenBank database; Accession Number AY080896). The gene corresponding to this mRNA is encoded by 23 exons that span a region of about 45 kbp on human chromosome 14, in a region mapping at 14q24.3. The predicted sequence has been confirmed by EST sequencing (NCBI GenBank Accession Numbers A1961740, A1799812, AA 836348, and AA568111), as well as by DNA sequence obtained from a series of overlapping fragments amplified by PCR from several human cDNA libraries. In the course of this analysis, an alternatively spliced variant was identified that lacks exon 2 and is expressed at low levels (judged by PCR) along with the full-length mRNA in several tissues. Independently of this PCR results, the occurrence of an exon 2 deletion variant is confirmed by ESTs AW028814, AA836348 and BE301302.

A full-length cDNA containing the complete 120 kDa protein coding region was assembled using two overlapping PCR fragments containing exons 1.1 to 2.1 and 2.1 to 3.4, respectively (see, Nercc nucleotide coding sequence in SEQ ID NO:1). The cDNA encompassed the Nercc coding sequence with an ATG codon at nucleotides 1-3 of SEQ ID NO: 1 that defines an open reading frame encoding 979 amino acids (SEQ ID NO:2). Analysis of cDNA indicated that the Nercc translation initiation codon ATG is surrounded by a weak translational initiation consensus (Kozak) sequence, i.e., CCGCCATGT (SEQ ID NO:28). The Nercc coding region is followed by a 3′ untranslated region (defined by the cDNA DKFZp434D0935, accession code AL117502) with a polyadenylation signal, i.e., AATAAA (SEQ ID NO:29), followed downstream by a poly(A) tail.

Structural Analysis of cDNA-encoded Nercc1 Kinase

The predicted protein product has a calculated molecular mass of 107,034 Da, a theoretical pI of 5.50 and contains all 29 peptides detected in the mass spectroscopic analysis of the 120 kDa protein band. The polypeptide has a typical eukaryotic protein kinase domain situated near the N-terminus (amino acid residues 52-308 of SEQ ID NO:2) that exhibits all the features of a functional serine/threonine protein kinase. The activation loop is flanked by D₁₉₄YG and S₂₁₉PE motifs (amino acids 194-196 and 219-221 of SEQ ID NO:2, respectively), and contains several potentially phosphorylatable residues. The catalytic domain is most similar to the NIMA-related family of protein kinases (39-44% identity and 56-66% similarity with vertebrate Neks, 33% identity and 49% similarity with NIMA). As shown diagrammatically in FIG. 1, immediately following the catalytic domain (Protein Kinase) is a nuclear localization signal (NLS) composed of two classical nuclear localization motifs (amino acids ₃₀₆PLLRKRRR₃₁₃ and ₃₂₅PTKRPR₃₃₀ of SEQ ID NO:2). Thereafter is a domain containing seven consecutive RCC1 (regulator of chromosome condensation) repeats (amino acid residues 347-726 of SEQ ID NO:2), followed by a segment containing nine consecutive glycine residues (polyglycine region at amino acids 752-760 of SEQ ID NO:2), encompassed within a PEST region (amino acids 734-779 of SEQ ID NO:2); the polyglycine segment is likely to act as a flexible hinge. An acidic serine/threonine/proline-rich segment at amino acids 761-890 of SEQ ID NO:2 follows next and includes two motifs, which conform to the SH3 domain-binding “PXXP” motif (i.e., amino acids 823-830 and 881-888 of SEQ ID NO:2, respectively), and seven serine/proline (S/P) and threonine/proline (T/P) sites (four overlapping the latter two “PXXP” motifs). Immediately succeeding this region is a predicted coiled-coil domain (amino acids 891-940 of SEQ ID NO:2), followed by the protein carboxy terminus. This polypeptide is designated Nercc1 kinase, based on the similarity of the kinase domain to the NIMA/Nek kinases, and the presence of an RCC1 -like domain.

A striking difference between Nercc1 kinase, NIMA, and the Neks characterized thus far in higher eukaryotes is the presence in the Nercc1 kinase of a domain homologous to the RCC1 protein. RCC1 is a guanine-nucleotide-exchange factor for the small G-protein Ran, and is composed of seven repeats of 51-68 residues folded in a structure that resembles a seven-blade propeller (Renault et al., Nature, 392: 97-101 (1998)). The Nercc RCC1 domain has a 27% identity and 43% similarity to RCC1, and like RCC1 contains seven tandem repeats. Interestingly, a hypothetical protein kinase in the genome of Drosophila melanogaster (accession code AAF56344) exhibits an architecture homologous to Nercc kinase, i.e., an N-terminal NIMA-related protein kinase domain followed by a series of RCC1 domain repeats.

Although there is no significant identity in the primary sequences of the carboxy terminal noncatalytic segments of NIMA and the Nercc1 kinase, these two segments do share a number of related features, namely a nuclear localization signal (NLS) immediately following the catalytic domain, a proline-rich segment containing multiple S P and T P sites (some of which, in the case of NIMA, are probably phosphorylated during mitosis and appear to be important for regulation, see, Osamani and Ye, Biochem. J, 317: 633-641 (1996); Fry and Nigg, Curr. Biol., 5: 1122-1125 (1995)), and a coiled-coil domain (see, FIG. 1). In addition several PEST regions (involved in control of protein stability) are found in both protein kinases.

### Example 3

### Expression of Nercc mRNA and Polypeptide

More than 150 human ESTs corresponding to the 3′ untranslated segment of the Nerco cDNA are present in the NCBI database, as well as approximately 100 ESTs corresponding to the 5′ untranslated and coding region. The variety of tissues from which these ESTs were derived indicates that Nercc is widely expressed. In fact, Nercc DNA fragments were amplified by PCR of first strand cDNA prepared from human heart, brain, placenta, liver, skeletal muscle, kidney, and pancreas. Total mouse RNA isolated from various tissues, hybridized with a Nercc probe, demonstrating the expression of one or two Nercc mRNA species (5.3 and 8.1 kb) in all tissues examined.

Polyclonal anti-peptide antibodies, which were raised against synthetic peptides corresponding to Nercc amino acids 3-18 of SEQ ID NO:2 (N1 antibody) and 843-858 of SEQ ID NO:2 (C1 antibody), were used to detect the expression of recombinant and endogenous Nercc kinase polypeptides. Extracts prepared from HEK293 cells transfected with empty FLAG vector or vector encoding FLAG Nercc1 kinase fusion protein (“FLAG Nercc”) were probed with anti-Nercc N1 and C1 antibodies. Recombinant FLAG Nercc was easily visualized in immunoblots by both N1 and C1 antibodies. In addition, an endogenous immunoreactive band at 120 kDa was visualized with the antibodies. Immunoblots of other human (HeLa), murine NIH3T3) hamster (BHK), and marsupial (PtK2-Kangaroo rat) cell lines with anti-Nercc C1 antibody revealed, in each case, an immunoreactive band at 120 kDa A western blot prepared from multiple murine tissues probed with the C1 antibody exhibited a 120 kDa polypeptide, as well as the variable presence of an immunoreactive band at approximately 140 kDa, although the identity of the latter band was unknown.

### Example 4

### Nercc Kinase Interacts with Nek6

Inasmuch as the p120 Nercc kinase was identified as a protein that co-purified with the protein kinase Nek6, it was of interest to confirm this association by co-expression of a GST Nek6 fusion protein and a FLAG Nercc fusion protein in 293 cells. Several fusion proteins of full-length and truncated Nercc1 and Nek6 were prepared to test the interaction between Nercc and Nek6 using antibodies to FLAG and GST. Recombinant GST Nek6 fusion protein was affinity purified on glutathione (GSH)-agarose, and the eluate was probed with anti-FLAG antibody.

Full-length FLAG Nercc fusion protein was seen to bind specifically to the GST Nek6 fusion protein, whereas the carboxy terminally truncated FLAG Nercc (amino acids 1-739) fusion protein (despite comparable expression) was unable to bind the GST Nek6 fusion protein. Reciprocally, the fusion of the Nercc kinase carboxy terminal segment (Nercc amino acids 732-979 of SEQ ID NO:2) to GST was sufficient to enable specific binding of FLAG Nek6. Further analysis (see below) indicated that the site of Nek6/Nercc interaction lies between amino acids 732 and 891 of the Nercc amino acid sequence (SEQ ID NO:2).

### Example 5

### Nercc Homodimerizes Through a Coiled-coil Domain Distinct from Nek6 Binding Site

Nercc1 kinase contains a predicted coiled-coil motif near its carboxy terminus (amino acid residues 891-940 of SEQ ID NO:2), a likely candidate for an oligomerization domain as determined by Coils 2.1 software (FIG. 2A). The ability of Nercc to form homo-oligomers was shown by the co-precipitation of FLAG Nercc fusion with a HA Nercc fusion (FIG. 2B). A Nercc1 kinase segment (i.e., Nercc (891-940), referring to the portion of Nercc1 kinase consisting of amino acids 891-940 of SEQ ID NO:2) was fused to GST and co-expressed with either full length FLAG Nercc or a FLAG Nercc (1-891) in which the Nercc portion was deleted of its C-terminal 89 residues (i.e., to give the Nercc segment of amino acids 1-891 of SEQ ID NO:2). FIG. 2C shows that while full-length (FL) Nercc associated specifically with the GST Nercc (891-940) fusion, deletion of the carboxy terminal 89 residues of Nercc abolished this association (left panel). In addition, Nercc (1-891) cannot oligomerize with full-length Nercc (right panel). Thus, Nercc oligomerizes through its C-terminal coiled-coil domain. Additional work has shown that this oligomerization is important for the regulation of Nercc1 kinase activity.

Gel filtration analysis of both recombinant and endogenous Nercc proteins in 293 cells showed that the protein kinase exists in a high molecular weight complex of about 600 kDa. The larger-than-expected size probably reflects the association of Nercc with other proteins, such as Nek6, and perhaps higher order homo-oligomers and/or a complex of asymmetric shape. Deletion of amino acids 891-940 in the Nercc amino acid sequence of SEQ ID NO:2 did not affect Nek6 interaction with Nercc; the full length Nercc and Nercc (1-891), both interacted with Nek6 with similar affinities. Moreover, GST Nercc (891-940) did not bind Nek6 (data not shown). Thus, Nek6 binds to Nercc between amino acids 732 to 891 of SEQ ID NO:2, i.e., the region between the end of the Nercc RCC1 auto-inhibitory domain and the beginning off the coiled-coil domain.

### Example 6

### Analysis of the Protein Kinase Activity of Nercc

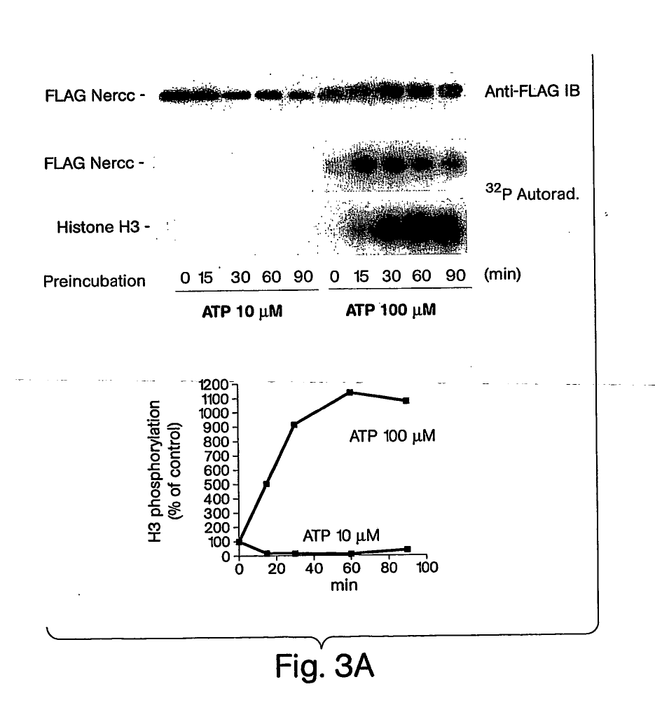

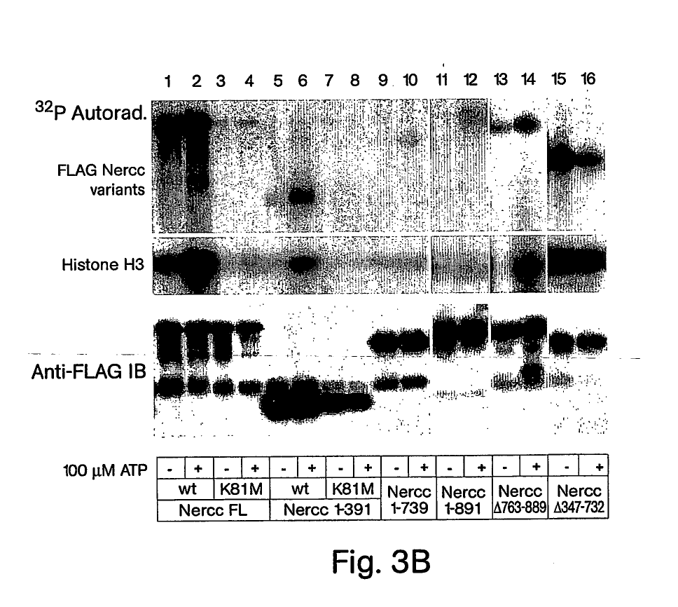

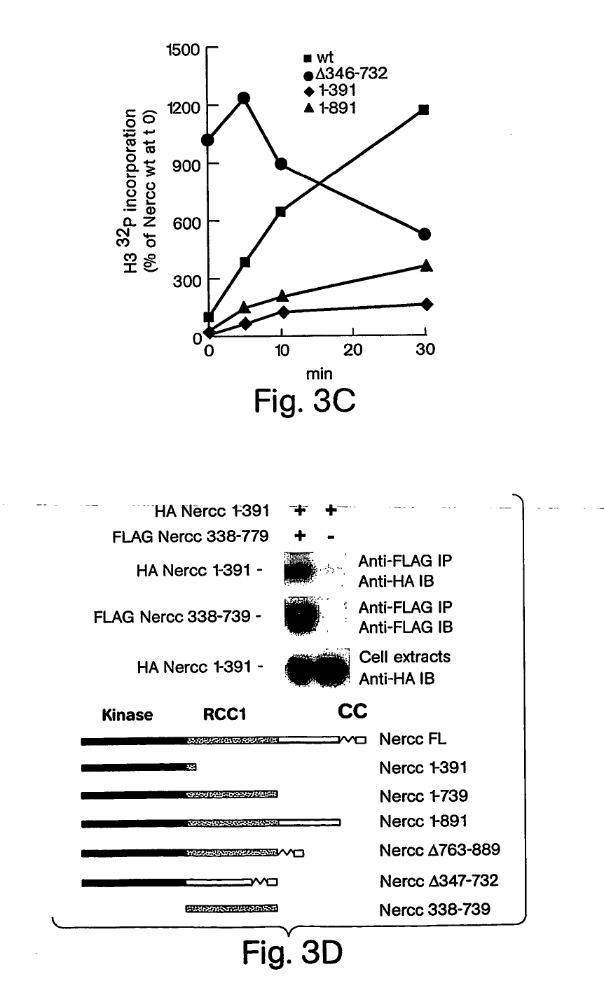

The catalytic properties of Nercc were studied using immunoprecipitates of FLAG-tagged forms of the Nercc polypeptide transiently expressed in HEK293 cells. The specificity of the measured protein kinase activity for the Nercc polypeptide was verified by the inability of an ATP-binding site mutant of Nercc (K81M) to catalyze significant ³²P transfer to itself or exogenous substrates (see, FIG. 3B, lanes 3, 4). Thus the kinase activity described below was due to Nercc and not to a contaminating protein kinase (e.g., Nek6).

Nercc can auto-phosphorylate and phosphorylate known in vitro kinase substrates, including different histones and MBP; the substrate β-casein was phosphorylated much less rapidly (not shown). Histone H3 was chosen as a model substrate. Phosphoaminoacid analysis of Nercc auto-phosphorylation and phosphorylation of histone H3 showed that Nercc is a Ser/Thr kinase. No phosphotyrosine was detected by either thin layer electrophoresis of partial acid hydrolysates of ³²P-Nercc or ³²P-histone H3 or by anti-phosphotyrosine immunoblot.

Recombinant wild type Nercc1 kinase had low basal activity when extracted from exponentially growing cells, however preincubation with Mg²⁺ plus ATP (100 μM) induced Nercc auto-phosphorylation (accompanied by a slowing in electrophoretic mobility) and activation (this activation could be reversed by phosphatase treatrnent, see below). The rate of in vitro activation was greatly enhanced if Mn²⁺ replaced Mg²⁺ in the phosphorylation reaction (not shown). Auto-phosphorylation/auto-activation is time- and ATP concentration-dependent; importantly, 10 μM ATP failed to enable significant Nercc activation even after incubation times of 90 minutes at 25° C., whereas 100 μM ATP gave maximal activation (10-20 fold) by 60 minutes, with half maximal activation at 20 minutes (FIG. 3A).

Once activation was complete, Nercc catalyzed a robust phosphorylation of histone H3 at 5-10 μM ATP. This apparent increase in affinity for ATP after activation enabled the design of a Nercc kinase assay that reflects the extent of activation achieved. Notably, endogenous Nercc, immunoprecipitated out of exponentially growing cells by specific antibodies, exhibited a similar pattern of Mg²⁺ ATP-dependent auto-activation in vitro.

Nercc was able to use GTP as a phosphate donor; GTP supports auto-activation, and after maximal auto-activation in vitro, enabled the phosphorylation of histone H3 at approximately 30% the rate observed with ATP.

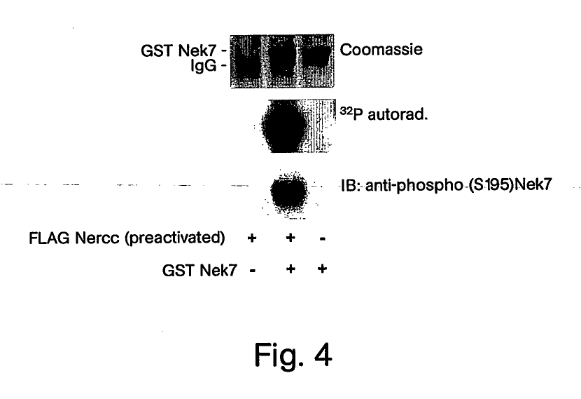

A structure-function analysis of the ability of recombinant Nercc1 kinase to auto-activate in vitro was also carried out. A series of Nercc variants, transiently expressed in HEK293 cells, were assayed for histone H3 kinase activity after preincubation for 30 minutes at 25° C. with Mg²⁺ alone or Mg²⁺ plus 100 μM ATP, the latter a condition sufficient to enable near-maximal auto-activation of wild type Nercc (FIG. 3A). After washing away the nonradioactive ATP, the histone H3 kinase assay was commenced using a concentration of γ-³²P-ATP (5-10 μM) below that capable of supporting auto-activation of wild type Nercc kinase (FIG. 3B, lanes 1 and 2). An ATP-binding loop-mutant of Nercc (K81M) showed no significant auto-phosphorylation/kinase activity, irrespective of preincubation with Mg²⁺ ATP (FIG. 3B, lanes 3 and 4). This was also true of Nercc (338-979), which lacked entirely the Nercc1 kinase domain (not shown). A truncated Nercc (1-391), which lacked the RCC1 domain and the C-terminal tail, showed a low basal protein kinase activity, but could be modestly activated by preincubation with Mg²⁺ and 100 μM ATP (FIG. 3B, lanes 5 and 6). However, a Nercc (1-391, K81M) was entirely devoid of activity (FIG. 3B, lanes 7 and 8), as was a Nercc (1-308), which terminated near the end of the canonical kinase domain. Nercc (1-739) retained both the protein kinase domain and the RCC1 domain, but was nevertheless inactive and incapable of auto-activation (FIG. 3B, lanes 9 and 10). Deletion of the coiled-coil domain, as in Nercc (1-889), diminished greatly the rate of auto-activation (FIG. 3B, lanes 11 and 12), whereas deletion of the proline-rich carboxy terminal segment while retaining the coiled-coil (Nercc Δ763-889) permitted substantial auto-activation (FIG. 3B, lanes 13 and 14). Selective deletion of RCC1 domain, i.e., Nercc (Δ347-732), resulted in a very high basal H3 kinase activity as compared with wild type Nercc, that was not further increased by preincubation in vitro with Mg²⁺ plus 100 μM ATP (FIG. 4b, lanes 15 and 16; FIG. 3C). A more detailed examination of the time course of activation by ATP (100 μM demonstrated that while both Nercc (1-391) and Nercc (1-889) were capable of auto-activation, the rate was greatly diminished as compared with wild type. Thus, deletion of the RCC1 domain produced a mutant with a basal activity similar to the maximal attainable by auto-activation (although the Nercc (Δ347-732) polypeptide is less stable at 25° C.), however, if the carboxy terminal dimerization domain was also deleted (as in Nercc (1-391)), the basal activity returned to low levels and auto-activation was severely retarded.

These results indicate a mechanism for Nercc1 kinase regulation (at least in vitro) in which the Nercc homodimer is maintained in an inhibited state by the ability of the RCC1 auto-inhibitory domain to abrogate intramolecular auto-phosphorylation. The inability of auto-activation to occur at ATP concentrations that enable robust phosphate transfer once activation has occurred is consistent with the view that the inhibitory action of the RCC1 domain operates, at least in part, by obstruction of the ATP binding site. One prediction of this model is that the RCC1 auto-inhibitory domain and the kinase domain of Nercc are likely to interact. The occurrence of such an interaction was shown in FIG. 3D; a FLAG Nercc (338-739) fusion protein expressed in HEK293 cells associated directly with co-expressed an HA Nercc (1-391) fusion, supporting the view that the RCC1 auto-inhibitory domain may inhibit the kinase domain through a direct interaction. Nercc auto-phosphorylation is likely to occur in trans within the homodimer, as deletion of the RCC1 domain results in spontaneous activation in vivo only if the carboxy terminal tail, i.e., the ability to homodimerize, remains intact.

### Example 7

### Production of Phospho-specific Antibodies That Specifically Bind Phosphorylated Nek7

The protein kinase Nek7 is phosphorylated by active Nercc at different sites, Serine195 among them; this phosphorylation activates Nek7. Polyclonal antibodies were produced against the peptide CAAHS*LVGTPYYM (were S* denotes a phosphoserine; SEQ ID NO:27), corresponding to the sequence surrounding serine 195 in mouse Nek7; the antibodies were designed to recognize Nek7 phosphorylated at Ser195, while not recognizing the unphosphorylated Nek7. FIG. 4 shows that when incubated with Mg²⁺/[γ-³²P]ATP plus active Nercc (preactivated by incubation in 100 μM ATP), bacterial recombinant GST Nek7 incorporated ³²P (middle panel, lane 2). This incorporation was not observed when GST Nek7 was incubated with Mg²⁺/[γ-³²P]ATP alone (middle panel, lane 3). Using the anti-(Ser195)Nek7 antibody, phosphorylated GST Nek7 was readily detected (lower panel, lane 2), while no cross-reaction with unphosphorylated GST Nek7 was observed (lower panel, lane 3). The results show that antibodies, such as the anti-phospho-(Ser195)Nek7 antibody, can thus be used to determine Nercc1 kinase activity using bacterial Nek7 as a substrate.

### Example 8

### Nercc1 Kinase Binds Ran

The presence in Nercc of a domain homologous to RCC1, a nucleotide exchange factor protein for the Ran GTPase, raises the question of whether Nercc binds Ran, and if so, to what functional effect. Prokaryotic recombinant GST and GST-fusions with the Nercc1 kinase domain (GST Nercc (1-391)), the RCC1 domain (GST Nercc (338-739)) and C-terminal tail (GST Nercc (732-979)) were immobilized on GSH-agarose beads, and incubated with prokaryotic recombinant Ran which had been preloaded with GTPβS or GTPγS. Whereas neither GST nor GST Nercc (732-979) bound Ran, the GST Nercc kinase domain and RCC1-like domain fusion proteins were able to bind Ran with very high efficiency. Both Nercc domains bound Ran-GDP to a somewhat greater extent than Ran-GTP.

In an effort to examine the interaction between Ran and Nercc in vivo, HA wild type Ran(wild type) was-co-expressed with different FLAG Nercc mutants, and the FLAG immunoprecipitates were probed for the presence of HA wild type Ran. Cell lysis and subsequent washes were carried out in the presence of excess Mg²⁺ to conserve Ran in its nucleotide bound form. Wild type Ran associated with full length Nercc as well as with the isolated Nercc catalytic domain fragments, Nercc (1-308) and Nercc (1-391). The deletion of the RCC1 domain, as in the mutant variant Nercc (Δ347-732), or the C-terminal tail, as in variant Nercc (1-739), did not detectably impair the binding of HA Ran, and in contrast to the in vitro results, the isolated Nercc RCC1-like domain (Nercc (338-739)) exhibited very little association with Ran.

A potential confounding element in assessing the interactions of Nercc with Ran in vivo was the subcellular localization of the wild type and mutant polypeptides. In interphase cells Ran GDP is located exclusively in the cytoplasm whereas Ran GTP is restricted to the nucleus, a situation maintained by the nuclear location of the Ran GEF, RCC1 and the cytosolic localization of Ran GAP. Nercc (1-391), which was exclusively nuclear, clearly associated with HA wild type Ran, as did Nercc wild type and Nercc (Δ338-732), both of which were exclusively cytoplasmic. In addition, a nuclear form of full length Nercc created by the addition of an NLS peptide to the Nercc N-terminus (NLS Nercc FL) bound less Ran than did Nercc wild type expressed at similar levels. These results indicated that the Nercc catalytic domain binds Ran in vitro and in vivo regardless of its subcellular localization. The specificity of the interaction between Ran and the Nercc catalytic domain was assessed by examining the relative ability of Ran to bind the Nercc catalytic domain Nercc (1-308) or Nek 6, another protein kinase in the NIMA subfamily; no binding of HA Ran to Nek6 was detectable, whereas, both the Nercc catalytic domain and RCC1 (the Ran GEF, a positive control) bound avidly to Ran.

In summary, Nercc binds Ran specifically; binding is certainly mediated by the Nercc1 kinase catalytic domain and by the RCC1 domain as well. The catalytic domain binds Ran-GDP in preference to Ran-GTP, and this may be true for the RCC 1 domain at least in vitro.

To address more directly the relative binding in vivo of Nercc to Ran-GDP compared to Ran-GTP, the cytoplasmic wild type Nercc and a nuclear form of full length Nercc (NLS-Nercc) were each co-expressed with wild type Ran or Ran mutants that bind GDP (T24N) or GTP (G19V) exclusively; either Ran or Nercc was immunoprecipitated and probed for the association with the other polypeptide. The full-length wild type Nercc bound co-expressed wild type Ran and Ran T24N to a greater extent than RanG19V. Although this suggests a preference for Ran-GDP, it should be noted that whereas RanT24N and Nercc are both cytoplasmic, RanG19V is exclusively nuclear in localization. However NLS-Nercc which is exclusively nuclear, nevertheless also did not bind the GTP-locked mutant RanG19V, strongly supporting the conclusion that full length Nercc indeed has a higher affinity for Ran-GDP over Ran-GTP. The Nercc catalytic domain (amino acids 1-308 of SEQ ID NO:2), which is distributed both in the cytosol and the nucleus during transient expression, also showed little or no binding to Ran (G19V). Regarding the Nercc RCC1 domain (Nercc 338-739), co-expression with wild type Ran, Ran T24N, or Ran G19V did not result in the recovery of Ran polypeptides; whether Nercc (338-739) was cytoplasmic or directed to the nucleus by fusing it to Nercc NLS did not affect this result.

The inability of the Nercc RCC1 domain to bind Ran in vivo is as yet unexplained. Nercc (338-739) is exclusively cytoplasmic and is well expressed; the low binding of the Neroc RCC 1-like domain to Ran-GDP in vivo is not therefore attributable to mislocalization. The RCC1-like domain may simply bind Ran with lower affinity than the catalytic domain; conversely, access of Ran to the isolated Nercc RCC1-like domain in vivo may be obstructed.

The effect of Ran on Nercc activation in vitro was examined by preincubation of Nercc with Mg and GST or GST-Ran, GST-Ran (GDPβs) or GST Ran (GMPPNP), followed by the addition of nonradioactive ATP (100 μM). At intervals thereafter, aliquots were removed, mixed with [γ-³²P] ATP and histone H3, and the incorporation of ³²P into H3 was measured during a further 5 minutes incubation. In this manner, Ran was observed to cause a modest increase in the rate of Nercc kinase activation. This effect was independent of Ran nucleotide charging, and was also observed with Ran treated with EDTA prior to addition of Nercc and thus presumably nucleotide-free.

### Example 9

### Nercc is Phosphorylated and Activated During Mitosis and Can Be Phosphorylated In Vitro by p34 ^{Cdc2}

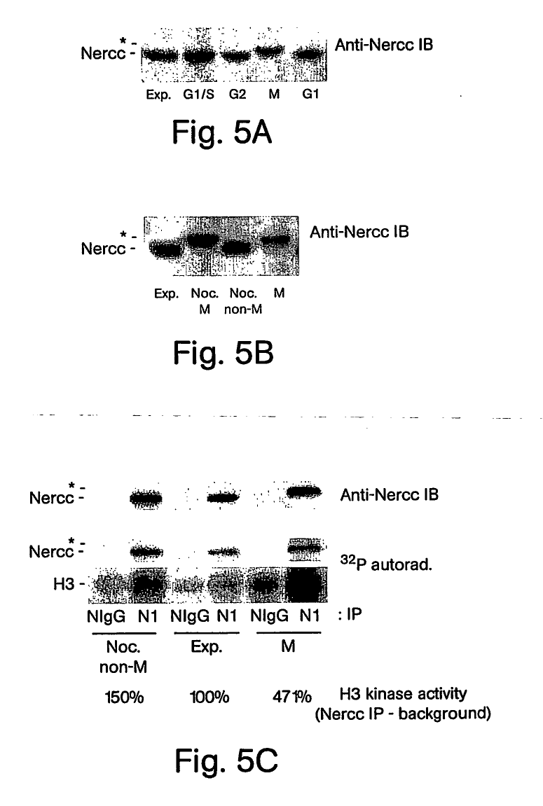

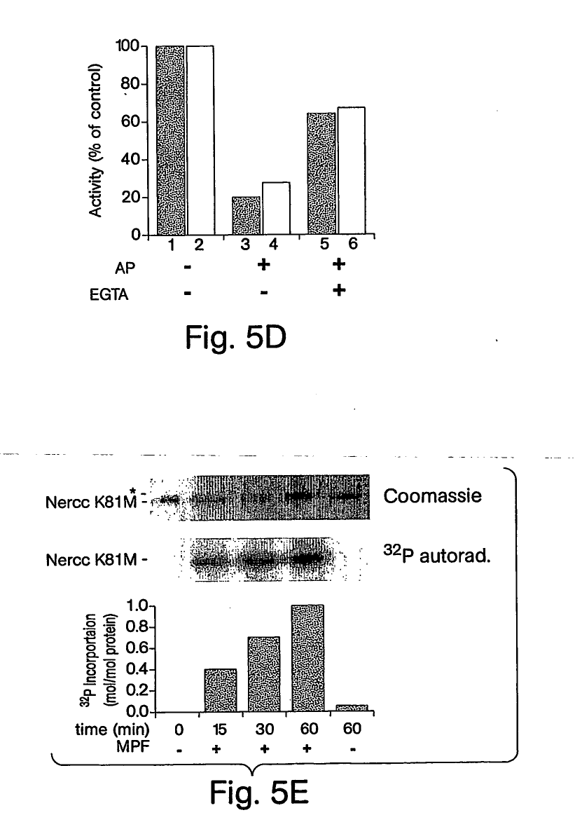

In view of the structural similarities between Nercc and the NIMA protein linase, it was of interest to determine whether Nercc1 levels or activity changed during cell cycle progression. Extracts prepared from HeLa cells collected at different phases of the cell cycle (G1/S, G2, M, G1), were immunoblotted for Nercc polypeptide, and Nercc kinase activity was assayed after immunoprecipitation. Nercc1 level remained constant during the phases of the cell cycle examined. However, the Nercc polypeptide displayed a marked slowing in electrophoretic mobility during mitosis (FIG. 5A), which could be mimicked by treatment in vivo with the protein phosphatase inhibitor calyculin (not shown). A similar electrophoretic slowing of Nercc occurred in CHO-K1, COS7, U2OS or HEK293 cells arrested in mitosis (not shown).

To determine whether Nercc modification occurs during normal mitotic progression or results from activation of a mitotic arrest checkpoint by the compound nocodazole, mitotic cells were collected by shake-off from a culture that had been pseudo-synchronized in G1/S by thymidine block and then released into the cell cycle; at the time of harvest, these cells were progressing normally through mitosis, enabling a comparison of Nercc in these mitotic cells with Nercc in nocodazole-treated cells, both mitotic and non-mitotic (FIG. 5B). The electrophoretic migration of Nercc was unmistakably upshifted in the mitotic cells, whether normally cycling or nocodazole-arrested, establishing that the Nercc modification is a characteristic of cells progressing normally through mitosis, rather than the result of activation of the spindle checkpoint.

In further experiments, mitotic nocodazole-arrested cells were used as a model of normal mitotic cells. Endogenous Nercc kinase activity was assayed in immunoprecipitates prepared from HeLa cells, comparing cells growing exponentially to cells treated with nocodazole; the latter were divided into those detached after mitotic shake-off (mitotic cells) versus those that remain attached (non-mitotic cells). The Nercc kinase activity in mitotic cells was 4 to 5-fold higher than in exponentially growing cells, despite comparable polypeptide levels; nocodazole-arrested, non-mitotic cells showed a small increase in Nercc kinase activity, probably due to contaminating mitotic cells (FIG. 5C). Similar results were obtained with U2OS cells (not shown). Thus, these data indicate that Nercc is activated during mitosis.

To determine whether the observed mitotic Nercc activation and change in electrophoretic mobility were due to phosphorylation, Nercc, which was immunopurified from mitotic cells, was incubated with phosphatase. As a control, recombinant FLAG Nercc fusion protein preactivated by incubation with 100 μM ATP was also treated with phosphatase (FIG. 5d). Phosphatase treatment increased FLAG Nercc electrophoretic mobility, and concomitantly reduced Nercc1 kinase activity against exogenous substrates, demonstrating that Nercc activity depends on phosphorylation. When endogenous mitotic Nercc was incubated with phosphatase, a similar decrease in protein linase activity was observed. The results indicate that Nercc activation in vitro and in vivo during mitosis is due to phosphorylation.

Transiently expressed, catalytically inactive forms of recombinant Nercc variants (Nercc K81M or Nercc 338-979) exhibited an upshift in electrophoretic migration on SDS-PAGE in response to nocodazole, much the same as wild type Nercc (not shown). Such inactive Nercc mutants cannot auto-phosphorylate, indicating that the mitotic modification of Nercc at least in part was due to another protein linase. In view of these data, a prinicipal candidate was p34^{Cdc2}.

Recombinant full-length FLAG Nercc mutant variant (K81M), eluted from anti-FLAG-agarose beads by competition with FLAG peptide, was phosphorylated in vitro by purified Xenopus active p34^{Cdc2}/cyclin B (MPF) (FIG. 5E). MPF-catalyzed Nercc phosphorylation induced an upshift in Nercc electrophoretic migration that was similar to that observed in vivo in mitotic cells. Furthermore, overall ³²P incorporation into Nercc (K81M) rapidly approached 1 mole PO₄/mole protein (FIG. 5E). Similar results were obtained with cyclin B-immunoprecipitates from nocodazole-arrested mitotic HeLa cells, whereas cyclin B-immunoprecipitates from non-mitotic cells did not catalyze Nercc phosphorylation (not shown). Inhibition of the Nercc-phosphorylating activity in the cyclin B immunoprecipitate from mitotic cells by the CDK inhibitor roscovitine confirmed the identity of the kinase activity as Cdc2.

These data show that Nercc is an in vitro substrate for p34^{Cdc2}, and this phosphorylation produces a change in Nercc electrophoretic mobility similar to that observed in mitotic cells, indicating that p34^{Cdc2} contributes to Nercc phosphorylation during mitosis. It is important to point out that phosphorylation of Nercc by Cdc2/MPF in vitro does not significantly alter maximal Nercc kinase activity toward histone H3. Similarly, the upshift in Nercc electrophoretic mobility that occurs on treatment of cells with calyculin is not accompanied by an increase in Nercc kinase activity (data not shown). Thus, although the auto-phosphorylation/auto-activation of Nercc in vitro is also accompanied by a slowing in electrophoretic mobility, the occurrence of an upshift should not be considered synonymous with Nercc activation. The details of the functional consequences of Cdc2-catalyzed phosphorylation of Nercc during mitosis require further study.

### Example 10

### Nercc Cellular Localization During Interphase and Mitotic Progression

Immunofluorescence studies using the N1 or C1 anti-Nercc peptide antibodies (see, above) showed Nercc to have a finely granular cytoplasmic fluorescence in all cell lines immunoblotted (except BHK cells, where Nercc immunoreactivity is too low for analysis) as well as in CHO-IR cells. Nercc appears distributed diffusely in the cytoplasm without association with organelles, plasma membrane or cytoskeletal elements. To confirm this, nonequilibrium sucrose density gradient fractionation of HEK293 cells was carried out. Nercc sedimentation corresponded to that of other cytoplasmatic proteins (e.g., lactate dehydrogenase), and was distinct from a variety of membrane markers, e.g. PCOP, a TGN marker. Overlay of Nercc-enriched fractions with lighter sucrose followed by centrifugation to equilibrium did not result in Nercc translocation up into the low density fraction, as occurs with membrane-bound proteins. Thus Nercc is localized in the cytoplasm and unattached to cellular membranes.

Notably, Nercc immunoreactivity is absent from the nucleus. The lack of nuclear Nercc was also evident on overexpression of recombinant FLAG Nercc. This was surprising, as Nercc contains a classical nuclear localization signal (NIS) that is fully functional when appended to the Nercc aminoterminus or to another polypeptide. The absence of Nercc in the nucleus might be due to a cell-cycle dependent nuclear import, nuclear import that depends on external stimuli, or strong nuclear export of Nercc. However, careful examination of immunofluorescence specimens of cells subjected to a variety of treatments failed to uncover instances of endogenous Nercc in nuclei. By example, exposure of HeLa cells to protein kinase inhibitors or CHO-IR cells to serum-starvation or treatment with 100 nM insulin never resulted in observable nuclear Nercc. To test whether the cytoplasmic localization of Nercc depended on strong nuclear export mediated by CRM1, BHK cells were transfected to express with wild type Nercc and exposed to leptomycin B (2, 20 and 200 nM) for periods ranging from 30 minutes to 24 hours. Nercc remained cytoplasmic, whereas endogenous RanBP1 fluorescence in the nucleus increased dramatically after 30 minutes incubation with 20 nM Leptomycin B. A similar lack of response to leptomycin B was also observed with endogenous Nercc in HeLa cells. Thus, Nercc is constitutively cytoplasmic in interphase cells, indicating that the Nercc NLS is not exposed to the cellular environment during interphase.

Nercc localization in mitotic cells was also examined. HeLa cells were blocked in mitosis with nocodazole, or enriched in mitotic cells by double thymidine block followed by release and mitotic shake-off. In both instances, Nercc immunoflourescence in mitotic cells was diffusely distributed throughout the cell and conspicuously absent from the chromosomes.

To test whether Nercc is associated with the mitotic spindle, mitotic HeLa cells were fixed after treatment for one minute with the non-ionic detergent saponin. Eg5, a microtubule-binding motor, is readily visualized on spindle microtubules using a specific anti-Eg5 antibody after this light saponin treatment, whereas endogenous Nercc immunofluorescence is completely removed from the cells under these conditions. Thus, Nercc is not tightly associated with the spindle microtubules, and is diffusely distributed through the cell during mitosis.

A series of FLAG Nercc mutants were also examined for their cellular distribution during transient expression in HeLa cells. Four types of subcellular distribution were observed: cytoplasmic, nucleocytoplasmic, predominantly nuclear, and exclusively nuclear. Overexpressed wild type Nercc was distributed in the cytoplasm like endogenous Nercc. However, about 5% of cells exhibited slight nuclear immunofluorescence. The inactive Nercc ATP site mutant (K81M) exhibited a substantial nuclear component, suggesting that the nuclear localization site (NLS) might be inactivated by auto-phosphorylation. Nercc (1-391), although potentially activatable, was nevertheless exclusively nuclear; this established the functionality of the Nercc NLS, as did the exclusive nuclear localization of a wild type Nercc to which a copy of its endogenous NLS was fused at its amino terminus.

The conversion of Nercc to forms that achieve nuclear localization results in the frequent occurrence of micronuclei, multiple nuclei, and lobed nuclear morphologies. Such phenotypes have been associated in other circumstances with lagging chromosomes and chromosome non-disjunction in anaphase (Cimini et al., J. Cell Biol., 153: 517-527 (2001)). The frequency of these morphologies was highest in cells expressing a Nercc mutant variant (amino acids 1-391), i.e., an active kinase domain, with the nuclear localization. However, expression of the cytoplasmic kinase-inactive variant Nercc (1-739) as well as the nuclear/cytoplasmic variants Nercc (K81M) and Nercc (1-308) have a similar, but less marked effect.

### Example 11

### Nercc1 Kinase Regulates Mitotic Progression

The structural similarities between Nercc and NIMA, the activation of Nercc during mitosis, the ability of Nercc to bind Ran in vitro and in vivo, and to induce abnormal nuclear morphology when targeted to the nucleus, pointed to the likelihood that Nercc is a mitotic regulator. A preliminary indication was provided by the observation that transfection of plasmid encoding an eGFP Nercc (K81M) fusion protein appeared to interdict cell division. Thus, time-lapse recordings of transfected HeLa cells showed that, whereas 78% of all (n 70) cells transfected with plasmid encoding eGFP alone underwent division within the subsequent 36 hours, only 4% of all (n=52) HeLa cells transfected with eGFP-Nercc (K81M) underwent division, and 85% proceeded to cell death as compared with 18% of the eGFP-transfected cells. Interestingly, even wild type Nercc was somewhat toxic: only 29% of all (n=31) HeLa cells transfected with an eGFP-Nercc plasmid proceeded through mitosis. The impact of the nuclear-localized mutant variant Nercc (1-391) was even more marked; only 1% of all (n=45) HeLa cells transfected with eGFP-Nercc (1-391) underwent division, and 72% of these cells were dead after 36 hours.

To test directly whether Nercc participates in the control of mitotic progression, real-time observations were made of PtK2 cells after microinjection of affinity purified anti-Nercc (peptide) antibodies or purified preimmune IgG. When Ptk2 cells were injected with Nercc antibody during interphase, mitosis was never subsequently observed. Therefore, antibody microinjection of cells in prophase was undertaken, in which case prophase cells were identified by the presence of chromosome condensation and nucleolar disassembly. All such cells microinjected with normal rabbit IgG (5 at 2.5 mg/ml and 15 at 10 mg/ml) completed mitosis normally and produced daughter cells, save one, which had a lagging chromosome; this occurrence was consistent with the frequency of lagging chromosomes reported earlier for PtK cells (Izzo et al., Mutagenesis, 13: 445-451 (1998)). In contrast, 14 cells of 30 (i.e., roughly 45%) cells microinjected with affinity-purified anti-Nercc (C1 antibody) IgG (2.5 mg/ml) showed mitotic abnormalities of two basic types. The first type of mitotic abnormality involved abnormalities in chromosomal segregation associated with abnormal spindle dynamics (FIG. 6A, Panels A and B). Thus, four cells never entered anaphase B, (i.e., movement of poles with attached chromosomes in opposite directions), although the spindle was apparently normal; nevertheless, cytokinesis proceeded. In two experiments, this resulted in the subsequent trapping of one or more chromosomes in the cytokinetic furrow, creating DNA bridges between the nuclei of daughter cells. In two other cases, separation of the chromosomes to opposite poles stopped prematurely, chromosome decondensation was observed, and a cytokinetic arrow formed to one side of the spindle, resulting in formation of one daughter cell with 4N DNA and one daughter cell without DNA. The second type of mitotic abnormality that was observed affected the formation of the mitotic spindle (FIG. 6A, Panel C). In eight cells, the spindle was not visible on phase contrast images throughout the whole duration of the recording, and approximately 40 minutes after the nuclear envelope breakdown the chromosomes concentrated in the center of the rounded-up PtK2 cell, with chromosome arms extending far into the cell periphery. When fixed immediately after the recording, these cells showed a highly disrupted spindle. Although two separated centrosomes were visible, the cells showed interphase arrays of microtubules (FIG. 6B, anti-Nercc).

Two anti-Nercc C1-microinjected cells formed a bipolar mitotic spindle, but anaphase started prematurely, specifically at only 8 minutes after the last mono-oriented chromosome acquired bi-orientation. This was 1 minute less than the minimum time recorded for PtK cells (Rieder et al., J. Cell Biol., 127: 1301-1310 (1994)). Thus, in these cells, anaphase started without congression of chromosomes to the metaphase plate, although cell division proceeded normally thereafter.

Similar kinds of mitotic abnormalities, including a failure to enter anaphase B, were observed in 4 of 8 cells microinjected with an anti-Nercc IgG raised against a peptide sequence from the Nercc catalytic domain (E2 antibody, to Nercc amino acids 80 to 94 of SEQ ID NO:2). Microinjection of ten Ptk2cells with affinity-purified anti-Nercc N1 (Nercc amino acids 3-18 of SEQ ID NO:2) IgG did not alter the normal progression of mitosis.

In another study, the Nercc C-terminus antibody was also microinjected into CF-PACL cells in prophase. CF-PAC1 cells are a human cell line that contains levels of Nercc comparable to HeLa and HEK 293 cells and which has been employed previously for microinjection studies of spindle dynamics (Mountain et al., J. Cell Biol., 147: 351-365 (1999)). Three out of five microinjected cells arrested in prometaphase for 3-10 hours with several mono-oriented chromosomes close to the spindle poles. In contrast, all 5 control cells microinjected with normal rabbit IgG showed normal mitosis. Only one of five CF-PAC1 cells microinjected early in prometaphase with anti-Nercc antibody showed a similar mitotic defect, whereas the other four proceeded through mitosis normally.

The occurrence of similar mitotic abnormalities in Ptk2 cells microinjected with two independently prepared anti-Nercc antibodies, raised against different Nercc peptides, and the absence of these phenotypes in response to nonimmune IgG, together with the occurrence of similar abnormalities in CF-PAC1 cells indicate strongly that interference with Nercc is the basis for these phenotypes. In summary, microinjection of anti-Nercc (C-terminal peptide) C1 IgG and anti-Nercc (catalytic domain peptide) E2 IgG results in the frequent occurrence of abnormal spindle dynamics and abnormal chromosomal segregation, indicating that Nercc ordinarily participates in the regulation of these processes.

### Example 12

### Studies of the Involvement of Nek6 and Nek7 Proteins in Mitosis

To gain insight into Nek6/Nek7 regulation and function, studies were carried out to elucidate the mechanism underlying the activation of the Nek6 and Nek7 kinases and to define the regulation of the endogenous enzymes. The studies described below indicate that Nercc1 kinase, previously shown to bind Nek6 (see, Examples 2 and 4, above), phosphorylates directly a critical site on the activation loop of both Nek6 and Nek7 and activates these kinases in vitro and in vivo. Moreover, like Nercc1 kinase, endogenous Nek6 is activated in mitosis. The ability of Nercc1 kinase to directly activate Nek6 points to the operation of a cascade of NIMA-related mitotic protein kinases.

Nek6 (together with its close homolog, Nek7) is purified from rat liver as the predominant kinase capable of phosphorylating in vitro the hydrophobic regulatory site (Thr412) of the p70 S6 Kinase (p70S6K) in vitro as previously described (Belham et al., Curr. Biol., 11: 1155-1167 (2001)). Recombinant Nek6 polypeptide is recovered as an active protein kinase after transient expression in HEK293 cells and activates coexpressed p70 S6 Kinase in vivo (as well as directly in vitro) in a manner synergistic with PDK1. Nevertheless recent findings indicated that Nek6. (and by extension Nek7) is not a physiologic activator of p70 S6 Kinase (Lizcano et al., J. Biol. Chem., 277: 27839-27849 (2002)), and thus its roles in cell regulation remained unknown.

Materials and Methods

Expand HiFidelity DNA polymerase, sequencing grade trypsin and COMPLETE™ protease inhibitor cocktail tablets were purchased from Roche. Protein A and G-Sepharose and GSH-Sepharose were from Amersham (Piscataway, N.J.). Insulin, rapamycin, worttnannin, nocodazole, and FLAG M2 antibody were obtained from Sigma (St. Louis, Mo.). Anti-myc (9E10) monoclonal antibody, anti-cyclin B1, and rabbit anti-p70 polyclonal antibody (C-18) were purchased from Santa Cruz (Santa Cruz, Calif.). Anti-β tubulin was obtained from Zymed (South San Francisco, Calif.). LIPOFECTAMINE® transfection reagent, pcDNA3.1-myc/6His mammalian expression vector, and all cell culture media were GIBCO brand (Invitrogen, Carlsbad, Calif.), except phosphate-free DMEM was obtained from ICN (Irvine, Calif.). Cellulose thin-layer chromatography (TLC) plates were bought from E.M. Sciences (Gibbstown, N.J.). Rabbit polyclonal sera raised against KLH-coupled peptide were generated at Cocalico Biologicals (Rearnstown, Pa.). ³²P-labeled orthophosphate was NEN brand (PerkinElmer Life Sciences, Boston, Mass.) and [γ-³²P] ATP was obtained from ICN (Irvine, Calif.). MBP was purchased from UBI. HEK 293, H4-II-E-C3, HeLa and U2OS cells were obtained from the American Type Culture Collection (ATCC; American Type Culture Collection, Manassas, Va.).

Construction of Expression Plasmids and Recombinant Fusion Proteins

As with the studies with Nercc1 (see, Example 1, above), a number of derivative proteins (fusion proteins, mutant variants) of Nercc1, Nek6, and Nek7 linase proteins were made and employed to carry out the studies described here. The mammalian expression vectors PEBG 2T and pCMV5 FLAG encoding wild type and “kinase dead” (K74/75M) Nek6, the pCMV5 FLAG plasmid encoding different forms of Nercc1 kinase, and the bacterial expression vector pGEX KG expressing GST Nercc1 (732-979) have been previously described (see, Example 1, above; Roig et al., Genes Dev., 16 1640-1658 (2002); Belham et al., Curr. Biol., 11: 1155-1167 (2001); Kameshita and Fujisawa, Anal. Biochem., 183: 139-143 (1989)). For construction of pcDNA3.1 6His/myc Nek6, a PCR fragment was generated using pCMV5 FLAG Nek6 as template encoding the open reading frame of human Nek6 lacking the stop codon and surrounded at the initiator methionine by an optimum Kozak motif. This fragment was subcloned into pcDNA3.1 myc/6His allowing Nek6 to be expressed in mammalian cells as a fusion protein with 6His and myc epitopes at the carboxy terminus.

All site-directed mutant Nek6 variants in pcDNA3.1 myc/6His were constructed by PCR-mediated overlap extension mutagenesis and subsequently subcloned into pcDNA3.1 myc/6His. All clones were verified by sequencing.

Generation of Rabbit Anti-Nek6 Antisera

A synthetic polypeptide corresponding to the amino terminal sequence of mouse Nek6 (AGQPSHMPHGGSPN (SEQ ID NO:30)) conjugated to the carrier protein keyhole limpet hemocyanin (KLH) via a cysteine residue was used to immunize rabbits in order to produce anti-total Nek6 antibodies. Phosphospecific antibodies (“anti-P” antibodies) were produced using the following two synthetic phosphopeptides comprising Nek6 amino acids 194-205 and 203-214 of SEQ ID NO:4 and a standard protocol (see, Weng et al., J. Biol. Chem., 273: 16621-16629 (1998)):

CGRFFSSETT*AAH (SEQ ID NO:31), comprising an amino terminal cysteine (C) and Nek6 amino acid 194-205 of SEQ ID NO:4, phosphorylated at the carboxy proximal threonine (T*) residue (corresponding to T202 of SEQ ID NO:4) to produce anti-P T202 Nek6 antibodies;

CAAHS*LVGTPYYM (SEQ ID NO:27), comprising an amino terminal cysteine (C) and Nek6 amino acid residues 203-214 of SEQ ID NO:4, phosphorylated at the serine (S*) residue (corresponding to S206 of SEQ ID NO:4) to produce anti-P S206 Nek6 antibodies.

Cell Culture, Expression and Purification of Recombinant and Endogenous Proteins

Maintenance of HEK 293 cells and H4-II-E-C3 cells has been described previously (see, Belham et al., Curr. Biol., 11: 1155-1167 (2001)). HeLa cells were maintained in DMEM media plus 10% sera. HEK293 cells were transiently transfected using LIPOFECTAMINE® transfecting reagent according to the manufacturer's protocol. Cells were lysed in lysis buffer or buffer A (20 mM Tris pH 7.6, 2 mM EGTA, 1 mM EDTA, 5 mM MgCl₂, 20 mM β-glycerophosphate, 0.5% Triton X-100, 1 mM sodium orthovanadate, 1 mM DTT) supplemented with Calyculin A (50 nM) and COMPLETE™ protease inhibitor cocktail (1 tablet/50 mls). Lysates were centrifuged at 13,000× g for 30 minutes at 4° C.

To immunoprecipitate recombinant proteins, the corresponding antibodies prebound to protein A/G Sepharose were incubated with the lysates at 4° C. The imnuunoprecipitates (IP) were washed and subsequently used as indicated.

GST Nercc (732-979) and Nek6 expression in the E. coli strain BL21 DE3 pLys (Novagen, Madison, Wis.) transformed with the pGEX KG expression vector was induced by incubation with 300 μM IPTG for 16 hours at 25° C. The purification of this, and other recombinant GST fusion polypeptides from transfected mammalian cells was carried out using GSH-beads using standard protocols.

“In-gel” Kinase Assay

In-gel kinase assay of Nek6 activity was carried out according to Kameshita and Fujisawa (Anal. Biochem., 183: 139-143 (1989)). When indicated, 0.2 mg/ml of MBP was included in the gel as a substrate.

MS Determination of Phosphorylation Sites (“P” Sites)

Gel bands were excised as digested with trypsin and resultant peptides were extracted from the gel. An aliquot of peptide was loaded onto a fused silica (360 um OD, 50 um ID; PolyMicro) capillary C-18 (ODS) column with a pulled emitter tip (Martin et al., Anal. Chem., 72: 4266-4274 (2001)). Peptides were gradient-eluted from the HPLC (0-100% B where A=0.1% TFA and B=70% CH₃CN and 0.1% TFA) and analyzed by a LCQ DECA XP ion trap mass spectrometer (ThermoFinnigan, San Jose, Calif.). The mass spectrometer was set to data dependent mode to take MS/MS spectra of the top 5 most abundant m/z peaks in each MS scan. The MS/MS spectra were searched (Sequest; ThermoFinnigan, San Jose, Calif.) against a database containing the protein in order to deduce the sequences. Potential post-translational modifications were also searched [STY=80 (phosphorylation)].

An additional aliquot of each sample was subjected to immobilized metal affinity chromatography (MIAC) to enrich the sample for phosphorylated peptides. Each experiment was performed as previously described (Ficarro et al., Nature Biotech., 20: 301-305 (2002)). Briefly, peptides were loaded onto a capillary IMAC column. The column was washed to remove non-specific binding and then the peptides are eluted onto a capillary C-18 precolumn. The precolumn was washed with HPLC buffer and then connected to the analytical column described above. The peptides were eluted and analyzed as described above. Sequest peptide sequences were manually confirmed to ensure correct sequence identification.

Nek6 Kinase Assay

Immunoprecipitates (IP) of recombinant Nek6 (using anti-FLAG or anti-myc antibodies) or endogenous Nek6 were washed three times in the extraction buffer containing 0.5 M LiCl, and twice in kinase buffer (50 mM MOPS pH 7.4, 10 mM MgCl₂, 2 mM EGTA, 20 mM β-glycerophosphate). Kinase assays were performed in 30 μl kinase buffer containing 1.5 μg GST-p70S6K ΔCT104 (Thr252Ala) or MBP (0.2 mg/ml) and initiated by addition of [γ-³²P] ATP (5 μM final concentration at 4000 cpm/pmol). Reactions were incubated for 10 minutes at 30° C. and terminated by the addition of 4×SDS sample buffer. Kinase mixtures were resolved by SDS-PAGE, transferred to PVDF membrane, and Nek6 phosphotransferase activity determined by excising Coomassie stained bands corresponding to the substrate and measuring radioactive content by Cerenkov scintillation counting.

Two Dimensional (2D)-tryptic Phosphopeptide Mapping

HEK293 cells expressing various Nek6-His6/myc constructs were incubated with phosphate free DMEM containing ³²P for 4 hours prior to harvest. The myc-immunoprecipitates (IP) were subjected to SDS-PAGE, fixation, and staining. The gel slice containing 32P-Nek6 was equilibrated in 50 mM ammonium bicarbonate buffer (pH 8.5), homogenized, and subjected to several rounds of tryptic digestion until at least 75% of initial ³²P-Nek6 was extracted into the supernatant. The dried, salt-free digest was separated by thin layer electrophoresis (TLE) at pH 1.9, followed by TLC as described previously (Boyle et al., Methods Enzymol., 201: 110-149 (1991)). Plates were exposed using a PhosphorImager imaging system.

Quantitative PCR

Total RNA was extracted using TRIzol (Invitrogen, Carlsbad, Calif.), further purified using the Qiagen (Valencia, Calif.) RNEASY® total RNA isolation kit according to the instruction manual and quantified using Ribogreen (Molecular Probes, Eugene, Oreg.). After DNase 1 treatment, QRT-PCR was performed in duplicate using the Brilliant One-Step QRT-PCR kit (Stratagene, La Jolla, Calif.) containing SYBR Green I (1:30,000, Sigma, St. Louis, Mo.), forward and reverse primers (25 nM each), and sample RNA (1 μg). The nucleotide sequence of the primers used were:

| | |
|---|---|
| Nek6-F primer | |
| (CGAAAAGAAGATAGGCCGAGG) | (SEQ ID NO:32) |
| and | |
| Nek6-R primer | |
| (5′-TGCACCTTCTTCAGAGCCACT-3′), | (SEQ ID NO:33) |
| with | |

Nek6 specific product size of 89 bp; and

“TATA box”-binding protein (IBP)-F primer (TGATGCCCTTCTGTAAGTGCC) (SEQ ID NO:34) and

TBP-R primer (GCACGGTATGAGCAACTCACA) (SEQ ID NO:35), with a TBP-specific product size of 101 base pairs (bp).

The thermal cycling conditions comprised an initial RT reaction step at 48° C. for 30 min and 40 cycles at 95° C. for 30 sec and 65° C. for min. Accumulation of PCR product was monitored in real time (Mx4000, Stratagene, La Jolla, Calif.), with appropriate controls. A standard curve was generated for each target and the amount of each mRNA relative to total RNA was determined using the Ct method; the amount of Nek6 RNA was divided by the amount of TBP RNA.

Results

Nek6 Activation Requires Phosphorylation of Ser206 in the Activation Loop

Overexpressed wild type FLAG Nek6 (referred to simply as “Nek6” in these studies and corresponding figures) migrates as a doublet on SDS-PAGE (FIG. 7A), whereas the kinase inactive mutant, FLAG Nek6 (K74M/K75M) migrates as a single band corresponding in mobility to the more rapidly migrating band of wild type Nek6 (Belham et al., Curr. Biol., 11: 1155-1167 (2001)). Only the slower moving Nek6 band exhibits auto-phosphorylation in a washed immunoprecipitate and only this upper band catalyzes MBP phosphorylation after renaturation in-an “in-gel” kinase assay (FIG. 7B). Treatment of mammalian recombinant Nek6 with protein phosphatase 2A in vitro abolishes Nek6 kinase activity and eliminates the slower migrating band of Nek6 polypeptide on SDS-PAGE (Belham et al., 2001). These features indicate that the activity of recombinant Nek6 is dependent on Nek6 polypeptide phosphorylation, probably catalyzed by another protein kinase.

Wild type FLAG-tagged Nek6, immunopurified after transient expression in HEK293 cells was subjected to SDS-PAGE, each Coomassie blue-stained band of the closely spaced FLAG Nek6 doublet (FIG. 7A) was excised, digested with trypsin, and the digests were analyzed by LC-MS-MS, so as to identify sites of phosphorylation. The inactive, more rapidly migrating band (“band b”) yielded a single phosphopeptide surrounding Ser206 having amino acid sequence FFSSETTAAHS₂₀₆LVGTPYYMSPER (amino acids 196-218 of SEQ ID NO:4) in the activation loop, whereas the peptides derived from the slower migrating, active Nek6 band (“band a”) yielded one peptide having an amino acid sequence of FFSSETT₂₀₂AAHS₂₀₆LVGTPYYMSPER (amino acids 196-218 of SEQ ID NO:4), which exhibited phosphorylation at Ser206 and Thr202, and another peptide having an amino acid sequence of HPNTLS₃₇FR (amino acids 32-39 of SEQ ID NO:2), which exhibited phosphorylation at Ser37, with the peptide segment encompassing Thr202/Ser206 yielding roughly equal amounts of peptides phosphorylated at Ser206 exclusively and those phosphorylated at both Ser206 and Thr202. Peptides phosphorylated at Thr202 exclusively were not observed. Therefore, polyclonal anti-phosphopeptide antibodies were prepared that were specific for Nek6 phosphorylated at Ser206 or Thr202 as described above. Immunoblot (IB) of transiently expressed FLAG Nek6 indicates that detectable immunoreactivity with either antibody was evident only in the upper, more slowly moving band of Nek6 polypeptide (FIG. 7C). It is likely that the detection by MS-MS of Ser206-P site in the bottom, faster Nek6 band reflects trace contamination by the upper band, a view supported by the finding that mutation of Ser206 to Asp (S206D) results in a slowing of the Nek6 polypeptide on SDS-PAGE (FIG. 8A). Transiently expressed mutant, inactive Nek6 (K74M/K75M) yielded no phosphopeptides on MS-MS analysis of tryptic digests; the tryptic peptide encompassing Ser206/Thr202 was identified only in its unphosphorylated state.

The activity of a variety of Nek6 site-specific mutations was characterized (FIG. 8A). Conversion of Ser206 to Ala (S206A) reduced Nek6 activity by 98%, and Ser206Asp (S206D) mutant, although upshifted in mobility on SDS-PAGE, exhibited only approximately 5 to 10% of wild type activity. Conversion of Thr202 to Ala (T202A) or Cys (T202C), or conversion of the adjacent Thr residues at 201 and 202 both to Ala (T201A/T202A) reduced Nek6 activity by 75-80%, whereas conversion of the nearby pair of Ser residues at 198 and 199 to Ala (S198A/S 199A) had no effect on Nek6 activity. Introduction of a Thr202 to Glu (T202E) mutation into wild type Nek6 increases the apparent specific activity by approximately 20%, however introduction of Thr202 to Glu (T202E) into the Ser206 to Asp (S206D) background increases the low activity of this mutant by approximately threefold, so that the double mutant (T202E, S206D) exhibits approximately 20% of wild type activity. These results indicate that Nek6 activation is absolutely dependent on Ser206 phosphorylation, and support the likelihood that phosphorylation at Thr202, although not indispensable or of primary importance, further augments Nek6 activity.

The primary role of Nek6 Ser206 phosphorylation is further supported by the findings on two dimensional ³²P-labeled tryptic peptides maps of transiently expressed wild type and mutant Nek6 polypeptides (FIG. 8B), which demonstrate that phosphorylation of Ser206 is necessary for the phosphorylation of other Nek6 sites. The ³²P incorporation in vivo into Nek6 (S206A) is reduced by greater than 80% as compared to wild type Nek6 (FIG. 8B-1). Moreover, whereas two dimensional ³²P tryptic peptides maps of ³²P-Nek6 exhibit a single dominant ³²P-peptide, two minor ³²P-peptides, and several trace ³²P-peptides, peptide maps of ³²P Nek6 (S206A) lack all three of the characteristic ³²P-peptides, and exhibit only a faint background of the multiple trace, presumably nonspecific, ³²P-peptides (see, FIG. 8B-2). The overall ³²P incorporation into the Nek6 (T201A/T202A) mutant is also substantially reduced from wild type (FIG. 8B-1) however, the ³²P-tryptic peptide map of this variant contains each of the three predominant ³²P-peptides seen in digests of wild type Nek6, in a proportion similar to that seen in the wild type Nek6 (FIG. 8B-1). This pattern indicates that the mutation of Thr202 affects Nek6 activity primarily by reducing the extent of Ser206 phosphorylation, and secondarily by the loss of its own phosphorylation. The persistence of the three major ³²P-peptides in the Nek6 T201A/T202A mutant suggests the occurrence in this mutant of phosphorylation at other sites situated on the same tryptic peptide (e.g., Thr198 or Thr199).

In summary, the data from the above studies indicate that Nek6 activity requires phosphorylation of Ser206 on the activation loop of the Nek6 protein (corresponding to Ser195 in Nek7). Ser206 phosphorylation also appears to facilitate Thr202 phosphorylation, which may increase catalytic activity somewhat further. As to the mechanism by which these phosphorylations are accomplished in vivo, and while not intending to be bound, the absence of detectable phosphorylation in the Nek6 ATP site mutant (K74M/K75M) suggests that either Ser206 phosphorylation is catalyzed by intramolecular auto-phosphorylation; or alternatively, Nek6 (K74M/K75M) interacts poorly with an upstream kinase. The evidence from the study discussed next strongly supports the latter explanation, at least in mammalian cells.

Nercc1 Binds, Phosphorylates, and Activates Nek6 and Nek7

Recombinant wild type FLAG Nek6 expressed in HEK 293 cells is recovered in association with endogenous Nercc1 (see, Examples 2 and 4, above; Roig et al., Genes Dev., 16 1640-1658 (2002)). FLAG Nek6 immunoprecipitates (IP) prepared from HEK293 cells invariably reveal the presence of endogenous Nercc1, which is retained despite exhaustive washes of the Nek6 immunoprecipitate in RIPA buffer (see, e.g., FIG. 7B). The tight association between recombinant Nek6 and endogenous Nercc1 indicates a likely functional relationship. Therefore, the physical and functional interactions of Nek6 and Nercc1 were examined further. Transient expression of GST Nek6 yields substantial recovery of coexpressed wild type FLAG Nercc1 polypeptide on GSH-agarose affinity pull downs (FIG. 9A). Wild type GST Nek6 also binds avidly to the catalytically inactive Nercc1 (K8μM) and the constitutively active Nercc1 mutant, Nercc (Δ347-732). These results are consistent with the previous demonstration (see, Example 4, above; Roig et al., Genes Dev., 16 1640-1658 (2002)), that the Nek6 binding site maps between Nercc1 amino acids 732 and 891 of SEQ ID NO:2. Catalytically inactive Nek6 (K74M/K75M) is expressed at much lower abundance as compared with wild type Nek6. Nevertheless, when its level of expression is comparable to that of wild type Nek6, it is evident that Nek6 (K74M/K75M) is greatly impaired in its ability to bind Nercc1 (FIG. 9A).

Overexpression of wild type Nek6 with the constitutively activated Nercc1 mutant, Nercc1 (Δ347-732), results in an observable upshift in the electrophoretic mobility of Nek6 (FIG. 9B and FIG. 9A, lane 3 from left) and in a two-fold increase in the specific activity of coexpressed Nek6 (FIG. 9B). Thus, active Nercc1 is able to induce Nek6 phosphorylation and activation in vivo; the modest stimulation of Nek6 activity by Nercc1 (Δ347-732) probably reflects the already high basal activity of recombinant overexpressed Nek6. Nercc1 (Δ347-732) also slows the electrophoretic mobility of the coexpressed inactive Nek6 mutant, Nek6 (K74M/K75M) (FIG. 9C). Tryptic digests of Nek6 (K74M/K75M) coexpressed with Nercc1 (Δ347-732) exhibited on LC/MS/MS considerable phosphorylation at Ser206 and small amounts of peptide phosphorylated at both Ser206 and Thr202. These findings were subsequently confirmed on anti-phosphopeptide immunoblots (FIG. 9C). The ability of coexpressed Nercc1 (Δ347-732) to promote these phosphorylations of Nek6 (K74M/K75M) requires an active Nercc1 catalytic domain. Thus, coexpression of Nek6 with active Nercc1 results in phosphorylation at Ser206 and a modest increase in the already substantial activity of coexpressed recombinant Nek6.

Next, whether Nercc1 could directly phosphorylate Nek6 was examined. Immunopurified, recombinant wild type Nercc1 activated in vitro by auto-phosphorylation catalyzed the phosphorylation of a mammalian recombinant Nek6 (K74M/K75M) substrate at Ser206 (FIG. 10A). This modification occurred, however, with a rather low efficiency, consistent with the very poor binding of Nek6 (K74M/K75M) to Nercc1. Notably, all of the Ser206-phosphorylated Nek6 (K74M/K75M) polypeptide co-precipitated with Nercc1 (FIG. 10B), consistent with the view that the stable binding of Nek6 to Nercc1 is critical for Nercc1 -catalyzed Nek6 phosphorylation.

In light of the above findings, wild type Nek6 was used as substrate for Nercc1 phosphorylation. For this study, inasmuch as Nek6 overexpressed in mammalian cells shows high levels of Ser206 phosphorylation and kinase activity, the recombinant fusion proteins GST Nek6 and GST Nek7 were used. Surprisingly, GST Nek6, which expresses poorly in bacteria and is predominantly insoluble, exhibited varying extents of Ser206 phosphorylation and spontaneous kinase activity depending on the batch (data not shown). In contrast, bacterial recombinant GST Nek7 never showed activation loop (Ser195) phosphorylation or significant kinase activity when purified from bacteria. The “inactive” preparations of bacterial recombinant GST Nek6 and GST Nek7 were examined for their ability to be phosphorylated and activated by Nercc1 (FIG. 10C). A computer search using the putative motif for Nek6 phosphorylation specificity (Lizcano et al., J. Biol. Chem., 277: 27839-27849 (2002)) identified several candidate Nek6 substrates; a GST-fusion of the extreme carboxy terminus of Cdc16 that contains several putative Nek6 phosphorylation sites was readily phosphorylated by Nek6 and Nek7, but not significantly phosphorylated by active Nercc1 kinase. GST Nek6 and GST Nek7 were incubated with Mg²⁺ ATP in the presence or absence of active Nercc1 kinase; after 30 minutes, GST-Cdc16-Ct and tracer [γ-32P]ATP were added for an additional 30 minutes. As shown in FIG. 10C, Nercc1 catalyzed the phosphorylation of Nek6 (Ser206) and the equivalent site on Nek7 (Ser195), resulting in a 20-25 fold activation of Nek6/7 kinase activity. In contrast, Nek7 which is devoid of basal phosphorylation at Ser195 exhibited no ability to catalyze auto-phosphorylation at Ser195 during this in vitro incubation. Nek6 exhibited a low level of basal phosphorylation at Ser206 that did not change during the incubation with Mg²⁺ ATP. A similar experiment using active Nercc1 and GST Nek7 was analyzed using an “in gel” kinase assay with MBP as substrate (FIG. 10D); an upshift in Nek7 mobility was evident after Nercc1-catalyzed Nek7 phosphorylation and the “activated” MBP kinase activity co-migrates entirely with the upshifted band of Nek7.

Inasmuch as the binding of Nek6 to Nercc1 appeared to be critical for the Nercc1 catalyzed phosphorylation of Nek6 in vitro (see, FIG. 10B), the effect of over-expressing the Nercc1 noncatalytic tail on the activity of coexpressed Nek6 was examined. A FLAG-tagged polypeptide consisting of Nercc1 amino acids 347-979 of SEQ ID NO:2 strongly inhibited the activity of coexpressed GST Nek6 (FIG. 11A), concomitant with decreased Nek6 phosphorylation at Ser206 and Thr202 (FIG. 11B), i.e., decreased Nek6 activation. Interestingly, the noncatalytic tail of Nercc1 (amino acids 732-979 of SEQ ID NO:2) was also able to directly inhibit the enzymatic activity of preactivated, wild type Nek6 in vitro (see, FIG. 11C). Nek6 phosphorylated the recombinant GST Nercc1 (732-979) polypeptide, however substantial inhibition of the phosphorylation of the Nek6 substrate (i.e., p70S6K ΔCT104 in this experiment) is evident at GST Nercc1 (A732-979) concentrations far below those of the p70S6K ΔCT104 polypeptide, and the Nek6-catalyzed phosphorylation of GST Nercc1 (732-979) polypeptide actually diminished as the concentration of GST Nercc1 (732-979) polypeptide was increased. These data indicate the occurrence of a noncompetitive mode of inhibition of Nek6 catalytic activity by GST Nercc1 (732-979) polypeptide. Thus, the binding of Nek6 to the Nercc1 noncatalytic tail serves not only to facilitate Nek6 activation by active Nercc1, but possibly to restrict the catalytic activity of Nek6 upon activation by Nercc1, and perhaps direct it to specific targets.

Nek6, Like Nercc1, is Activated in Mitosis

The regulation of endogenous Nek6 activity in vivo was examined. An affinity purified anti-Nek6 peptide antibody raised against a polypeptide comprising an amino terminal murine Nek6 peptide AGQPSHMPHGGSPN (SEQ ID NO:30) conjugated to KLH via a cysteine residue as described above, gave highly specific immunoblots and was capable of modest immunoprecipitation of the endogenous Nek6 polypeptide, although at very low efficiency. Thus far, useful antibodies reactive with endogenous Nek7 have not been obtained. Nek6 immunoreactivity was evident in several commonly used mammalian cell lines including COS7 cells (FIG. 12A). The regulation of Nek6 in the rat hepatoma line H4IIEC was examined because Nek6 mRNA shows highest abundance in liver (Belham et al., 2001). Moreover, Nek6 had been isolated as a candidate p70S6 Kinase-Thr412 kinase, and insulin regulation of endogenous p70S6 Kinase (also referred to as p70S6K) had previously been characterized in this cell line (see, Price et al., Proc. Natl. Acad. Sci. USA, 87: 7944-7948 (1990)). Insulin generated a rapid, fifteen-fold activation of endogenous p70S6Kinase in serum-deprived H4 cells; under these conditions, however, no change occurred in the basal activity of immunoprecipitated endogenous Nek6 (FIG. 12B). The activity of p70S6K in insulin-stimulated H4 cells was inhibited progressively by increasing concentrations of wortmannin (IC50 of approximately 30 nM, FIG. 12C) or rapamycin (IC50 of approximately 2 nM, FIG. 12D); these inhibitors of PI-3 kinase and mTOR, respectively, did not alter significantly the activity of Nek6. Taken together, these results indicate that Nek6 activity is not rapidly regulated by signal transduction pathways downstream of the insulin receptor and it is therefore unlikely that Nek6 functions as an activating p70S6K-kinase in response to activation of receptor tyrosine kinases. The specific activity of Nek6 in H4 cells is, however, sensitive to serum withdrawal and declines progressively by 80% over 48 hours (FIG. 12E). This decline in Nek6 activity, however, is much slower and less severe than that observed for the p70S6K.

The fall in Nek6 activity as H4 cells are brought to quiescence by serum withdrawal together with the previous observation that Nercc1 activity increases at mitosis (see, Example 11, above; Roig et al., Genes Dev., 16: 1640-1658 (2002)), led to the examination of the abundance and activity of Nek6 in cells arrested in mitosis by nocodazole, as compared to non-mitotic cells. It is evident (FIG. 13A) that the relative abundance of Nek6 was increased by 3-4 fold in H4 cells arrested in mitosis as compared with the amount in the non-mitotic cells; moreover, Nek6 mobility on SDS-PAGE was retarded in mitotic cells and Nek6 kinase activity increased in parallel to its abundance. These findings indicate that endogenous Nek6, like Nercc1, is activated in mitosis. Mitotic HeLa cells also showed an increase in the abundance of the Nek6 polypeptide, and a marked increase in Nek6 phosphorylation at Ser206 and Thr202 (FIG. 13B); however, direct assay of Nek6 activity in this cell line was precluded as it was not possible to immunoprecipitate endogenous HeLa Nek6. RTPCR showed that the abundance of Nek6 mRNA (relative to that of TATA-box binding protein) was approximately 3-fold higher in mitotic than in exponentially growing HeLa cells (FIG. 13C), pointing to at least one mechanism for the increase in Nek6 protein content in mitotic cells. The behavior of Nek6 in U2OS cells was similar to that observed in H4 and Hela cells (data not shown). Thus, endogenous Nek6, like Nercc1, is increased in abundance and activated in mitosis.

### Example 13

### Nek6 Levels and Phosphorylation State Change During the Cell Cycle

A time course experiment was carried out to study the synthesis and activation of Nek6 and Nercc1 in mitosis. The abundance and electrophoretic mobility of Nek6 was determined in human U2OS cells in different phases of the cell cycle by western blot using specific antibodies, in parallel to that of Nercc 1. Cyclin B1 was determined by western blot as a mitotic marker, while tubulin was used as a loading control. Results are shown in FIG. 14. The left panel of FIG. 14 shows levels and electrophoretic mobility of Nek6 and Nercc1 as exponentially growing cells entered into mitosis after G1/S arrest (G1/S, at t=0). Cells were arrested in G1/S by 2 μg/ml aphidicolin for 6-24 hours, thereafter cells were released from the aphidicolin block into media containing 500 ng/ml nocodazol. The right panel of FIG. 14 shows accumulation and activation of Nek6 and Nercc1 as cells exited from mitosis at 30 minutes, 60 minutes, and 4 hours after the nocodazol arrest. The results indicate that Nek6 protein amounts vary with the cell cycle, being maximal during mitosis, and decreasing sharply during mitotic exit; the observed accumulation of Nek6 is accompanied by an upshift of the protein kinase and parallels Nercc1 activation.

All patents, applications, and publications cited in the above text are incorporated herein by reference.

Other variations and embodiments of the invention described herein will now be apparent to those of ordinary skill in the art without departing from the scope of the invention or the spirit of the claims below.

What is claimed is:

1. A method of identifying a compound that is an inhibitor of mitosis comprising the steps of:
(a) providing a kinase reaction mixture comprising a purine nucleoside triphosphate, a Nercc1 kinase protein, and a kinase substrate,
(b) incubating said kinase reaction mixture in the presence and absence of a test compound for a time sufficient to permit the Nercc1 kinase protein to phosphorylate the kinase substrate, and
(c) determining the level of phosphorylated kinase substrate in the presence and absence of said test compound,
wherein a lower level of phosphorylated kinase substrate produced in the presence of said test compound compared to the level produced in the absence of said test compound indicates that said test compound is an inhibitor of mitosis.

2. The method according to claim 1, wherein the Nercc1 kinase protein is initially a non-activated Nercc1 kinase protein capable of auto-activation by auto-phosphorylation.

3. The method according to claim 1, wherein the Nercc1 kinase protein and the kinase substrate are an initially non-activated Nercc1 kinase protein or fusion protein thereof, wherein said initially non-activated Nercc1 kinase or fusion protein is capable of auto-activation by auto-phosphorylation.

4. The method according to claim 1, wherein the Nercc1 kinase protein of the method is an activated Nercc1 kinase.

5. The method according to claim 4, wherein the activated Nercc1 kinase is selected from the group consisting of a phosphorylated Nercc1 kinase; a recombinantly produced, activated Nercc1 kinase; a constitutively active mutant variant of Nercc1; and a fusion protein thereof.

6. The method according to claim 5, wherein said activated Nercc1 kinase is a constitutively active mutant variant of Nercc1 or a fusion protein thereof.

7. The method according to claim 6, wherein said constitutively active mutant variant of Nercc1 or fusion protein thereof is constitutively active owing to the absence of all or a portion of an RCC1 auto-inhibitory domain.

8. The method according to claim 1 or claim 2, wherein the kinase substrate is selected from the group consisting of a non-activated Nercc1 kinase protein, a non-activated Nek6 protein, a non-activated Nek7 protein, a histone, a casein, a myelin basic protein (MBP), and a fusion protein thereof.

9. The method according to claim 8, wherein the kinase substrate is a histone or a fusion protein comprising a histone.

10. The method according to claim 9, wherein said histone is histone H3 or histone H4.

11. The method according to claim 1 or claim 2, wherein said purine nucleoside triphosphate comprises an adenosine triphosphate or a guanosine triphosphate.

12. The method according to claim 1 or claim 2, wherein said purine nucleoside triphosphate is radio-labeled with ³²P.

13. The method according to claim 1 or claim 2, wherein step (c) of the method for determining the level of phosphorylated kinase substrate is carried out using an antibody that specifically binds the phosphorylated form of the kinase substrate.

14. The method according to claim 13, wherein said antibody specifically binds a phosphorylated kinase substrate selected from the group consisting of a phosphorylated Nercc1 kinase protein, a phosphorylated Nek6 protein, a phosphorylated Nek7 protein, a phosphorylated histone, a phosphorylated casein, a phosphorylated MBP, and a fusion protein thereof.

15. The method according to claim 1 or 2, wherein at least one step of said method is carried out in a vessel comprising a test tube, a microtiter plate, a biochip, a coverslip, and combinations thereof.

16. The method according to claim 1 or 2, wherein at least one step is carried out automatically or semi-automatically.

17. The method according to claim 1 or claim 2, comprising the additional step:
(d) determining whether said test compound of step (c) inhibits mitosis in dividing cells.

18. The method according to claim 17, wherein said step (d) comprises contacting dividing cells in a culture with said test compound and assaying the cells a response selected from the group consisting of cell lysis, apoptosis, disruption of mitotic spindles, misalignment of chromosomes, and combinations thereof.

19. The method according to claim 17, wherein said dividing cells are cancer cells.

20. The method according to claim 17, wherein said step (d) is carried out in a vessel selected from the group consisting of a test tube, a microtiter plate, a biochip, a coverslip, and combinations thereof.

21. The method according to claim 17, wherein said step (d) is carried out automatically or semi-automatically.

22. A method of identifying an inhibitor of mitosis comprising:
(a) providing a kinase reaction mixture comprising an activated Nek6 or Nek7 linase protein, a kinase substrate, and a purine nucleoside triphosphate,
(b) incubating said reaction mixture in the presence and absence of a test compound for a time sufficient to permit the activated Nek6 or Nek7 kinase protein to phosphorylate said kinase substrate, and
(c) detecting the level of phosphorylated kinase substrate in the presence and absence of said test compound,
wherein a lower level of phosphorylated kinase substrate produced in the presence of said test compound compared to the level produced in the absence of said test compound indicates that said test compound is an inhibitor of mitosis.

23. The method according to claim 22, wherein said kinase substrate is Cdc16, MBP, or a fusion protein thereof.

24. The method according to claim 22, wherein said purine nucleoside triphosphate is an adenosine triphosphate or a guanosine triphosphate.

25. The method according to claim 22, wherein said purine nucleoside triphosphate is radio-labeled with ³²P

26. The method according to claim 22, wherein the step of determining the level of phosphorylated substrate kinase in step (c) of the method is carried out using an antibody that specifically binds the phosphorylated substrate kinase.

27. The method according to claim 26, wherein said antibody specifically binds a phosphorylated substrate kinase selected from the group consisting of a phosphorylated Cdc16, a phosphorylated MBP, and a fusion protein thereof.

28. The method according to claim 22, wherein at least one step is carried out in a vessel selected from the group consisting of a test tube, a microtiter plate, a biochip, a coverslip, and combinations thereof.

29. The method according to claim 22, wherein at least one step is carried out automatically or semi-automatically.

30. The method according to claim 22, comprising the additional step:
(d) determining whether said test compound inhibits mitosis in dividing cells.

31. The method according to claim 30, wherein step (d) comprises contacting dividing cells in a culture with said test compound and assaying the cells for a response selected from the group consisting of cell lysis, apoptosis, disruption of mitotic spindles, misalignment of chromosomes, and combinations thereof.

32. The method according to claim 31, wherein said dividing cells are cancer cells.

33. The method according to claim 30, wherein step (d) is carried out in a vessel comprising a test tube, a microtiter plate, a biochip, a coverslip, and combinations thereof.

34. The method according to claim 30, wherein step (d) is carried out automatically or semi-automatically.

35. A method of diagnosing a cancerous or potentially cancerous state in an individual comprising:
(a) assaying cells from said individual for the level of Nercc1, Nek6, or Nek7 kinase protein or corresponding kinase activity,
(b) comparing the level of Nercc1, Nek6, or Nek7 protein or corresponding kinase activity determined in the cells from step (a) to the level determined in normal, non-cancerous cells or to the level determined in a prior sample of cells from said individual,
wherein an elevation in the level of Nercc1, Nek6, or Nek7 protein or corresponding kinase activity determined in the cells from step (a) relative to the level determined in normal, non-cancerous cells or relative to the level determined in a prior sample of cells from said individual is diagnostic for a cancerous or potentially cancerous state.

36. The method according to claim 35, wherein the level of Nercc1, Nek6, or Nek7 in step (a) is detected by a method selected from the group consisting of a kinase phosphate transfer reaction, an immunodection assay, and a transcription assay for mRNA encoding Nercc1, Nek6, or Nek7.

37. The method according to claim 35, wherein the cells of an individual in step (a) are obtained from a source selected from the group consisting of a tissue biopsy, blood, cell smear, tissue swab, bodily fluid, and feces.

38. A mutant variant Nercc1 kinase protein or fusion protein thereof that is constitutively active, wherein said mutant variant Nercc1 kinase protein or fusion protein thereof is active in the absence of phosphorylation at an activation site in said mutant variant Nercc1 kinase protein or fusion protein thereof.

39. The mutant variant Nercc1 kinase protein or fusion protein thereof according to claim 38, wherein said mutant variant Nercc1 kinase protein or fusion protein thereof lacks all or a portion of an RCC1 auto-inhibitory domain.

40. A mutant variant Nercc1 protein lacking all or a sufficient portion of an RCC1 auto-inhibitory domain so that said mutant variant Nercc1 protein is permanently activated to provide a functional Nercc1 kinase activity without the necessity of undergoing phosphorylation.

41. The mutant variant Nercc1 protein according to claim 41, wherein said mutant variant Nercc1 protein is Nercc1 (Δ347-732).

42. A mutant variant of Nercc1 selected from the group consisting of Nercc1 (Δ347-732), Nercc (Δ763-889), Nercc1 (347-732), Nercc (338-739), and a fusion protein thereof.

43. A fusion protein comprising a non-Nercc polypeptide linked in frame with a mutant variant of Nercc1 selected from the group consisting of Nercc1 (Δ347-732), Nercc (Δ763-889), Nercc1 (347-732), and Nercc (338-739).

44. The fusion protein according to any one of claims 38, 39, 42, and 43, wherein said fusion protein comprises a non-Nercc polypeptide selected from the group consisting of glutathionine S-transferase (GST), an epitope tag polypeptide, and combinations thereof.

45. The fusion protein according to claim 44, wherein said epitope tag polypeptide is selected from the group consisting of FLAG, HA, myc, and combinations thereof


SEQUENCE LISTING (35 sequences)

SEQ ID NO: 1 - DNA, 2937 bp, Homo sapiens
atgtcggtgctgggcgagtacgagcgacactgcgattccatcaactcggactttgggagcgagtccgggggttgcggggactcgagtccggggcctagcgccagtcaggggccgcgagccggcggcggcgcggcggagcaggaggaactgcactacatccccatccgcgtcctgggccgcggcgccttcggggaagccacgctgtaccgccgcaccgaggatgactcactggttgtgtggaaggaagtcgatttgacccggctgtctgagaaggaacgtcgtgatgccttgaatgagatagttattctggcactgctgcagcacgacaacattattgcctactacaatcacttcatggacaataccacgctgctgattgagctggaatattgtaatggagggaacctgtatgacaaaatccttcgtcagaaggacaagttgtttgaggaagagatggtggtgtggtacctatttcagattgtttcagcagtgagctgcatccataaagctggaatccttcatagagatataaagacattaaatatttttctgaccaaggcaaacctgataaaacttggagattatggcctagcaaagaaacttaattctgagtattccatggctgagacgcttgtgggaaccccatattacatgtctccagagctctgtcaaggagtaaagtacaatttcaagtctgatatctgggcagttggctgcgtcatttttgaactgcttaccttaaagaggacgtttgatgctacaaacccacttaacctgtgtgtgaagatcgtgcaaggaattcgggccatggaagttgactctagccagtactctttggaattgatccaaatggttcattcgtgccttgaccaggatcctgagcagagacctactgcagatgaacttctagatcgccctcttctcaggaaacgcaggagagagatggaggaaaaagtcactctgcttaatgcacctacaaagagaccaaggtcaagcactgtgactgaagcacccattgctgtagtaacatcacgaaccagtgaagtctatatttggggtggtggaaaatccaccccccagaaactggatgttatcaagagtggctgtagtgcccggcaggtctgtgcagggaatacccactttgctgtggtcacagtggagaaggaactgtacacttgggtgaacatgcaaggaggcactaaactccatggtcagctgggccatggagacaaagcctcctatcgacagccaaagcatgtggaaaagttgcaaggcaaagctatccatcaggtgtcatgtggtgatgatttcactgtctgtgtgactgatgagggtcagctctatgccttcggatcagattattatggctgcatgggggtggacaaagttgctggccctgaagtgctagaacccatgcagctgaacttcttcctcagcaatccagtggagcaggtctcctgtggagataatcatgtggtggttctgacacgaaacaaggaagtctattcttggggctgtggcgaatatggacgactgggtttggattcagaagaggattattatacaccacaaaaggtggatgttcccaaggccttgattattgttgcagttcaatgtggctgtgatgggacatttctgttgacccagtcaggcaaagtgctggcctgtggactcaatgaattcaataagctgggtctgaatcagtgcatgtcgggaattatcaaccatgaagcataccatgaagttccctacacaacgtcctttaccttggccaaacagttgtccttttataagatccgtaccattgccccaggcaagactcacacagctgctattgatgagcgaggccggctgctgacctttggctgcaacaagtgtgggcagctgggcgttgggaactacaagaagcgtctgggaatcaacctgttggggggaccccttggtgggaagcaagtgatcagggtctcctgcggtgatgagtttaccattgctgccactgatgataatcacatttttgcctggggcaatggtggtaatggccgcctggcaatgacccccacagagagaccacatggctctgatatctgtacctcatggcctcggcctatttttggatctctgcatcatgtcccggacctgtcttgccgtggatggcataccattctcatcgttgagaaagtattgaattctaagaccatccgttccaatagcagtggcttatccattggaactgtgtttcagagctctagcccgggaggaggcggcgggggcggcggtggtgaagaagaggacagtcagcaggaatctgaaactcctgacccaagtggaggcttccgaggaacaatggaagcagaccgaggaatggaaggtttaatcagtcccacagaggccatggggaacagtaatggggccagcagctcctgtcctggctggcttcgaaaggagctggaaaatgcagaatttatccccatgcctgacagcccatctcctctcagtgcagcgttttcagaatctgagaaagataccctgccctatgaagagctgcaaggactcaaagtggcctctgaagctcctttggaacacaaaccccaagtagaagcctcgtcacctcggctgaatcctgcagtaacctgtgctgggaagggaacaccactgactcctcctgcgtgtgcgtgcagctctctgcaggtggaggttgagagattgcagggtctggtgttaaagtgtctggctgaacaacagaagctacagcaagaaaacctccagatttttacccaactgcagaagttgaacaagaaattagaaggagggcagcaggtggggatgcattccaaaggaactcagacagcaaaggaagagatggaaatggatccaaagcctgacttagattcagattccgggtgcctcctgggaacagactcctgtagacccagcctc

SEQ ID NO: 2 - protein, 979 aa, Homo sapiens
MSVLGEYERHCDSINSDFGSESGGCGDSSPGPSASQGPRAGGGAAEQEELHYIPIRVLGRGAFGEATLYRRTEDDSLVVWKEVDLTRLSEKERRDALNEIVILALLQHDNIIAYYNHFMDNTTLLIELEYCNGGNLYDKILRQKDKLFEEEMVVWYLFQIVSAVSCIHKAGILHRDIKTLNIFLTKANLIKLGDYGLAKKLNSEYSMAETLVGTPYYMSPELCQGVKYNFKSDIWAVGCVIFELLTLKRTFDATNPLNLCVKIVQGIRAMEVDSSQYSLELIQMVHSCLDQDPEQRPTADELLDRPLLRKRRREMEEKVTLLNAPTKRPRSSTVTEAPIAVVTSRTSEVYIWGGGKSTPQKLDVIKSGCSARQVCAGNTHFAVVTVEKELYTWVNMQGGTKLHGQLGHGDKASYRQPKHVEKLQGKAIHQVSCGDDFTVCVTDEGQLYAFGSDYYGCMGVDKVAGPEVLEPMQLNFFLSNPVEQVSCGDNHVVVLTRNKEVYSWGCGEYGRLGLDSEEDYYTPQKVDVPKALIIVAVQCGCDGTFLLTQSGKVLACGLNEFNKLGLNQCMSGIINHEAYHEVPYTTSFTLAKQLSFYKIRTIAPGKTHTAAIDERGRLLTFGCNKCGQLGVGNYKKRLGINLLGGPLGGKQVIRVSCGDEFTIAATDDNHIFAWGNGGNGRLAMTPTERPHGSDICTSWPRPIFGSLHHVPDLSCRGWHTILIVEKVLNSKTIRSNSSGLSIGTVFQSSSPGGGGGGGGGEEEDSQQESETPDPSGGFRGTMEADRGMEGLISPTEAMGNSNGASSSCPGWLRKELENAEFIPMPDSPSPLSAAFSESEKDTLPYEELQGLKVASEAPLEHKPQVEASSPRLNPAVTCAGKGTPLTPPACACSSLQVEVERLQGLVLKCLAEQQKLQQENLQIFTQLQKLNKKLEGGQQVGMHSKGTQTAKEEMEMDPKPDLDSDSGCLLGTDSCRPSL

SEQ ID NO: 3 - DNA, 939 bp, Homo sapiens
atggcaggacagcccggccacatgccccatggagggagttccaacaacctctgccacaccctggggcctgtgcatcctcctgacccacagaggcatcccaacacgctgtcttttcgctgctcgctggcggacttccagatcgaaaagaagataggccgaggacagttcagcgaggtgtacaaggccacctgcctgctggacaggaagacagtggctctgaagaaggtgcagatctttgagatgatggacgccaaggcgaggcaggactgtgtcaaggagatcggcctcttgaagcaactgaaccacccaaatatcatcaagtatttggactcgtttatcgaagacaacgagctgaacattgtgctggagttggctgacgcaggggacctctcgcagatgatcaagtactttaagaagcagaagcggctcatcccggagaggacagtatggaagtactttgtgcagctgtgcagcgccgtggagcacatgcattcacgccgggtgatgcaccgagacatcaagcctgccaacgtgttcatcacagccacgggcgtcgtgaagctcggtgaccttggtctgggccgcttcttcagctctgagaccaccgcagcccactccctagtggggacgccctactacatgtcaccggagaggatccatgagaacggctacaacttcaagtccgacatctggtccttgggctgtctgctgtacgagatggcagccctccagagccccttctatggagataagatgaatctcttctccctgtgccagaagatcgagcagtgtgactaccccccactccccggggagcactactccgagaagttacgagaactggtcagcatgtgcatctgccctgacccccaccagagacctgacatcggatacgtgcaccaggtggccaagcagatgcacatctggatgtccagcacc

SEQ ID NO: 4 - protein, 313 aa, Homo sapiens
MAGQPGHMPHGGSSNNLCHTLGPVHPPDPQRHPNTLSFRCSLADFQIEKKIGRGQFSEVYKATCLLDRKTVALKKVQIFEMMDAKARQDCVKEIGLLKQLNHPNIIKYLDSFIEDNELNIVLELADAGDLSQMIKYFKKQKRLIPERTVWKYFVQLCSAVEHMHSRRVMHRDIKPANVFITATGVVKLGDLGLGRFFSSETTAAHSLVGTPYYMSPERIHENGYNFKSDIWSLGCLLYEMAALQSPFYGDKMNLFSLCQKIEQCDYPPLPGEHYSEKLRELVSMCICPDPHQRPDIGYVHQVAKQMHIWMSST

SEQ ID NO: 5 - DNA, 906 bp, Homo sapiens
atggatgagcaatcacaaggaatgcaagggccacctgttcctcagttccaaccacagaaggccttacgaccggatatgggctataatacattagccaactttcgaatagaaaagaaaattggtcgcggacaatttagtgaagtttatagagcagcctgtctcttggatggagtaccagtagctttaaaaaaagtgcagatatttgatttaatggatgccaaagcacgtgctgattgcatcaaagaaatagatcttcttaagcaactcaaccatccaaatgtaataaaatattatgcatcattcattgaagataatgaactaaacatagttttggaactagcagatgctggcgacctatccagaatgatcaagcattttaagaagcaaaagaggctaattcctgaaagaactgtttggaagtattttgttcagctttgcagtgcattggaacacatgcattctcgaagagtcatgcatagagatataaaaccagctaatgtgttcattacagccactggggtggtaaaacttggagatcttgggcttggccggtttttcagctcaaaaaccacagctgcacattctttagttggtacgccttattacatgtctccagagagaatacatgaaaatggatacaacttcaaatctgacatctggtctcttggctgtctactatatgagatggctgcattacaaagtcctttctatggtgacaaaatgaatttatactcactgtgtaagaagatagaacagtgtgactacccacctcttccttcagatcactattcagaagaactccgacagttagttaatatgtgcatcaacccagatccagagaagcgaccagacgtcacctatgtttatgacgtagcaaagaggatgcatgcatgcactgcaagcagc

SEQ ID NO: 6 - protein, 302 aa, Homo sapiens
MDEQSQGMQGPPVPQFQPQKALRPDMGYNTLANFRIEKKIGRGQFSEVYRAACLLDGVPVALKKVQIFDLMDAKARADCIKEIDLLKQLNHPNVIKYYASFIEDNELNIVLELADAGDLSRMIKHFKKQKRLIPERTVWKYFVQLCSALEHMHSRRVMHRDIKPANVFITATGVVKLGDLGLGRFFSSKTTAAHSLVGTPYYMSPERIHENGYNFKSDIWSLGCLLYEMAALQSPFYGDKMNLYSLCKKIEQCDYPPLPSDHYSEELRQLVNMCINPDPEKRPDVTYVYDVAKRMHACTASS

SEQ ID NO: 7 - DNA, 30 bp, artificial
  sequencing primer for Nercc cDNA
gaagatctacgccgccatgtcggtgctggg

SEQ ID NO: 8 - DNA, 32 bp, artificial
  sequencing primer for Nercc cDNA
gaagatctctagagaagagggcgatctagaag

SEQ ID NO: 9 - DNA, 30 bp, artificial; identical to SEQ ID NO: 7
  sequencing primer for Nercc cDNA

SEQ ID NO: 10 - DNA, 20 bp, artificial
  sequencing primer for Nercc cDNA
gtacagttccttctccactg

SEQ ID NO: 11 - DNA, 20 bp, artificial
  amplification primer for Nercc cDNA
cagtggagaaggaactgtac

SEQ ID NO: 12 - DNA, 20 bp, artificial
  amplification primer for Nercc cDNA
ctgtgtctcctcttcaaagg

SEQ ID NO: 13 - DNA, 31 bp, artificial
  amplification primer for Nercc cDNA
cggctctagacattgctgtagtaacatcacg

SEQ ID NO: 14 - DNA, 37 bp, artificial
  amplification primer for Nercc cDNA
cggctctagacttagccactgctattggaacggatgg

SEQ ID NO: 15 - DNA, 31 bp, artificial; identical to SEQ ID NO: 13
  amplification primer for Nercc cDNA

SEQ ID NO: 16 - DNA, 29 bp, artificial
  amplification primer for Nercc cDNA
cggctctagaggggctctataggctcagg

SEQ ID NO: 17 - DNA, 38 bp, artificial
  amplification primer for Nercc cDNA
aaatatgcggccgcaaccatccgttccaatagcagtgg

SEQ ID NO: 18 - DNA, 28 bp, artificial
  amplification primer for Nercc cDNA
cctgatcagggctctataggctcaggag

SEQ ID NO: 19 - DNA, 684 bp, Schistosoma japonicum
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccgggc

SEQ ID NO: 20 - protein, 228 aa, Schistosoma japonicum
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPG

SEQ ID NO: 21 - DNA, 27 bp, artificial
  FLAG epitope tag nucleotide coding sequence
atggactacaaggacgacgatgacaag

SEQ ID NO: 22 - protein, 9 aa, artificial
  FLAG epitope tag amino acid sequence
MDYKDDDDK

SEQ ID NO: 23 - DNA, 30 bp, Influenza virus
atgtacccatacgatgttccagattacgcg

SEQ ID NO: 24 - protein, 10 aa, Influenza virus
MYPYDVPDYA

SEQ ID NO: 25 - DNA, 30 bp, Homo sapiens
gaacaaaaactcatctcagaagaggatctg

SEQ ID NO: 26 - protein, 10 aa, Homo sapiens
EQKLISEEDL

SEQ ID NO: 27 - protein, 13 aa, artificial
  polypeptide for raising antibodies to Nek7
CAAHSLVGTPYYM

SEQ ID NO: 28 - DNA, 9 bp, Homo sapiens
ccgccatgt

SEQ ID NO: 29 - DNA, 6 bp, Homo sapiens
aataaa

SEQ ID NO: 30 - protein, 14 aa, Mus musculus
AGQPSHMPHGGSPN

SEQ ID NO: 31 - protein, 13 aa, artificial
  polypeptide for raising antibodies to Nek6
CGRFFSSETTAAH

SEQ ID NO: 32 - DNA, 21 bp, artificial
  PCR forward primer for analysis of Nek6 RNA
cgaaaagaagataggccgagg

SEQ ID NO: 33 - DNA, 21 bp, artificial
  PCR reverse primer for analysis of Nek6 RNA
tgcaccttcttcagagccact

SEQ ID NO: 34 - DNA, 21 bp, artificial
  PCR forward primer for analysis of TATA box binding protein RNA
tgatgcccttctgtaagtgcc

SEQ ID NO: 35 - DNA, 21 bp, artificial
  PCR reverse primer for analysis of TATA box binding protein RNA
gcacggtatgagcaactcaca